(19) United States
(12) Patent Application Publication
(10) Pub. No.: US 2024/0041987 A1
(43) Pub. Date: Feb. 8, 2024

(54) NOVEL BIOACTIVE PEPTIDE COMBINATIONS AND USES THEREOF

(71) Applicant: COLZYX AB, Lund (SE)

(72) Inventors: Matthias MÖRGELIN, Anderslöv (SE); Suado Mohamed ABDILLAHI, Armdale (AU)

(21) Appl. No.: 18/031,727

(22) PCT Filed: Oct. 15, 2021
(86) PCT No.: PCT/EP2021/078669

§ 371 Date: Apr. 13, 2023

(30) Foreign Application Priority Data
Oct. 16, 2020 (GB) 2016456.2

(51) Int. Cl. A61K 38/39; A61P 17/02; A61P 31/00

(52) U.S. Cl. CPC A61K 38/39; A61P 17/02; A61P 31/00

(57) ABSTRACT

The present invention provides a composition comprising a combination of polypeptides derived from collagen type VI. Also provided are pharmaceutical compositions and kits comprising the composition of the invention. Related aspects provide medical devices, implants, wound care products and materials for use in the same associated with the composition of the invention, and methods of their preparation. Also provided are methods and uses of the composition in the treatment and/or prevention of microbial infections and in wound care, and a method of killing microorganisms in vitro.

### FIELD OF INVENTION

The present invention relates to bioactive peptide combinations comprising collagen VI polypeptides and derivatives thereof in combination with medical devices, implants, wound care products, and kits comprising the same; and uses and medical uses thereof.

### BACKGROUND

Skin is our major external defence system, in charge of protecting our inner body structures from invading microorganisms, and the adverse effects of the external environment. Adult skin is composed of three layers: epidermis or stratum corneum, mainly consisting of keratinocytes; dermis, the connective tissue rich in collagen and elastin; and hypodermis or subcutaneous layer, composed of fat tissue, which provides thermal isolation and mechanical protection to the body [1]. Wounds are superficial or deep injuries within the skin, which may form due to physicochemical or thermal damage. Acute wounds are defined by injured tissues that need a healing period over 8-12 weeks, (e.g., burns, chemical injuries, cuts). In contrast, chronic wounds are a fallout of diseases, such as venous or arterial vascular insufficiency, pressure necrosis, cancer, and diabetes [2,3]. They require longer healing time (weeks-months to years) and often fail to reach a normal healthy state, persisting in a pathological condition of inflammation [4]. Therefore, delayed or impaired wound healing poses a significant socio-economic burden on patients and health care systems worldwide, in terms of treatment costs and waste production [5].

Tissue regeneration after injury is an intricate process where devitalized cellular and tissue structures are replaced [6]. Insight into the carefully orchestrated biochemical and cellular events activated during skin repair is crucial to design appropriate wound dressings [1,7,8]. It comprises extensive changes in cell response as well as in extracellular matrix (ECM) composition. In general, wound repair is divided into different predictable and overlapping phases: haemostasis, inflammation, proliferation, followed by maturation and remodulation of the scar tissue [9-11]. Haemostasis is the immediate response of the body to an injury, in order to stop blood loss at the wound site, by means of fibrin cloths as temporary barriers [12]. Inflammation (from 24 h to 4-6 days) is mediated by neutrophils and macrophages [13], that sweep the wound bed from foreign particles and tissue debris. Cytokines and enzymes are released to stimulate fibroblasts and myofibroblasts macrophages [14], while the wound exudate provides the essential moisture for the recovery. In the proliferation phase epithelialization occurs and newly formed granulation tissue begins to fill the wound area, producing new ECM. Finally, during the remodeling phase, collagen-based cross-linking is responsible for a tight 3D network formation, increasing the tensile strength of the new tissue [12]. Importantly, the intimate relationship between cells and their surrounding framework is commonly regarded to play a pivotal role in regulating regenerative processes. Thus, it is pivotal to create appropriate biomaterials which are extensively loaded with biomolecules in order to ensure premium wound healing properties.

Following the first two phases of wound healing is a repairing phase, termed the proliferative phase. It begins 3 days after the injury and lasts for 2 weeks. After injury, fibroblasts and myofibroblasts proliferate in local wound milieu and are stimulated by TGFβ and PDGF, to migrate to the site of injury on the third day, where they proliferate profusely. Fibroblasts lay extracellular matrix of matrix proteins: hyaluronan, fibronectin, proteoglycans, and extended fibrillar networks consisting of type I/III/V collagen. This matrix helps support cell migration and repair process. Wound contraction, an important reparative process to approximate wound edges, takes place after which fibroblasts are eliminated [15].

Collagen is the most abundant mammalian protein, which provides mechanical strength to tissues and stimulates cell-adhesion and proliferation [16,17]. About thirty different types of collagen have been identified, displaying a triple-helical tertiary structure of polypeptide sequences, but only a few are used in the production of collagen-based biomaterials.

Collagen type VI forms complex and extensive beaded microfibrillar network in most connective tissues. The predominant form of collagen type VI is composed of three distinct polypeptide chains, α1(VI), α2(VI) and α3(VI), which form triple helical monomers (i.e. the monomers do not exist as separate products; the natural product is instead formed of these triple helical monomers). Inside the cell, the monomers assemble into dimers and tetramers that are secreted into the extracellular space. There, the tetramers aggregate end-on-end to form microfibrils that become part of extended supramolecular matrix assemblies. More recently, three additional chains (α4, α5 and α6) were discovered, which may substitute for the α3-chain in some tissues [30, 31]. In terms of structure, each α-chain is characterized by a short extended triple-helical region flanked by two large N- and C-terminal globular regions, which share homology with von Willebrand Factor type A domains (VWA) [32-34]. VWA is also responsible for protein-protein interaction in the ECM [32-35]. The α1(VI) and α2(VI) chains of collagen type VI contain one N-terminal (N1) and two C-terminal (C1 and C2) VWA domains, whereas the α3(VI) is much larger and comprises some ten N-terminal (N10-N1) VWA domains and two C-terminal VWA domains. Additionally, the α3(VI) chain has three C-terminal domains (C3-05) that share homology with salivary gland proteins, fibronectin type III repeats and the Kunitz family of serine protease inhibitors [36]. With its unique setup, collagen type VI provides strength, integrity and structure to wide range of tissues. It is also involved in other important biological processes such as apoptosis, autophagy, angiogenesis, fibrosis and tissue repair [37].

The inventors have previously shown that collagen VI peptides and derivatives thereof actively promote wound healing and have antimicrobial properties (see WO 2017/125585, the content of which is hereby incorporated by reference), making them useful for applications relating to the treatment of hard-to-heal wounds and, at the same time, the killing or growth inhibition of bacteria that have developed resistance to conventional antibiotics, such as MRSA. They are also useful in the stimulation of rapid wound closure and the treatment of microbial infections in contaminated wounds, for example when coated to the surface of a medical device, or incorporated in a medical device, or other such material.

High biocompatibility and biodegradability by endogenous collagenases make collagen ideal for biomedical applications [18,19]. During wound healing, fibroblasts produce collagen molecules that aggregate to form fibrils with diameter in the range of 10-500 nm. This fibrous network facilitates cell migration to the wounded site, actively supporting tissue repair [20]. Thanks to a facile chemical functionalization of the protein structure, various dressing architectures have been exploited. Collagen-based wound dressings, either in forms of hydrogels, electrospun fibers, or nanocrystal-containing scaffolds, have been applied to cover burn wounds, treat ulcers [21-24], reduce tissue contraction and scarring, and increase epithelialization rate [25].

Collagen sponges and fibrous membranes were found particularly promising, due to their wet strength that allows suturing to soft tissues and provides a template for new tissue growth. The multifunctional platform showed anti-bacterial and anti-inflammatory properties, while retaining a favourable topography for cell proliferation, thus accelerating healing and wound closure. Despite their rather extensive usage as biomaterials for scaffold design, collagen scaffolds remain sustainable materials with high engineering potential that is as yet unexplored [26,27].

Given the multiple cellular mechanisms involved in the skin wound healing process and the interplay of several external factors, the choice of suitable dressing materials is compelling. Specifically, for biodegradable natural materials, their degradation needs to follow the dynamics of the wound repair, guaranteeing the physiological healing evolution, and releasing active principles when needed [6]. To address this issue several wound dressings are designed to achieve the highest level of biomimicry by recapitulating the native extracellular matrix biological and physicochemical features. In particular, biological dressings based on native collagen networks are used to improve the wound microenvironment and thus favour the regeneration of new tissue. The rationale behind the use of these materials is the approach that they present inherent natural cell-recognition domains and thus stimulate fibroblast and keratinocyte adhesion and proliferation in the wound bed [28]. In addition, such natural fibrillar collagen scaffolds are thought to provide guiding ridges for invading cells and thus may promote structured dermal wound healing.

Bovine collagen for biomedical applications is typically derived from processing and purifying bovine skin and tendons. The resulting collagen dispersion, consisting of native collagen fibrils, resembles dermis structurally and functionally to a high degree [43, 44] which promotes structured wound healing. These collagens are also highly conserved between different species [45, 46], e.g., the sequence correlation between human and bovine collagen I is 88%. This is consistent with the fact that a stable collagen triple helix structure allows only very little variation in the primary amino acid sequence without pivotal loss of structure and function [47]. Thus, molecular sequence variation between species is highly conserved, making bovine collagen suitable for use in humans.

Collagen-containing wound dressings may become integrated into the wound bed as a solid collagen gel. This gel may be partly consumed by proteolytic enzymes that are present in the fluid secreted during wound healing. Thus, a new collagen dressing can be placed on top of the remaining one when taking care of the wound. Hence, collagen is “fed” into the wound and will gradually be turned over and replaced by human dermis.

There remains a need for development of improved bioactive peptides that are capable of achieving improved wound healing and antimicrobial activity such as bioactive collagen VI peptides in biomaterials, such as medical devices, implants, wound care products and materials for use in the same.

### SUMMARY OF THE INVENTION

Here, the inventors have shown for the first time that the combination of two bioactive peptides from the alpha-3 chain of collagen VI greatly enhances wound healing and antimicrobial activity, as compared the individual peptides alone, even at comparable doses. By “comparable doses” we mean that the peptides tested individually were at the same dose as the two combined peptides; e.g. peptides were tested alone at a concentration of 3 μM, but were combined as two peptides at a concentration of 1.5 μM each for a total of a 3 μM administration of peptides. This effect will result in a pronounced potential to provide a versatile, multifunctional, and appropriate extracellular environment, able to actively contrast the onset of infections and inflammation, while promoting tissue regeneration and scar remodeling, and consequently deliver the desired enhancement in biocompatibility.

Furthermore, as described in Punjataewakupt et al., 2019, many known antiseptic agents suffer from cytotoxic effects and from bacterial resistance developing to them. In contrast, the peptides of the invention exhibit limited/reduced cytotoxicity and an inherent lower degree of development of multidrug resistance than synthetic antibiotic agents. In one embodiment the peptides described herein exhibit no cytotoxicity. Interestingly, the combination of two bioactive peptides is significantly improved in non-infected and infected wound healing scenarios. This demonstrates that the improved wound healing and antimicrobial activity of the peptide combination is not dependent on reducing or preventing an underlying infection.

A first aspect of the invention provides a composition comprising a combination of polypeptides derived from collagen type VI. For example, the composition may comprise at least two polypeptides derived from collagen type VI.

In one embodiment, the composition comprises:
(a) a collagen type VI polypeptide (i.e. a first polypeptide) comprising or consisting of an amino acid sequence derived from collagen type VI, or a fragment, variant, fusion or derivative thereof, or a fusion of said fragment, variant of derivative thereof, having the primary activity of being capable of promoting wound healing; and
(b) a collagen type VI polypeptide (i.e. a second polypeptide) comprising or consisting of an amino acid sequence derived from collagen type VI, or a fragment, variant, fusion or derivative thereof, or a fusion of said fragment, variant of derivative thereof, having the primary activity of being capable of exerting an antimicrobial effect.

Accordingly, in one embodiment, the composition comprises:
(a) a collagen type VI polypeptide (i.e. a first polypeptide) comprising or consisting of an amino acid sequence derived from collagen type VI, or a fragment, variant, fusion or derivative thereof, or a fusion of said fragment, variant of derivative thereof, wherein the first polypeptide is capable of promoting wound healing; and
(b) a collagen type VI polypeptide (i.e. a second polypeptide) comprising or consisting of an amino acid sequence derived from collagen type VI, or a fragment, variant, fusion or derivative thereof, or a fusion of said fragment, variant of derivative thereof, wherein the second polypeptide is capable of exerting an antimicrobial effect.

Thus, in one embodiment the composition has the dual effect of being capable of promoting wound healing and also being capable of exerting an antimicrobial effect.

It will be appreciated by the skilled person that such an effect may be synergistic—i.e. the effect of the combination of polypeptides may be greater than an additive effect of combining the two polypeptides.

It will be appreciated by persons skilled in the art that the collagen type VI may be from a human or non-human source. For example, the collagen type VI may be derived (directly or indirectly) from a non-human mammal, such as an ape (e.g. chimpanzee, bonobo, gorilla, gibbon and orangutan), monkey (e.g. macaque, baboon and colobus), rodent (e.g. mouse, rat) or ungulates (e.g. pig, horse and cow). The collagen VI may also be derived from birds, e.g. chicken (Gallus gallus).

Thus, by “collagen type VI” (also “collagen VI”) we include naturally occurring human collagen type VI, collagen type VI monomers, dimers and tetramers, collagen type VI microfibrils, and homologues thereof, such as bovine collagen type VI. We also include recombinant expression of human collagen VI and/or parts thereof, where the expressed molecules can be produced from human and/or non-human gene sequences. Such expression can make use of bacterial and/or human and/or non-human cellular expression systems (e.g. yeast expression systems). We also include synthetic cellular expression systems. We also include synthetic chemical synthesis of protein sequences from collagen VI and/or parts thereof.

Collagen type VI is typically comprised of each of three collagen VI peptide chains α1(VI), α2(VI) and α3(VI)). In some cases, the α3(VI) chain may be substituted for the α4(VI), α5(VI) or α6(VI) chain.

The sequences of the different collagen VI alpha chains are publicly available, such as at UniProt (https://www.uniprot.org/). Details of the UniProt ID for each alpha chain and the individual pages at UniProt are as follows:
Alpha-1: https://www.uniprot.org/uniprot/P12109
Alpha-2: https://www.uniprot.org/uniprot/P12110
Alpha-3: https://www.uniprot.org/uniprot/P12111
Alpha-4: https://www.uniprot.org/uniprot/A2AX52
Alpha-5: https://www.uniprot.org/uniprot/A8TX70
Alpha-6: https://www.uniprot.org/uniprot/A6NMZ7

Therefore, in certain embodiments, the collagen VI of the composition of the invention comprises or consists of any three amino acid chains selected from the group consisting of α1(VI), α2(VI), α3(VI), α4(VI), α5(VI) and α6(VI). In one embodiment the collagen VI comprises or consists of an α1(VI) chain, an α2(VI) chain and/or an α3(VI) chain.

In one embodiment, the polypeptides of the composition of the first aspect are, or are derived from, the α1, α2 and/or α3 chain of collagen type VI.

In one embodiment, the polypeptides of the composition of the first aspect are, or are derived from, a collagen VI α3(VI) chain.

In another embodiment, the collagen VI may comprise one α1(VI) chain, one α2(VI) chain and further comprises a third chain that is either an α3, α4, α5 or α6 chain.

In certain embodiments, the collagen VI is, or is a peptide derived from, human collagen VI. In an alternative embodiment the collagen VI is, or is a peptide derived, from bovine collagen VI.

In one embodiment, at least one of the polypeptides comprise or consist of an amino acid sequence derived from collagen type VI, or a fragment, variant, fusion or derivative thereof, or a fusion of said fragment, variant of derivative thereof, is derived from the collagen VI α3 chain, optionally wherein the polypeptides comprise or consist of different amino acid sequences. The amino acid sequences of the polypeptides may be overlapping or non-overlapping.

By “at least one of the polypeptides” we mean the first polypeptide, or the second polypeptide, or both the first and second polypeptides.

In one embodiment, the polypeptides comprising or consisting of an amino acid sequence derived from collagen type VI, or a fragment, variant, fusion or derivative thereof, or a fusion of said fragment, variant of derivative thereof, comprises or consists of one or more polypeptides selected from the group consisting of α1(VI), α2(VI) and α3(VI).

In one embodiment, the composition comprises one or more polypeptides selected from the group consisting of: collagen VI, collagen VI α1 chain, collagen VI α2 chain, collagen VI α3 chain, GVR28, FYL25, FFL25, VTT30, and SFV33.

A “polypeptide” is used herein in its broadest sense to refer to a compound of two or more subunit amino acids, amino acid analogues, or other peptidomimetics. The term “polypeptide” thus includes short peptide sequences and also longer polypeptides and proteins. As used herein, the term “amino acid” refers to either natural and/or unnatural or synthetic amino acids, including both D or L optical isomers, and amino acid analogues and peptidomimetics.

By an amino acid sequence “derived from” collagen type VI and/or the α1, α2 and/or α3 chain of collagen type VI we include amino acid sequences found within the amino acid sequence of a naturally occurring collagen type VI protein and/or the α1, α2 and/or α3 chain of collagen type VI. In particular, we include amino acid sequences that comprise at least five contiguous amino acids from the sequence of a naturally occurring collagen type VI and/or the α1, α2 and/or α3 chain of collagen type VI. For example, in one embodiment the amino acid sequence may contain at least 5 contiguous amino acids from collagen type VI and/or the α1, α2 and/or α3 chain of collagen type VI, for example at least 6, 7, 8, 9, 10, 11, 12, 13, 14, 15, 16, 17, 18, 19, 20, 21, 22, 23, 24, 25, 30, 40, 50 or more contiguous amino acids from collagen type VI and/or the α1, α2 and/or α3 chain of collagen type VI. Thus, the amino acid sequence derived from collagen type VI and/or the α1, α2 and/or α3 chain of collagen type VI corresponds to a fragment of collagen type VI and/or the α1, α2 and/or α3 chain of collagen type VI. Thus in one embodiment, the amino acid sequence derived from collagen type VI and/or the α1, α2 and/or α3 chain of collagen type VI is not the full-length sequence of collagen type VI and/or the full-length sequence of the α1, α2 and/or α3 chain of collagen type VI. Alternatively, or additionally, the amino acid sequence may not contain a sequence of contiguous amino acids from the full length of collagen type VI and/or the α1, α2 and/or α3 chain of collagen type VI. For example, the amino acid sequence may not contain at least 2, 3, 4, 5, 6, 7, 8, 9, 10, 11, 12, 13, 14, 15, 16, 17, 18, 19, 20, 21, 22, 23, 24, 25, 30, 40, 50 or more contiguous amino acids from collagen type VI and/or the α1, α2 and/or α3 chain of collagen type VI.

It will be clear to the skilled person that the first collagen type VI polypeptide and the second collagen type VI polypeptide are different polypeptides. i.e. the first collagen type VI polypeptide that promotes wound healing is a different polypeptide to the second type VI polypeptide that exerts an antimicrobial effect. By “different polypeptides” we include the meaning of having different amino acid sequences and/or different lengths of sequences, i.e. the peptides do not have identical sequences.

In one embodiment, at least one of the collagen type VI polypeptides, (or fragment, variant, fusion or derivative thereof of collagen VI) of the composition is capable of exerting an antimicrobial effect. Thus in one embodiment at least one of the polypeptides of the composition is capable of killing or attenuating the growth of microorganisms.

The antimicrobial properties of collagen VI and polypeptides comprising an amino acid sequence derived from collagen VI, such as GVR28, FYL25, FFL25, VTT30, and SFV33, are described in WO 2017/125585, the content of which is incorporated herein by reference.

By “capable of killing or attenuating the growth of microorganisms” we include collagen VI and polypeptides with antimicrobial activity. The antimicrobial activity may be in whole or in part, and may be dose dependent. This may be demonstrated by, for example, radial diffusion assays.

The microorganisms against which the polypeptides of the invention are efficacious may be selected from the group consisting of bacteria, mycoplasmas, yeasts, fungi and viruses. Thus, in one embodiment at least one of the polypeptides of the composition is capable of exerting an antibacterial effect.

In one embodiment, at least one of the polypeptides of the composition of the first aspect is/are capable of binding to the membrane of the microorganism. In another embodiment, at least one of the polypeptides may have affinity for negatively charged surfaces, for example a bacterial membrane. This affinity may be tested by, for example, affinity to heparin, wherein higher affinity to heparin indicates higher affinity to negatively charged surfaces.

Advantageously, the affinity or binding capability of at least one of the polypeptides is/are comparable to or greater than that of LL-37. Thus, in one embodiment, at least one of the polypeptides is/are capable of exhibiting an antimicrobial effect greater than or equal to that of LL-37 (i.e. LLGDFFRKSKEKIGKEFKRIVQRIKDFLRNLVPRTES; SEQ ID NO: 24). In some embodiments, at least one of the polypeptides is/are capable of exhibiting at least 5%, 10%, 15%, 20%, 25%. 30%, 40%, 50%, 60%, 70%, 80%, 90% or 100% better antimicrobial effect than LL-37, or a range selected between these specified percentages (for example, between 5-100%, 10-20%, 20-60% and so on).

In a preferred embodiment, at least one of the polypeptides is capable of exhibiting at least a 10% better antimicrobial effect than LL-37. In one embodiment at least one of the polypeptides is capable of exhibiting an antimicrobial effect in the range of 10-20% better than the antimicrobial effect of LL-37.

Thus, in one embodiment, at least one of the polypeptides is/are capable of exhibiting a wound healing effect greater than or equal to that of LL-37 (i.e. LLGDFFRKSKEKIGKEFKRIVQRIKDFLRNLVPRTES; SEQ ID NO: 24). In some embodiments, at least one of the polypeptides is/are capable of exhibiting at least 5%, 10%, 15%, 20%, 25%. 30%, 40%, 50%, 60%, 70%, 80%, 90% or 100% better wound healing effect than LL-37, or a range selected between these specified percentages (for example, between 5-100%, 10-20%, 20-60% and so on). In a preferred embodiment, at least one of the polypeptides is capable of exhibiting at least a 15% better wound healing effect than LL-37. In one embodiment at least one of the polypeptides is capable of exhibiting a wound healing effect in the range of 15-25% better than the wound healing effect of LL-37.

In one embodiment, at least one of the polypeptides is/are capable of causing structural alterations to the microorganism, including, for example, membrane perturbations, blebbing or exudation of cytoplasmic constituents.

Thus, at least one of the polypeptides of the composition may be capable of causing disruption of the membrane of the microorganisms. This can, for example, be quantified through microscopy studies, such as electron microscopy or fluorescence microscopy, studying the uptake of fluorescent molecules by the microorganisms.

In a further embodiment, at least one of the polypeptides of the composition may be capable of promoting wound closure and/or wound healing.

By “promoting wound closure” and/or “wound healing” we include aiding the healing process of the wound, for example by accelerating healing. For example, the wound care product may be capable of enhancing epithelial regeneration and/or healing of wound epithelia and/or wound stroma. In one embodiment, the wound care product may be capable of enhancing the proliferation of epithelial and/or stromal cells through a non-lytic mechanism. The wound closing capability may be quantified by, for example, cell scratch experiments and in vivo experiments in murine or porcine models. By “enhancing epithelia regeneration” we include the enhancement of epidermal regeneration or, in other words, the enhancement of the regeneration of the epidermis. By “enhancing healing of wound epithelia” we include the enhancement of epidermal healing or, in other words, the enhancement of the healing of the epidermis (e.g. the wounded epidermis). By “enhancing healing of wound stroma” we include the enhancement of dermal healing or, in other words, the enhancement of the healing of the dermis (e.g. the wounded dermis).

Thus, at least one of the polypeptides of the composition may have a role in wound care by promoting wound closure/healing and/or by preventing infection of a wound.

In a further embodiment, at least one of the polypeptides may have antimicrobial activity and at least one of the polypeptides (either the same or a different polypeptide) may promote wound healing. For example, at least one of the polypeptides may have antimicrobial activity but does not promote wound healing alone. Alternatively, or additionally, at least one of the polypeptides may promote wound healing but has no antimicrobial activity alone. Accordingly, the properties of polypeptides may be mutually exclusive to the polypeptides in the composition. Where polypeptides are capable of both activities, one of the activities of said polypeptide may be considered its primary activity and the other property its secondary activity. For example, the primary activity of at least one of the polypeptides may be antimicrobial activity and the second activity may be the promotion of wound healing. Alternatively, polypeptides may be considered to only have a primary activity. In some embodiments, a first polypeptide has wound healing and antimicrobial properties (e.g. GVR28) and a second polypeptide has no wound healing properties but has antimicrobial properties (e.g. SFV33), optionally wherein the second polypeptide has better antimicrobial properties than the first polypeptide.

The wounds to be treated by the composition of the first aspect may be extracorporeal (i.e. surface wounds of the skin and underlying tissue) and/or intracorporeal (such as internal wounds due to organ transplantation or removal of tissue/parts of organs, e.g. following colon surgery).

It will be appreciated by persons skilled in the art that the composition of the first aspect may exert an antimicrobial effect against Gram-positive and/or Gram-negative bacteria. For example, the microorganisms may be bacteria selected from the group consisting of: Pseudomonas aeruginosa, Staphylococcus aureus, Escherichia coli, group A streptococcus (e.g. Streptococcus pyogenes), group B streptococcus e.g. Streptococcus agalactiae), group C streptococcus (e.g. Streptococcus dysgalactiae), group D streptococcus (e.g. Entero-coccus faecalis), group F streptococcus (e.g. Streptococcus anginosus), group G streptococcus (e.g. Streptococcus dysgalactiae equisimilis), alpha-hemolytic streptococcus (e.g. Streptococcus viridans, Streptococcus pneumoniae), Streptococcus bovis, Streptococcus mitis, Streptococcus anginosus, Streptococcus sanguinis, Streptococcus suis, Streptococcus mutans, Moraxella catarrhalis, Non-typeable Haemophilus influenzae (NTHi), Haemophilus influenzae b (Hib), Actinomyces naeslundii, Fusobacterium nucleaturn, Prevotella intermedia, Klebsiella pneumoniae, Enterococcus cloacae, Enterococcus faecalis, Staphylococcus epidermidis, multidrug-resistant Pseudomonas aeruginosa (MRPA), multidrug-resistant Staphylococcus aureus (MRSA), multidrug-resistant Escherichia coli (MREC), multidrug-resistant Staphylococcus epidermidis (MRSE), multidrug-resistant Klebsiella pneumoniae (MRKP), multidrug-resistant Entercoccus faecium (MREF), multidrug-resistant Acinetobacter baumannii (MRAB) and multidrug-resistant Enterobacter spp. (MRE).

In one embodiment the microorganisms are bacteria which are resistant to one or more conventional antibiotic agents.

By “conventional antibiotic agent” we include known agents that are capable of killing and/or attenuating the growth of microorganisms, for example natural and synthetic penicillins and cephalosporins, sulphonamides, erythromycin, kanomycin, tetracycline, chloramphenicol, rifampicin and including gentamicin, ampicillin, benzypenicillin, benethamine penicillin, benzathine penicillin, phenethicillin, phenoxy-methyl penicillin, procaine penicillin, cloxacillin, flucloxacillin, methicillin sodium, amoxicillin, bacampicillin hydrochloride, ciclacillin, mezlocillin, pivampicillin, talampicillin hydrochloride, carfecillin sodium, piperacillin, ticarcillin, mecillinam, pirmecillinan, cefaclor, cefadroxil, cefotaxime, cefoxitin, cefsulodin sodium, ceftazidime, ceftizoxime, cefuroxime, cephalexin, cephalothin, cephamandole, cephazolin, cephradine, latamoxef disodium, aztreonam, chlortetracycline hydrochloride, clomocycline sodium, demeclocydine hydrochloride, doxycycline, lymecycline, minocycline, oxytetracycline, amikacin, framycetin sulphate, neomycin sulphate, netilmicin, tobramycin, colistin, sodium fusidate, polymyxin B sulphate, spectinomycin, vancomycin, calcium sulphaloxate, sulfametopyrazine, sulphadiazine, sulphadimidine, sulphaguanidine, sulphaurea, capreomycin, metronidazole, tinidazole, cinoxacin, ciprofloxacin, nitrofurantoin, hexamine, streptomycin, carbenicillin, colistimethate, polymyxin B, furazolidone, nalidixic acid, trimethoprim-sulfamethox-azole, clindamycin, lincomycin, cycloserine, isoniazid, ethambutol, ethionamide, pyrazinamide and the like; anti-fungal agents, for example miconazole, ketoconazole, itraconazole, fluconazole, amphotericin, flucytosine, griseofulvin, natamycin, nystatin, and the like; and anti-viral agents such as acyclovir, AZT, ddI, amantadine hydrochloride, inosine pranobex, vidarabine, and the like.

Thus, in one embodiment, the microorganism is selected from the group consisting of: multidrug-resistant Staphylococcus aureus (MRSA) (methicillin resistant Staphylococcus aureus), multidrug-resistant Pseudomonas aeruginosa (MRPA), multidrug-resistant Escherichia coli (MREC), multidrug-resistant Staphylococcus epidermidis (MRSE) and multidrug-resistant Klebsiella pneumoniae (MRKP).

Advantageously, the composition according to the first aspect of the invention exhibits selective toxicity to microbial agents. By ‘selective’ we mean the polypeptide of the composition is preferentially toxic to one or more microorganisms (such as bacteria, mycoplasmas, yeasts, fungi and/or viruses) compared to mammalian, e.g. human, host cells. For example, the toxicity of the polypeptide of the composition to a target microorganism is at least two-fold greater than the toxicity of that composition to mammalian cells, more preferably at least three-fold, at least four-fold, at least five-fold, at least six-fold, at least eight-fold, at least ten-fold, at least fifteen-fold or at least twenty-fold.

Conveniently, at least one of the polypeptides of the composition of the first aspect is substantially non-toxic to mammalian, e.g. human, cells. In some embodiments, the antimicrobial effect is specific to Gram-positive and/or Gram-negative bacteria and is inactive to other organisms (e.g. mycoplasma, yeast, fungus and/or viruses).

For example, at least one of the polypeptides of the composition of the first aspect may not exhibit cytotoxicity to erythrocytes or monocytes at concentrations at concentrations which can be used to kill microorganisms such as bacteria. In one embodiment at least one of the polypeptides of the composition does not exhibit cytotoxicity at a concentration of up to 30 μM, or alternatively at a concentration of up to 50 μM.

In this way, when the compounds are used to treat microbial infections, for example, dosing regimens can be selected such that microbial cells are destroyed with minimal damage to healthy host tissue. Thus, at least one of the polypeptides of the composition may exhibit a ‘therapeutic window’.

In one embodiment, at least one of the polypeptides of the composition is capable of exerting an anti-endotoxic effect.

By “anti-endotoxic effect” we include polypeptides which counteract the effects induced by endotoxins. For example, in one embodiment at least one of the polypeptides of the composition is capable of suppressing, at least in part, LPS induction of nitrite.

In one embodiment, at least one of the polypeptides of the composition is derived from or shows amino acid sequence homology to a VWA domain, for example a globular VWA domain. Thus, a polypeptide of the composition may comprise or consist of an amino acid sequence which corresponds to at least five (for example, at least 10, 15, 20 or more) contiguous amino acids of a VWA domain, or an amino acid sequence which has at least 70% (for example at least 80%, 90% or 95%) identity with such as sequence.

In a further embodiment, a polypeptide of the composition may comprise or consist of an intact VWA domain.

By “VWA domain” we include the type A domains of von Willebrand factor, and domains showing homology to the type A domains of von Willebrand factor, as well as VWA-domain containing regions.

In one embodiment, a polypeptide of the composition is derived from the α3 chain of collagen type VI. Thus, a polypeptide of the composition may be derived from the α 3N or α3C regions. For example, a polypeptide of the composition may be or may be derived from the N2, N3 or C1 domain of the α3 chain of collagen type VI.

In an alternative embodiment, a polypeptide of the composition is derived from the α4 chain of collagen type VI.

In another alternative embodiment, a polypeptide of the composition is derived from the α5 chain of collagen type VI.

In a further alternative embodiment, a polypeptide of the composition is derived from the α6 chain of collagen type VI.

In a still further alternative embodiment, a polypeptide of the composition is derived from the α2 chain of collagen type VI, for example from the α2N region.

It will be appreciated by persons skilled in the art that at least one of the polypeptides of the composition of the first aspect may have cationic residues on their surface, or cationic sequence motifs therein.

Thus, in one embodiment, at least one of the polypeptides of the composition has a net positive charge. For example, the polypeptide may have a charge ranging from between +2 to +9.

In a further embodiment, at least one of the polypeptides of the composition has at least 30% hydrophobic residues.

In a still further embodiment, at least one of the polypeptides of the composition may have an amphipathic structure.

Exemplary collagen VI polypeptides of the composition of the first aspect of the invention comprise or consist of the amino acid sequence of any one of SEQ ID NOs: 1 to 23 (as shown in Table 1 below) or a fragment, variant, fusion or derivative thereof, or a fusion of said fragment, variant or derivative thereof, which retains an antimicrobial activity of any one of SEQ ID NOs:1 to 23. Each of SEQ ID NOs:1 to 23 may be combined in a composition with any one or more of SEQ ID NOs:1 to 23. For example, a composition may be formed of a polypeptide comprising or consisting of the amino acid sequence of SEQ ID NO:1 and a polypeptide comprising or consisting of the amino acid sequence of SEQ ID NO:2, 3, 4, 5, 6, 7, 8, 9, 10, 11, 12, 13, 14, 15, 16, 17, 18, 19, 20, 21, 22 and/or 23. Alternatively, a composition may be formed of a polypeptide comprising or consisting of the amino acid of SEQ ID NO:5 and a polypeptide comprising or consisting of the amino acid of SEQ ID NO:1, 2, 3, 4, 6, 7, 8, 9, 10, 11, 12, 13, 14, 15, 16, 17, 18, 19, 20, 21, 22 and/or 23. In some embodiments, the polypeptide is not SEQ ID NO: 6.

Table 1 below shows various polypeptides of the composition of the invention. Of these wound healing data is available for SEQ ID NOs: 1 to 6, and GVR28, FYL25, FFL25 and VTT30 (SEQ ID NOs: 1 to 4) have wound healing properties. Wound healing data is not available for SEQ ID NOs: 7 to 23. SEQ ID NOs 1 to 5 and 7 to 23 all have antimicrobial activity. Thus, SEQ ID NOs 1 to 4 (GVR28, FYL25, FFL25 and VTT30) have dual activities—i.e. they are capable of both exerting a wound healing effect and exerting an antimicrobial effect.

TABLE 1
Exemplary polypeptides of the composition of the invention

| | | | Anti- | Wound | |
|---|---|---|---|---|---|
| | | | microbial | healing | |
| Peptide | Sequence | MW | activity? | activity? | Charge |
| GVR28 | GVRPDGFAHIRDFVSRIVRRLNIGP | 3,163 | Yes | Yes | 4 |
| | SKV [SEQ ID NO: 1] | | | | |
| FYL25 | FYLKTYRSQAPVLDAIRRLRLRGGS | 2,937 | Yes | Yes | 5 |
| | [SEQ ID NO: 2] | | | | |
| FFL25 | FFLKDFSTKRQIIDAINKVVYKGGR | 2,944 | Yes | Yes | 4 |
| | [SEQ ID NO: 3] | | | | |
| VTT30 | VTTEIRFADSKRKSVLLDKIKNLQV | 3,403 | Yes | Yes | 4 |
| | ALTSK [SEQ ID NO: 4] | | | | |
| SFV33 | SFVARNTFKRVRNGFLMRKVAVFFS | 3,803 | Yes | No | 7 |
| | NTPTRASP [SEQ ID NO: 5] | | | | |
| DVN32 | DVNVFAIGVEDADEGALKEIASEPL | 3,490 | No | No | −6 |
| | NMHMFNL [SEQ ID NO: 6] | | | | |
| KPE20 | KPEILNLVKRMKIKTGKALN | 2,295 | Yes | Unknown | 5 |
| | [SEQ ID NO: 7] | | | | |
| GFA20 | GFAHIRDFVSRIVRRLNIGP | 2,324 | Yes | Unknown | 5 |
| | [SEQ ID NO: 8] | | | | |
| QAP20 | QAPVLDAIRRLRLRGGSPLN | 2,203 | Yes | Unknown | 3 |
| | [SEQ ID NO: 9] | | | | |
| KGF20 | KGFESKVDAILNRISQMHRV | 2,329 | Yes | Unknown | 3 |
| | [SEQ ID NO: 10] | | | | |
| RKV20 | RKVAVFFSNTPTRASPQLRE | 2,305 | Yes | Unknown | 2 |
| | [SEQ ID NO: 11] | | | | |
| VAA20 | VAAKPVATKMAVRPPVAVKP | 2,031 | Yes | Unknown | 3 |
| | [SEQ ID NO: 12] | | | | |
| AAK20 | AAKPVATKPEVPRPQAAKPA | 2,027 | Yes | Unknown | 4 |
| | SEQ ID NO: 13] | | | | |
| TTK20 | TTKPVTTTKPVTTTTKPVTT [SEQ | 2,103 | Yes | Unknown | 3 |
| | ID NO: 14] | | | | |
| AAA76 | AAAKPAPAKPVAAKPVATKMATVRP | 7,324 | Yes | Unknown | 13 |
| | PVAVKPATAAKPVAAKPAAVRPPAA | | | | |
| | AAAKPVATKPEVPRPQAAKPAATKP | | | | |
| | A | | | | |
| | [SEQ ID NO: 15] | | | | |
| TSS36 | TSSPTSNPVTTTKPVTTTKPVTTTTK | 3,703 | Yes | Unknown | 4 |
| | PVTTTTKPVT | | | | |
| | [SEQ ID NO: 16] | | | | |
| YDR20 | YDRLIKESRRQKTRVFAVVI | 2 473 | Yes | Unknown | 4 |
| | [SEQ ID NO: 17] | | | | |
| EQN20 | EQNFHKARRFVEQVARRLTL | 2 499 | Yes | Unknown | 3 |
| | [SEQ ID NO: 18] | | | | |
| VVH20 | VVHAINAIVRSPRGGARRHA | 2 137 | Yes | Unknown | 4 |
| | [SEQ ID NO: 19] | | | | |
| LRL20 | LRLKPYGALVDKVKSFTKRF | 2 367 | Yes | Unknown | 5 |
| | [SEQ ID NO: 20] | | | | |
| FTK20 | FTKRFIDNLRDRYYRCDRNL | 2 665 | Yes | Unknown | 3 |
| | [SEQ ID NO: 21] | | | | |
| RDA20 | RDALKSSVDAVKYFGKGTYT | 2 206 | Yes | Unknown | 2 |
| | [SEQ ID NO: 22] | | | | |
| TKR20 | TKRFAKRLAERFLTAGRTDP | 2 335 | Yes | Unknown | 4 |
| | [SEQ ID NO: 23] | | | | |

In some embodiments, the polypeptide with antimicrobial properties is selected from the group consisting of GVR28, SFV33, FYL25, FFL25, VTT30, KPE20, GFA20, AAA76, YDR20, EQN20, VVH20, LRL20, FTK20, RDA20 and TKR20. In some embodiments, the polypeptide with antimicrobial properties is not AAK20, KGF20, QAP20, RKV20, TSS36, TTK20, VAA20 or DVN32.

In some embodiments, the polypeptide with wound healing properties is selected from the group consisting of GVR28, SFV33, FYL25 and FFL25.

For example, the polypeptides of the composition of the invention may comprise or consist of an amino acid sequence selected from the group consisting of SEQ ID NOs: 1 to 5:

″GVR28″:
[SEQ ID NO: 1]
GVRPDGFAHIRDFVSRIVRRLNIGPSKV
″FYL25″:
[SEQ ID NO: 2]
FYLKTYRSQAPVLDAIRRLRLRGGS
″FFL25″:
[SEQ ID NO: 3]
FFLKDFSTKRQIIDAINKVVYKGGR
″VTT30″:
[SEQ ID NO: 4]
VTTEIRFADSKRKSVLLDKIKNLQVALTSK
″SFV33″:
[SEQ ID NO: 5]
SFVARNTFKRVRNGFLMRKVAVFFSNTPTRASP

or a fragment, variant, fusion or derivative thereof, or a fusion of said fragment, variant or derivative thereof, which retains an antimicrobial activity of any one of SEQ ID NOs: 1 to 5, or which retains a wound healing promoting activity of any one of SEQ ID NOs: 1 to 4.

Therefore, in one embodiment the composition of the present invention comprises a polypeptide comprising or consisting of the amino acid sequence according to SEQ ID NO:1 (i.e. GVR28), or fragments, variants, fusions or derivatives thereof and fusions of said fragments, variants and derivatives thereof which retain the wound healing activity of SEQ ID NO: 1.

In one embodiment, the composition of the present invention comprises a polypeptide comprising or consisting of the amino acid sequence according to SEQ ID NO:5 (i.e. SFV33), or fragments, variants, fusions or derivatives thereof, and fusions of said fragments, variants and derivatives thereof, which retain an antimicrobial activity of SEQ ID NO: 5.

In a particularly preferred embodiment, the composition of the present invention comprises a polypeptide (i.e. a first polypeptide) comprising or consisting of the amino acid sequence of SEQ ID NO:1 (GVR28), and a polypeptide (i.e. a second polypeptide) comprising or consisting of the amino acid sequence of SEQ ID NO:5 (SFV33), or a fragment, variant, fusion or derivative thereof, or a fusion of said fragment, variant or derivative thereof, which retains an antimicrobial activity of either one of SEQ ID NO:1 or 5, respectively.

In such a composition, the primary action of GVR28 (SEQ ID NO: 1) is promoting wound healing, and the primary action of SFV33 (SEQ ID NO: 5) is providing/exerting an antimicrobial effect.

Accordingly, in one embodiment the composition of the present invention comprises or consists of:
(a) A first polypeptide comprising or consisting of the amino acid sequence of SEQ ID NO:1; and
(b) A second polypeptide comprising or consisting of the amino acid sequence of SEQ ID NO: 5.

Such peptides may also have a secondary effect. For example, although GVR28 has a primary action of promoting wound healing, GVR28 may also have a secondary activity wherein the peptide is also capable of exerting an antimicrobial effect.

The composition of the present invention may comprise or consist of two or more polypeptides according to SEQ ID NOs:1 to 23 in equal doses, for example at a ratio of 1:1. Alternatively, each polypeptide in the composition may be a different dose, for example, 0.5:1, 0.6:1, 0.7:1, 0.8:1, 0.9:1. In some embodiments, the composition comprises two polypeptides, wherein the first polypeptide is GVR28 (SEQ ID NO: 1) and the second polypeptide is SFV33 (SEQ ID NO: 5) at a ratio of 0.5:1, 0.6:1, 0.7:1, 0.8:1, 0.9:1 or 1:1 (either as GVR28:SFV33 or as SFV33:GVR28). In some embodiments, the composition comprises two polypeptides, wherein the first polypeptide is GVR28 and the second polypeptide is SFV28 at a ratio of 1:0.9, respectively. In some embodiments, the composition comprises two polypeptides, wherein the first polypeptide is GVR28 and the second polypeptide is SFV28 at a ratio of 1:1. In some embodiments, the polypeptide is not SEQ ID NO: 6.

Exemplary compositions of the present invention have improved wound healing compared with a control treatment, such as compared with a carrier only control in which polypeptides of the present invention are not present. For example, the wound healing of the polypeptides of the composition may be at least two-fold greater than the wound healing of a carrier only control, more preferably at least three-fold, at least four-fold, at least five-fold, at least six-fold, at least eight-fold, at least ten-fold, at least fifteen-fold or at least twenty-fold improved.

It will be appreciated by persons skilled in the art that the term “amino acid”, as used herein, includes the standard twenty genetically-encoded amino acids and their corresponding stereoisomers in the ‘D’ form (as compared to the natural ‘L’ form), omega-amino acids other naturally-occurring amino acids, unconventional amino acids (e.g., α,α-disubstituted amino acids, N-alkyl amino acids, etc.) and chemically derivatised amino acids (see below).

When an amino acid is being specifically enumerated, such as ‘alanine’ or ‘Ala’ or ‘A’, the term refers to both L-alanine and D-alanine unless explicitly stated otherwise. Other unconventional amino acids may also be suitable components for polypeptides of the present invention, as long as the desired functional property is retained by the polypeptide. For the peptides shown, each encoded amino acid residue, where appropriate, is represented by a single letter designation, corresponding to the trivial name of the conventional amino acid.

In one embodiment, at least one of the polypeptides derived from collagen type VI of the composition of the first aspect comprise or consist of L-amino acids.

Where at least one of the polypeptides comprises an amino acid sequence according to a reference sequence (for example, SEQ ID NOs: 1 to 23), it may comprise additional amino acids at its N- and/or C-terminus beyond those of the reference sequence, for example, at least one of the polypeptides may comprise additional amino acids at its N-terminus. Likewise, where at least one of the polypeptides comprises a fragment, variant or derivative of an amino acid sequence according to a reference sequence, it may comprise additional amino acids at its N- and/or C-terminus.

In addition, where the composition comprises collagen type VI, one or more of the collagen type VI α chains may comprise additional amino acids at its N- and/or C-terminus, for example, the polypeptide may comprise additional amino acids at its N-terminus.

In one embodiment, at least one of the polypeptides of the composition comprises or consists of a fragment of the amino acid sequence according to a reference sequence (for example, a fragment of any one of SEQ ID NOs: 1 to 23). Thus, at least one of the polypeptides may comprise or consist of at least 5 contiguous amino acid of the reference sequence, for example at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74 or 75 contiguous amino acids, e.g. of any one of SEQ ID NOs: 1 to 23. In some embodiments, the fragment of the polypeptide is not a fragment of SEQ ID NO: 6.

It will be further appreciated by persons skilled in the art that at least one of the polypeptides of the composition of the first aspect may comprise or consist of a variant of the amino acid sequence according to a reference sequence (for example, a variant of any one of SEQ ID NOs: 1 to 23), or fragment of said variant. Such a variant may be non-naturally occurring. In some embodiments, the variant is not a variant of SEQ ID NO: 6.

By “variants” of the polypeptide we include insertions, deletions and substitutions, either conservative or non-conservative. For example, conservative substitution refers to the substitution of an amino acid within the same general class (e.g. an acidic amino acid, a basic amino acid, a non-polar amino acid, a polar amino acid or an aromatic amino acid) by another amino acid within the same class. Thus, the meaning of a conservative amino acid substitution and non-conservative amino acid substitution is well known in the art. In particular, we include variants of the polypeptide which exhibit an antimicrobial activity and/or promote wound healing. For example, a variant of a polypeptide that has a primary function of promoting wound healing (and/or antimicrobial activity) may be a variant that retains a primary function of promoting wound healing (and/or antimicrobial activity). A variant of a polypeptide may comprise at least 10%, 20%, 30%, 40%, 50%, 60%, 70%, 80%, 90%, 95%, 96%, 97%, 98%, 99% or 100% activity compared with the polypeptide from which the variant derives. Alternatively, the variant may have increased activity compared with the polypeptide from which the variant derives.

In one embodiment, the variant has an amino acid sequence which has at least 50% identity with the amino acid sequence according to a reference sequence (for example, SEQ ID NOs: 1 to 23) or a fragment thereof, for example at least 55%, 60%, 65%, 70%, 75%, 80%, 85%, 90%, 95%, 96%, 97%, 98% or at least 99% identity.

Any one or more of the polypeptides in the composition may be a variant. For example, a first polypeptide may correspond to a sequence according to SEQ ID NOs:1 to 23, and a second or further polypeptide may correspond to a variant of a sequence according to SEQ ID NOs:1 to 23. In some embodiments, the first and/or second polypeptides are not SEQ ID NO: 6 or variants thereof.

The percent sequence identity between two polypeptides may be determined using suitable computer programs, for example the GAP program of the University of Wisconsin Genetic Computing Group and it will be appreciated that percent identity is calculated in relation to polypeptides whose sequences have been aligned optimally.

The alignment may alternatively be carried out using the Clustal W program (as described in Thompson et al., 1994, Nuc. Acid Res. 22:4673-4680, which is incorporated herein by reference).

The parameters used may be as follows:
Fast pairwise alignment parameters: K-tuple(word) size; 1, window size; 5, gap penalty; 3, number of top diagonals; 5. Scoring method: x percent.
Multiple alignment parameters: gap open penalty; 10, gap extension penalty; 0.05.
Scoring matrix: BLOSUM.

Alternatively, the BESTFIT program may be used to determine local sequence alignments.

For example, in one embodiment, amino acids from the above reference sequences may be mutated in order to reduce proteolytic degradation of the polypeptide, for example by I,F to W modifications (see Strömstedt et al, Antimicrobial Agents Chemother 2009, 53, 593, which is incorporated herein by reference).

Variants may be made using the methods of protein engineering and site-directed mutagenesis well known in the art using the recombinant polynucleotides (see example, see Molecular Cloning: a Laboratory Manual, 3rd edition, Sambrook & Russell, 2000, Cold Spring Harbor Laboratory Press, which is incorporated herein by reference).

In a further embodiment, at least one of the polypeptides of the composition comprises or consists of an amino acid which is a species homologue of any one of the above amino acid sequences (e.g. SEQ ID NOS: 1 to 23). By “species homologue” we include that the polypeptide corresponds to the same amino acid sequence within an equivalent protein from a non-human species, i.e. which polypeptide exhibits the maximum sequence identity with of any one of SEQ ID NOS: 1 to 23 (for example, as measured by a GAP or BLAST sequence comparison). Typically, the species homologue polypeptide will be the same length as the human reference sequence (i.e. SEQ ID NOS: 1 to 23).

In a still further embodiment, at least one of the polypeptides of the composition comprises or consists of a fusion protein.

By “fusion” of a polypeptide we include an amino acid sequence corresponding to a reference sequence (for example, any one of SEQ ID NOs: 1 to 23, or a fragment or variant thereof) fused to any other polypeptide. For example, the said collagen VI or polypeptide may be fused to a polypeptide such as glutathione-S-transferase (GST) or protein A in order to facilitate purification of said polypeptide. Examples of such fusions are well known to those skilled in the art. Similarly, the said collagen VI or polypeptide may be fused to an oligo-histidine tag such as His6 or to an epitope recognised by an antibody such as the well-known Myc tag epitope. In addition, fusions comprising a hydrophobic oligopeptide end-tag may be used. Fusions to any variant or derivative of said collagen VI or polypeptide are also included in the scope of the invention. Additionally, a first polypeptide of the invention may be fused to a second polypeptide of the present invention, optionally wherein a linker is fused between each polypeptide. Thus, for example, a polypeptide comprising or consisting of SEQ ID NO: 1 (or variant, fragment or derivative thereof) may be fused to a polypeptide comprising or consisting of SEQ ID NO:5 (or variant, fragment or derivative thereof), or vice versa, optionally wherein a linker is present between the two polypeptides. It will be appreciated that fusions (or variants or derivatives thereof) which retain desirable properties, such as an antimicrobial activity and/or promotion of wound healing, are preferred.

A fusion may also comprise a polypeptide according to any one of SEQ ID NOs:1 to 23 fused to any other one or more polypeptide according to any one or more of SEQ ID NOs:1 to 23, optionally wherein the polypeptides are fused via a linker. In some embodiments, the fusion may be of multiple polypeptides but does not result in the full α3 chain of collagen VI. For example, a fusion polypeptide of any one or more of SEQ ID NOs:1 to 23 may be of an amino acid length less than that of full α3 chain of collagen VI. For example, the amino acid sequence of the fusion polypeptide may not contain at least 2, 3, 4, 5, 6, 7, 8, 9, 10, 11, 12, 13, 14, 15, 16, 17, 18, 19, 20, 21, 22, 23, 24, 25, 30, 40, 50 or more contiguous amino acids from the α3 chain of collagen type VI.

The fusion may comprise a further portion which confers a desirable feature on at least one of the said polypeptides of the composition of the invention; for example, the portion may be useful in detecting or isolating at least one of the polypeptides, or promoting cellular uptake of at least one of the polypeptides. The portion may be, for example, a biotin moiety, a streptavidin moiety, a radioactive moiety, a fluorescent moiety, for example a small fluorophore or a green fluorescent protein (GFP) fluorophore, as well known to those skilled in the art. The moiety may be an immunogenic tag, for example a Myc tag, as known to those skilled in the art or may be a lipophilic molecule or polypeptide domain that is capable of promoting cellular uptake of the polypeptide, as known to those skilled in the art.

It will be appreciated by persons skilled in the art that at least one of the polypeptides of the composition may comprise one or more amino acids that are modified or derivatised, for example by PEGylation, amidation, esterification, acylation, acetylation and/or alkylation. The moiety may be a tag selected from the group consisting of: FLAG tag, His tag, GST tag, MBP tag, Trx tag, NusA tag, SUMO tag, SET tag, DsbC tag, Skp tag, T7PK tag, GB1 tag, ZZ tag, Streptag II, HA tag, Softag 1, Softag 3, T7 tag, S tag, and mCherry tag.

As appreciated in the art, pegylated proteins may exhibit a decreased renal clearance and proteolysis, reduced toxicity, reduced immunogenicity and an increased solubility [Veronese, F. M. and J. M. Harris, Adv Drug Deliv Rev, 2002. 54(4): p. 453-6., Chapman, A. P., Adv Drug Deliv Rev, 2002. 54(4): p. 531-45] (incorporated herein by reference). Pegylation has been employed for several protein-based drugs including the first pegylated molecules asparaginase and adenosine deaminase [Veronese, F. M. and J. M. Harris, Adv Drug Deliv Rev, 2002. 54(4): p. 453-6., Veronese, F. M. and G. Pasut, Drug Discov Today, 2005. 10(21): p. 1451-8] (incorporated herein by reference).

In order to obtain a successfully pegylated protein, with a maximally increased half-life and retained biological activity, several parameters that may affect the outcome are of importance and should be taken into consideration. The PEG molecules may differ, and PEG variants that have been used for pegylation of proteins include PEG and monomethoxy-PEG. In addition, they can be either linear or branched [Wang, Y. S., et al., Adv Drug Deliv Rev, 2002. 54(4): p. 547-70] (incorporated herein by reference). The size of the PEG molecules used may vary and PEG moieties ranging in size between 1 and 40 kDa have been linked to proteins [Wang, Y. S., et al., Adv Drug Deliv Rev, 2002. 54(4): p. 547-70., Sato, H., Adv Drug Deliv Rev, 2002. 54(4): p. 487-504, Bowen, S., et al., Exp Hematol, 1999. 27(3): p. 425-32, Chapman, A. P., et al., Nat Biotechnol, 1999. 17(8): p. 780-3] (incorporated herein by reference). In addition, the number of PEG moieties attached to the protein may vary, and examples of between one and six PEG units being attached to proteins have been reported [Wang, Y. S., et al., Adv Drug Deliv Rev, 2002. 54(4): p. 547-70., Bowen, S., et al., Exp Hematol, 1999. 27(3): p. 425-32] (incorporated herein by reference). Furthermore, the presence or absence of a linker between PEG as well as various reactive groups for conjugation have been utilised. Thus, PEG may be linked to N-terminal amino groups, or to amino acid residues with reactive amino or hydroxyl groups (Lys, His, Ser, Thr and Tyr) directly or by using γ-amino butyric acid as a linker. In addition, PEG may be coupled to carboxyl (Asp, Glu, C-terminal) or sulfhydryl (Cys) groups. Finally, Gln residues may be specifically pegylated using the enzyme transglutaminase and alkylamine derivatives of PEG has been described [Sato, H., Adv Drug Deliv Rev, 2002. 54(4): p. 487-504] (incorporated herein by reference).

It has been shown that increasing the extent of pegylation results in an increased in vivo half-life. However, it will be appreciated by persons skilled in the art that the pegylation process will need to be optimised for a particular protein on an individual basis.

PEG may be coupled at naturally occurring disulphide bonds as described in WO 2005/007197, which is incorporated herein by reference. Disulfide bonds can be stabilised through the addition of a chemical bridge which does not compromise the tertiary structure of the protein. This allows the conjugating thiol selectivity of the two sulphurs comprising a disulfide bond to be utilised to create a bridge for the site-specific attachment of PEG. Thereby, the need to engineer residues into a peptide for attachment of to target molecules is circumvented.

A variety of alternative block copolymers may also be covalently conjugated as described in WO 2003/059973, which is incorporated herein by reference. Therapeutic polymeric conjugates can exhibit improved thermal properties, crystallisation, adhesion, swelling, coating, pH dependent conformation and biodistribution. Furthermore, they can achieve prolonged circulation, release of the bioactive in the proteolytic and acidic environment of the secondary lysosome after cellular uptake of the conjugate by pinocytosis and more favourable physicochemical properties due to the characteristics of large molecules (e.g. increased drug solubility in biological fluids). Block copolymers, comprising hydrophilic and hydrophobic blocks, form polymeric micelles in solution. Upon micelle disassociation, the individual block copolymer molecules are safely excreted.

Chemical derivatives of one or more amino acids may also be achieved by reaction with a functional side group. Such derivatised molecules include, for example, those molecules in which free amino groups have been derivatised to form amine hydrochlorides, p-toluene sulphonyl groups, carboxybenzoxy groups, t-butyloxycarbonyl groups, chloroacetyl groups or formyl groups. Free carboxyl groups may be derivatised to form salts, methyl and ethyl esters or other types of esters and hydrazides. Free hydroxyl groups may be derivatised to form O-acyl or O-alkyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g. acetylation or thioglycolic acid amidation), terminal carboxylamidation (e.g. with ammonia or methylamine), and the like terminal modifications.

It will be further appreciated by persons skilled in the art that peptidomimetic compounds may also be useful. Thus, by “collagen type VI polypeptide” we include peptidomimetic compounds which have an antimicrobial activity and/or which may be capable of promoting wound healing. The term “peptidomimetic” refers to a compound that mimics the conformation and desirable features of a particular peptide as a therapeutic agent.

For example, the polypeptides of the invention include not only molecules in which amino acid residues are joined by peptide (—CO—NH—) linkages but also molecules in which the peptide bond is reversed. Such retro-inverso peptidomimetics may be made using methods known in the art, for example such as those described in Meziere et al. (1997) J. Immunol. 159, 3230-3237, which is incorporated herein by reference. This approach involves making pseudopeptides containing changes involving the backbone, and not the orientation of side chains. Retro-inverse peptides, which contain NH—CO bonds instead of CO—NH peptide bonds, are much more resistant to proteolysis. Alternatively, the collagen VI or polypeptide of the invention may be a peptidomimetic compound wherein one or more of the amino acid residues are linked by a -γ(CH₂NH)-bond in place of the conventional amide linkage.

In a further alternative, the peptide bond may be dispensed with altogether provided that an appropriate linker moiety which retains the spacing between the carbon atoms of the amino acid residues is used; it may be advantageous for the linker moiety to have substantially the same charge distribution and substantially the same planarity as a peptide bond.

It will be appreciated that at least one of the polypeptides may conveniently be blocked at its N- or C-terminal region so as to help reduce susceptibility to exoproteolytic digestion.

A variety of uncoded or modified amino acids such as ID-amino acids and N-methyl amino acids have also been used to modify mammalian peptides. In addition, a presumed bioactive conformation may be stabilised by a covalent modification, such as cyclisation or by incorporation of lactam or other types of bridges, for example see Veber et al., 1978, Proc. Natl. Acad. Sci. USA 75:2636 and Thursell et al., 1983, Biochem. Biophys. Res. Comm. 111:166, which are incorporated herein by reference.

A common theme among many of the synthetic strategies has been the introduction of some cyclic moiety into a peptide-based framework. The cyclic moiety restricts the conformational space of the peptide structure and this frequently results in an increased specificity of the peptide for a particular biological receptor. An added advantage of this strategy is that the introduction of a cyclic moiety into a peptide may also result in the peptide having a diminished sensitivity to cellular peptidases.

Thus, exemplary polypeptides of the composition of the first aspect comprise terminal cysteine amino acids. Such a polypeptide may exist in a heterodetic cyclic form by disulphide bond formation of the mercaptide groups in the terminal cysteine amino acids or in a homodetic form by amide peptide bond formation between the terminal amino acids. As indicated above, cyclising small peptides through disulphide or amide bonds between the N- and C-terminal region cysteines may circumvent problems of specificity and half-life sometime observed with linear peptides, by decreasing proteolysis and also increasing the rigidity of the structure, which may yield higher specificity compounds. Polypeptides cyclised by disulphide bonds have free amino and carboxy-termini which still may be susceptible to proteolytic degradation, while peptides cyclised by formation of an amide bond between the N-terminal amine and C-terminal carboxyl and hence no longer contain free amino or carboxy termini. Thus, the peptides of the composition of the present invention can be linked either by a C—N linkage or a disulphide linkage.

The present invention is not limited in any way by the method of cyclisation of peptides, but encompasses peptides whose cyclic structure may be achieved by any suitable method of synthesis. Thus, heterodetic linkages may include, but are not limited to formation via disulphide, alkylene or sulphide bridges. Methods of synthesis of cyclic homodetic peptides and cyclic heterodetic peptides, including disulphide, sulphide and alkylene bridges, are disclosed in U.S. Pat. No. 5,643,872, which is incorporated herein by reference. Other examples of cyclisation methods include cyclization through click chemistry, epoxides, aldehyde-amine reactions, as well as and the methods disclosed in U.S. Pat. No. 6,008,058, which is incorporated herein by reference.

A further approach to the synthesis of cyclic stabilised peptidomimetic compounds is ring-closing metathesis (RCM). This method involves steps of synthesising a peptide precursor and contacting it with an RCM catalyst to yield a conformationally restricted peptide. Suitable peptide precursors may contain two or more unsaturated C—C bonds. The method may be carried out using solid-phase-peptide-synthesis techniques. In this embodiment, the precursor, which is anchored to a solid support, is contacted with a RCM catalyst and the product is then cleaved from the solid support to yield a conformationally restricted peptide.

Another approach, disclosed by D. H. Rich in Protease Inhibitors, Barrett and Selveson, eds., Elsevier (1986), which is incorporated herein by reference, has been to design peptide mimics through the application of the transition state analogue concept in enzyme inhibitor design. For example, it is known that the secondary alcohol of staline mimics the tetrahedral transition state of the scissile amide bond of the pepsin substrate.

In summary, terminal modifications are useful, as is well known, to reduce susceptibility by proteinase digestion and therefore to prolong the half-life of the peptides in solutions, particularly in biological fluids where proteases may be present. Polypeptide cyclisation is also a useful modification because of the stable structures formed by cyclisation and in view of the biological activities observed for cyclic peptides.

Thus, in one embodiment at least one of the polypeptides of the composition of the first aspect of the invention is linear. However, in an alternative embodiment, the polypeptide is cyclic.

It will be appreciated by persons skilled in the art that the polypeptides of the composition of the invention may be of various lengths. Typically, however, the polypeptide is between 10 and 200 amino acids in length, for example between 10 and 150, 15 and 100, 15 and 50, 20 and 40, 25 and 35, or 28 and 33 amino acids in length. For example, the polypeptide may be at least 20 amino acids in length. At least one of the polypeptides may be at least 28 amino acids in length. At least one of the polypeptides may be at most 76 amino acids in length, for example at most 36 or 33 amino acids in length.

Thus, in one embodiment of the invention, the polypeptides of the composition of the invention may comprise a specified sequence of any one of SEQ ID NOs: 1 to 23 as part of a longer amino acid sequence. For example, at least one of the polypeptides may comprise any one of SEQ ID NOs: 1 to 23 (or a variant or fragment thereof) as part of an amino acid sequence that is up to 25, 28, 30, 33, 35, 40, 45, 50, 55, 60, 65, 70, 75, 80, 85, 90, 95 or 100 amino acids in length. In a further example, at least one of the polypeptides may comprise any one of SEQ ID NOs: 1 to 23 (or a variant or fragment thereof), wherein the polypeptide is between 10 and 200 amino acids in length, for example between 20 and 200, 28 and 200, 33 and 200, 28 and 150, 33 and 150, 28 and 100, 33 and 100, 28 and 50, 33 and 50, 28 and 40, 33 and 40, or 28 and 33 amino acids in length.

In one embodiment, the collagen VI or at least one of the polypeptides of the composition is or comprises a recombinant polypeptide. Suitable methods for the production of such recombinant polypeptides are well known in the art, such as expression in prokaryotic or eukaryotic hosts cells (for example, see Sambrook & Russell, 2000, Molecular Cloning, A Laboratory Manual, Third Edition, Cold Spring Harbor, New York, the relevant disclosures in which document are hereby incorporated by reference).

Collagen VI or polypeptides of the composition of the invention can also be produced using a commercially available in vitro translation system, such as rabbit reticulocyte lysate or wheatgerm lysate (available from Promega). Preferably, the translation system is rabbit reticulocyte lysate. Conveniently, the translation system may be coupled to a transcription system, such as the TNT transcription-translation system (Promega). This system has the advantage of producing suitable mRNA transcript from an encoding DNA polynucleotide in the same reaction as the translation.

It will be appreciated by persons skilled in the art that collagen VI or polypeptides of the composition of the invention may alternatively be synthesised artificially, for example using well known liquid-phase or solid phase synthesis techniques (such as t-Boc or Fmoc solid-phase peptide synthesis). For example, the polypeptides may be synthesized as described in Solid-Phase Peptide Synthesis (1997) Fields, Abelson & Simon (Eds), Academic Press (ISBN: 0-12-182190-0), which is incorporated herein by reference.

In one embodiment, the composition further comprises polylysine. In some embodiments, the composition does not comprise polylysine. Suitable polylysine components are further defined in PCT/EP2020/068047, the contents of which are incorporated herein by reference.

As used herein, “polylysine” includes any compound that is a polymer of lysine monomer units, preferably joined by peptide bonds. For example, polylysine may include any compound comprising or consisting of 3 or more lysine residues that have been joined together by polymerisation at either the E or a carbon position.

By “polymer” we mean a substance that is made up of multiple monomer units joined together by chemical bonds. Polymers can either form linear chains of molecules or three dimensional networks depending on the type of molecule to be polymerised and the position of polymerisation.

By “polymerisation” we refer to the process used to form a polymer in which multiple monomer units form chemical bonds resulting in the formation of linear polymer chains or a three-dimensional network of molecules. In the case of polymerisation of amino acids such as lysine as described herein, polymerisation involves the formation of peptide bonds between amino acid monomers by reaction of an amino group and a carboxylic acid group. Formation of peptide bonds is a process that is well known in the art.

Polylysine may differ in both the enantiomer of lysine used (i.e. L or D lysine) and the carbon position of polymerisation (i.e. a or E).

Lysine is found as two different enantiomers that differ in the arrangement of R— groups around the chiral centre, termed the L- and D-forms. Other forms of naming enantiomers are used in the art (for example R and S notation, where 5-lysine corresponds to L-lysine and R-lysine corresponds to D-lysine), however L/D notation remains the most commonly used in relation to amino acids. The difference between L- and D-amino acids are well known in the art.

The polylysine of the invention may include polymers comprising or consisting of both L-lysine and D-lysine monomer units, or may comprise or consist of only L-lysine or only D-lysine monomer units in which case the polymers are termed poly-L-lysine (PLL) and poly-D-lysine (PDL), respectively.

In one embodiment the polylysine comprises or consists of poly-L-lysine (PDL) and/or poly-D-lysine (PLL).

Optical isomers such as PLL/PDL have different effects on plane-polarised light (light that travels in a single plane). One isomer will rotate the plane of this plane-polarised light clockwise, and the other will rotate it anticlockwise. This is how the isomers can be distinguished from one another.

In a further embodiment the polylysine comprises or consists of poly-L-lysine, optionally wherein 100% of the monomer units making up the polylysine are L-lysine.

In another embodiment, the polylysine comprises or consists of poly-D-lysine, optionally wherein 100% of the monomer units making up the polylysine are D-lysine.

The interchangeability of PLL and PDL is well known in the art as they both have the same charge-related properties. See, for example, Banker, G. and Goslin, K., Culturing Nerve Cells, MIT Press, Cambridge, p. 65 (1991).

In one embodiment, the polylysine may comprise a mixture of L-lysine and D-lysine monomer units. For example, 50% of the lysine monomers may be L-lysine and 50% may be D-lysine, or alternative ratios of D-lysine:L-lysine may be used, for example: 90:10; 80:20; 70:30; 60:40; 50:50; 40:60; 30:70; 20:80; 10:90.

As the R-group of lysine also contains an amino group (CH₂CH₂CH₂CH₂NH₂), there are two possible polymerization positions, either involving the α carbon or the ε carbon. By “α carbon” we mean the carbon atom in the backbone of the amino acid to which both the amine and carboxylic acid groups are attached. The carbon atoms in the lysine R group are then labelled sequentially (i.e. the first carbon of the R group attached to the α carbon is termed the β carbon, followed by the γ carbon, δ carbon and ε carbon atoms respectively in the carbon chain). Therefore, by “ε carbon” we mean the terminal carbon of the lysine R group to which the amine group is attached.

In one embodiment, the polylysine of the composition of the invention is polymerized at the ε carbon position. In an alternative embodiment, the polylysine is polymerized at the α carbon position.

In one embodiment, 100% of the polymerisation occurs at the ε carbon position. In an alternative embodiment, the polylysine comprises lysine monomer units polymerized at both the α and ε positions within the same molecule, for example 50% of the polymerisation may occur at the α position and 50% of the polymerization may occur at the ε position of the lysine monomer units. Polymerisation at the α or ε positions may also occur in different ratios, for example in the following ratios of α:ε polymerisation positions: 100:0; 90:10; 80:20; 70:30; 60:40; 50:50; 40:60; 30:70; 20:80; 10:90; 0:100.

The polylysine of the composition may be a mixture of different types of polylysine. In certain embodiments, the composition may comprise combinations of any one of the following: ε poly-L-lysine; α poly-L-lysine; ε poly-D-lysine; α poly-D-lysine. The different forms of polylysine may be found in different proportions within the composition, which may be adjusted to achieve optimal binding of the polypeptide to the surface in question. For example, in certain embodiments, the composition may comprise 50% E poly-L-lysine and 50% E poly-D-lysine. The composition may comprise equal or unequal amounts of all four variants of polylysine.

In one embodiment, the polylysine is poly-L-lysine (PLL) and is polymerised at the ε position of the lysine monomers.

In certain embodiments the polylysine may be modified. For example, either the lysine monomers that make up the polylysine may be modified prior to polymerisation or the polylysine itself may be modified after polymerisation.

Polylysine molecules are polymers which can vary in their molecular weight. Commercially available forms of polylysine are often found as compositions comprising polymers of a range of molecular weights rather than as a single molecular weight.

Typically, this range of molecular weights of the polymers is from 30,000 Da to 300,000 Da.

In certain embodiments, the polylysine of the composition of the invention has a molecular weight in the range 30,000 Da to 300,000 Da. In other embodiments, the polylysine has a molecular weight in the range 50,000-250,000 Da; 70,000-200,000 Da; or 100,000-150,000 Da. In one embodiment, the polylysine molecules have a molecular weight in the range 30,000 to 70,000 Da. In one embodiment, the polylysine is poly-L-lysine and the poly-L-lysine molecules have a molecular weight in the range 30,000 to 70,000 Da.

Polylysine can also be categorised in terms of the number of lysine monomer units polymerised together. As the molecular weight of lysine is approximately 146 Da, polymers of polylysine in the molecular weight range 30,000 Da to 300,000 Da are made up of between approximately 200 and 2054 lysine monomer units. Therefore in some embodiments, the polylysine of a composition described herein is made up of between 200 and 2054 lysine monomer units. In other embodiments, the polylysine is poly-L-lysine and the molecules are made up of between 200 and 2054 L-lysine monomer units.

It would be clear to the skilled person that varying the range of molecular weight of the polylysine molecules in the composition would alter the number of lysine residues that make up each polymer molecule. Therefore, in other embodiments, the polylysine molecules of a composition described herein are made up of between: 342-1712 lysine monomers; 479-1369 lysine monomer units; 684-1027 lysine monomer units. In one embodiment, the polylysine molecules of a composition described herein are made up of between 200 and 480 lysine monomer units, corresponding to a molecular weight range of 30,000 to 70,000 Da. In other embodiments, the polylysine is poly-L-lysine and the molecules are made up of between 200 and 480 lysine monomer units.

In a further embodiment, the composition may additionally comprise a scaffold material, such as a biological and/or biodegradable material. The scaffold material may comprise or consist of collagen, for example collagen I. In some embodiments, the collagen further comprises other proteins, polysaccharides and/or peptides. In some embodiments, the scaffold is prepared from bovine collagen I. In some embodiments, the scaffold does not comprise polylysine. In some embodiment the scaffold material comprises bovine native collagen I fibrils.

A composition comprising a scaffold may be construed as a scaffold per se. Accordingly, in a further embodiment, a scaffold comprising the composition may be for a use as described herein. Such a scaffold may be a medical device. The scaffold may be a carrier for effector molecules (i.e. peptides) of the present invention.

It will be appreciated that the compositions described herein may be formulated for use in clinical medicine and/or veterinary medicine.

Thus, a second aspect of the invention provides a pharmaceutical composition comprising a composition according to the first aspect of the invention together with a pharmaceutically acceptable excipient, diluent, carrier, buffer or adjuvant. In a further embodiment, individual peptides as described herein may be formulated as separate pharmaceutical compositions for co-administration, sequentially, subsequently and/or simultaneously to each other.

As used herein, “pharmaceutical composition” means a therapeutically effective formulation for use in the treatment or prevention of disorders and conditions associated with microorganisms and microbial infections.

Additional compounds may also be included in the pharmaceutical compositions, such as other peptides, low molecular weight immunomodulating agents, receptor agonists and antagonists, and antimicrobial agents. Other examples include chelating agents such as EDTA, citrate, EGTA or glutathione.

The pharmaceutical compositions may be prepared in a manner known in the art that is sufficiently storage stable and suitable for administration to humans and animals. The pharmaceutical compositions may be lyophilised, e.g. through freeze drying, spray drying, spray cooling, or through use of particle formation from supercritical particle formation.

By “pharmaceutically acceptable” we mean a non-toxic material that does not decrease the effectiveness of the biological activity of the active ingredients, i.e. the antimicrobial polypeptide(s) of the composition. Such pharmaceutically acceptable buffers, carriers or excipients are well-known in the art (see Remington's Pharmaceutical Sciences, 18th edition, A. R Gennaro, Ed., Mack Publishing Company (1990) and handbook of Pharmaceutical Excipients, 3rd edition, A. Kibbe, Ed., Pharmaceutical Press (2000), which are incorporated herein by reference).

The term “buffer” is intended to mean an aqueous solution containing an acid-base mixture with the purpose of stabilising pH. Examples of buffers are Trizma, Bicine, Tricine, MOPS, MOPSO, MOBS, Tris, Hepes, HEPBS, MES, phosphate, carbonate, acetate, citrate, glycolate, lactate, borate, ACES, ADA, tartrate, AMP, AMPD, AMPSO, BES, CABS, cacodylate, CHES, DIPSO, EPPS, ethanolamine, glycine, HEPPSO, imidazole, imidazolelactic acid, PIPES, SSC, SSPE, POPSO, TAPS, TABS, TAPSO and TES.

The term “diluent” is intended to mean an aqueous or non-aqueous solution with the purpose of diluting the peptide in the pharmaceutical preparation. The diluent may be one or more of saline, water, polyethylene glycol, propylene glycol, ethanol or oils (such as safflower oil, corn oil, peanut oil, cottonseed oil or sesame oil).

The term “adjuvant” is intended to mean any compound added to the formulation to increase the biological effect of the peptide of the composition. The adjuvant may be one or more of colloidal silver, or zinc, copper or silver salts with different anions, for example, but not limited to fluoride, chloride, bromide, iodide, tiocyanate, sulfite, hydroxide, phosphate, carbonate, lactate, glycolate, citrate, borate, tartrate, and acetates of different acyl composition. The adjuvant may also be cationic polymers such as PHMB, cationic cellulose ethers, cationic cellulose esters, deacetylated hyaluronic acid, chitosan, cationic dendrimers, cationic synthetic polymers such as poly(vinyl imidazole), and cationic polypeptides such as polyhistidine, polylysine, polyarginine, and peptides containing these amino acids.

The excipient may be one or more of carbohydrates, polymers, lipids and minerals. Examples of carbohydrates include lactose, sucrose, mannitol, and cyclodextrines, which are added to the composition, e.g., for facilitating lyophilisation. Examples of polymers are starch, cellulose ethers, cellulose, carboxymethylcellulose, hydroxypropylmethyl cellulose, hydroxyethyl cellulose, ethylhydroxyethyl cellulose, ethyl cellulose, methyl cellulose, propyl cellulose, alginates, carageenans, hyaluronic acid and derivatives thereof, polyacrylic acid, polysulphonate, polyethylenglycol/polyethylene oxide, polyethyleneoxide/polypropylene oxide copolymers, polyvinylalcohol/polyvinylacetate of different degree of hydrolysis, poly(lactic acid), poly(glycholic acid) or copolymers thereof with various composition, and polyvinylpyrrolidone, all of different molecular weight, which are added to the composition, e.g. for viscosity control, for achieving bioadhesion, or for protecting the active ingredient (applies to A-C as well) from chemical and proteolytic degradation.

Examples of lipids are fatty acids, phospholipids, mono-, di-, and triglycerides, ceramides, sphingolipids and glycolipids, all of different acyl chain length and saturation, egg lecithin, soy lecithin, hydrogenated egg and soy lecithin, which are added to the composition for reasons similar to those for polymers. Examples of minerals are talc, magnesium oxide, zinc oxide and titanium oxide, which are added to the composition to obtain benefits such as reduction of liquid accumulation or advantageous pigment properties.

The pharmaceutical composition may also contain one or more mono- or di-saccharides such as xylitol, sorbitol, mannitol, lactitiol, isomalt, maltitol or xylosides, and/or monoacylglycerols, such as monolaurin. The characteristics of the carrier are dependent on the route of administration. One route of administration is topical administration. For example, for topical administrations, a preferred carrier is an emulsified cream comprising the active peptide, but other common carriers such as certain petrolatum/mineral-based and vegetable-based ointments can be used, as well as polymer gels, liquid crystalline phases and microemulsions.

It will be appreciated that the pharmaceutical compositions may comprise one or more of the polypeptides of the composition of the first aspect, for example one, two, three, four or more different peptides and/or different fragments, variants or derivatives of said peptides. For example, a composition of the first aspect may comprise a polypeptide according to any one of SEQ ID NO:1 to 23, and a polypeptide fragment, variant and/or derivative of said SEQ ID NO:1 to 23. By using a combination of different peptides, the promotion of wound healing and/or antimicrobial effect may be increased. In some embodiments, the composition does not comprise a polypeptide corresponding to SEQ ID NO: 6.

As discussed above, at least one of the polypeptides may be provided as a salt, for example an acid adduct with inorganic acids, such as hydrochloric acid, sulfuric acid, nitric acid, hydrobromic acid, phosphoric acid, perchloric acid, thiocyanic acid, boric acid etc. or with organic acid such as formic acid, acetic acid, haloacetic acid, propionic acid, glycolic acid, citric acid, tartaric acid, succinic acid, gluconic acid, lactic acid, malonic acid, fumaric acid, anthranilic acid, benzoic acid, cinnamic acid, p-toluenesulfonic acid, naphthalenesulfonic acid, sulfanilic acid etc. Inorganic salts such as monovalent sodium, potassium or divalent zinc, magnesium, copper calcium, all with a corresponding anion, may be added to improve the biological activity of the antimicrobial composition.

The pharmaceutical compositions of the invention may also be in the form of a liposome, in which the polypeptide is combined, in addition to other pharmaceutically acceptable carriers, with amphipathic agents such as lipids, which exist in aggregated forms as micelles, insoluble monolayers and liquid crystals. Suitable lipids for liposomal formulation include, without limitation, monoglycerides, diglycerides, sulfatides, lysolecithin, phospholipids, saponin, bile acids, and the like. Suitable lipids also include the lipids above modified by poly(ethylene glycol) in the polar headgroup for prolonging bloodstream circulation time. Preparation of such liposomal formulations is can be found in for example U.S. Pat. No. 4,235,871, which is incorporated herein by reference.

The pharmaceutical compositions of the invention may also be in the form of biodegradable microspheres. Aliphatic polyesters, such as poly(lactic acid) (PLA), poly(glycolic acid) (PGA), copolymers of PLA and PGA (PLGA) or poly(caprolactone) (PCL), and polyanhydrides have been widely used as biodegradable polymers in the production of microshperes. Preparations of such microspheres can be found in U.S. Pat. No. 5,851,451 and in EP 213 303, which are incorporated herein by reference.

The pharmaceutical compositions of the invention may also be formulated with micellar systems formed by surfactants and block copolymers, preferably those containing poly(ethylene oxide) moieties for prolonging bloodstream circulation time.

The pharmaceutical compositions of the invention may also be in the form of polymer gels, where polymers such as starch, cellulose ethers, cellulose, carboxymethylcellulose, hydroxypropylmethyl cellulose, hydroxyethyl cellulose, ethylhydroxyethyl cellulose, ethyl cellulose, methyl cellulose, propyl cellulose, alginates, chitosan, carrageenans, hyaluronic acid and derivatives thereof, polyacrylic acid, polyvinyl imidazole, polysulphonate, polyethyleneglycol/polyethylene oxide, polyethylene-oxide/polypropylene oxide copolymers, polyvinylalcohol/polyvinylacetate of different degree of hydrolysis, and polyvinylpyrrolidone are used for thickening of the solution containing the peptide. The polymers may also comprise gelatin or collagen.

Alternatively, the collagen VI or polypeptide of the composition may be dissolved in saline, water, polyethylene glycol, propylene glycol, ethanol or oils (such as safflower oil, corn oil, peanut oil, cottonseed oil or sesame oil), tragacanth gum, and/or various buffers.

The pharmaceutical composition may also include ions and a defined pH for potentiation of action of anti-microbial polypeptides and/or for potentiation of action of wound healing polypeptides.

The above compositions of the invention may be subjected to conventional pharmaceutical operations such as sterilisation and/or may contain conventional adjuvants such as preservatives, stabilisers, wetting agents, emulsifiers, buffers, fillers, etc., e.g., as disclosed elsewhere herein.

It will be appreciated by persons skilled in the art that the pharmaceutical compositions of the invention may be administered locally or systemically. Routes of administration include topical (e.g. ophthalmic), ocular, nasal, pulmonary, buccal, parenteral (intravenous, subcutaneous, and intramuscular), oral, vaginal and rectal. Also, administration from implants is possible. Suitable preparation forms are, for example granules, powders, tablets, coated tablets, (micro) capsules, suppositories, syrups, emulsions, microemulsions, defined as optically isotropic thermodynamically stable systems consisting of water, oil and surfactant, liquid crystalline phases, defined as systems characterised by long-range order but short-range disorder (examples include lamellar, hexagonal and cubic phases, either water- or oil continuous), or their dispersed counterparts, gels, ointments, dispersions, suspensions, creams, aerosols, droplets or injectable solution in ampoule form and also preparations with protracted release of active compounds, in whose preparation excipients, diluents, adjuvants or carriers are customarily used as described above. The pharmaceutical composition may also be provided in bandages, plasters or in sutures or the like.

In a particular embodiment, the pharmaceutical composition is suitable for oral administration, parenteral administration or topical administration. For example, the pharmaceutical composition may be suitable for topical administration (e.g. ophthalmic administration, in the form of a spray, lotion, paste or drops etc.).

The pharmaceutical compositions will be administered to a patient in a pharmaceutically effective dose. By “pharmaceutically effective dose” is meant a dose that is sufficient to produce the desired effects in relation to the condition for which it is administered. The exact dose is dependent on the, activity of the compound, manner of administration, nature and severity of the disorder, age and body weight of the patient different doses may be needed. The administration of the dose can be carried out both by single administration in the form of an individual dose unit or else several smaller dose units and also by multiple administrations of subdivided doses at specific intervals.

The pharmaceutical compositions of the invention may be administered alone or in combination with other therapeutic agents, such as additional antibiotic, anti-inflammatory, immunosuppressive, vasoactive and/or antiseptic agents (such as anti-bacterial agents, anti-fungicides, anti-viral agents, and anti-parasitic agents).

Examples of suitable additional antibiotic agents include penicillins, cephalosporins, carbacephems, cephamycins, carbapenems, monobactams, aminoglycosides, glycopeptides, quinolones, tetracyclines, macrolides, and fluoroquinolones. Antiseptic agents include iodine, silver, copper, chlorhexidine, polyhexanide and other biguanides, chitosan, acetic acid, and hydrogen peroxide. Likewise, the pharmaceutical compositions may also contain anti-inflammatory drugs, such as steroids and macrolactam derivatives.

Such additional therapeutic agents may be incorporated as part of the same pharmaceutical composition or may be administered separately. Additional therapeutic agents can be administered simultaneously, sequentially and/or separately, either before, after or during administration of a pharmaceutical composition of the second aspect.

It will be appreciated by persons skilled in the art that the compositions of the invention, or pharmaceutical compositions thereof, may be applied to medical devices and other products the implantation into or application of which to the human or animal body is associated with the risk of infection by a microbial agent; and/or the compositions of the invention, or pharmaceutical compositions thereof, may be applied to medical devices and other products the implantation into or application of which to the human or animal body is associated with a need to promote wound healing.

Thus, a third aspect of the invention provides a medical device, implant, wound care product, or material for use in the same, which is coated, impregnated, admixed or otherwise associated with a composition according to the first aspect of the invention or a pharmaceutical composition according to the second aspect of the invention.

Such a medical device, implant, wound care product, or material for use in the same may come into contact with the human body or component thereof (e.g. blood).

In one embodiment, the medical device, implant, wound care product, or material for use in the same is for use in by-pass surgery, extracorporeal circulation, wound care and/or dialysis.

The composition may be coated, painted, sprayed or otherwise applied to or admixed with a suture, prosthesis, implant, wound dressing, catheter, lens, skin graft, skin substitute, fibrin glue or bandage, etc. In so doing, the composition may impart improved antimicrobial and/or wound healing properties to the device or material.

By “implant”, we include:
(a) A catheter (for example, for intravascular or urinary tract use);
(b) A stent (for example, a coronary stent);
(c) A shunt (for example, a cerebrospinal shunt);
(d) An intubating or tracheotomy tube;
(e) An ophthalmic device (for example, contact lenses, scleral buckles and intraocular lenses);
(f) A joint prosthesis (i.e. arthroplasty and implantation of other orthopaedic devices);
(g) An artificial heart valve;
(h) A breast implant;
(i) An implantable drug delivery device (for example, active pumps and passive solid implants).

In some preferred embodiments, the implant is a prosthesis or other orthopaedic device. In some preferred embodiments, the prosthesis is a knee joint prosthesis. In other preferred embodiments, the prosthesis is a hip joint prosthesis. Further examples of such implants are well known in the art. In further preferred embodiments the implant is a prosthesis or other orthopaedic device comprising or consisting of titanium or a titanium alloy, and/or one or more ceramic composite. In one embodiment, the prosthesis or other orthopaedic device comprises or consists of titanium or a titanium alloy, and/or one or more ceramic composite, and is coated with a composition or pharmaceutical composition as defined herein.

In other embodiments, the implant is selected from: bone replacement devices, bone fixation devices, bone plates, stems of artificial hip joints, artificial organs, artificial intervertebral discs, spinal rods, maxillofacial plates, stent grafts, percutaneous devices and pacemakers. In one embodiment these implants comprise or consist of titanium or a titanium alloy, and/or one or more ceramic composite. In further embodiments, the implant comprises or consists of titanium or a titanium alloy, and/or one or more ceramic composite, and is coated with a composition or pharmaceutical composition as defined herein.

In one embodiment, the device or material is coated with the composition or pharmaceutical composition of the invention (or at least one of the polypeptide components thereof). By ‘coated’ we mean that the composition or pharmaceutical composition is applied to the surface of the device or material. Thus, the device or material may be painted or sprayed with a solution comprising a composition or pharmaceutical composition of the invention (or at least one of the polypeptides thereof). Alternatively, the device or material may be dipped in a reservoir of a solution comprising a composition or pharmaceutical composition of the invention.

In one embodiment, the medical device, implant, wound care product, or material for use in the same is coated with a composition or pharmaceutical composition as defined herein.

In an alternative embodiment, the device or material is impregnated with a pharmaceutical composition of the invention (or collagen VI or at least one of the polypeptides thereof). By ‘impregnated’ we mean that the pharmaceutical composition is incorporated or otherwise mixed with the device or material such that it is distributed throughout.

For example, the device or material may be incubated overnight at 4° C. in a solution comprising a composition or pharmaceutical composition of the invention. Alternatively, a composition or pharmaceutical composition of the invention may be immobilised on the device or material surface by evaporation or by incubation at room temperature. As a further alternative, a composition or pharmaceutical composition (or, indeed, a polypeptide) of the invention may be immobilised on the device or material surface while a further composition or pharmaceutical composition is not. When compositions, pharmaceutical compositions and/or polypeptides are administered separately (e.g. one being immobilised and the other not), their administration may be in such a way to jointly expose each (or all, if more than two) to the host biomaterial interface.

In a further alternative embodiment, a polypeptide of the composition of the invention is covalently linked to the device or material, e.g. at the external surface of the device or material. Thus, a covalent bond is formed between an appropriate functional group on the polypeptide and a functional group on the device or material. For example, methods for covalent bonding of polypeptides to polymer supports include covalent linking via a diazonium intermediate, by formation of peptide links, by alkylation of phenolic, amine and sulphydryl groups on the binding protein, by using a poly functional intermediate e.g. glutaraldehyde, and other miscellaneous methods e.g. using silylated glass or quartz where the reaction of di- and trialkoxysilanes permits derivatisation of the glass surface with many different functional groups. For details, see Enzyme immobilisation by Griffin, M., Hammonds, E. J. and Leach, C. K. (1993) In Technological Applications of Biocatalysts (BIOTOL SERIES), pp. 75-118, Butterworth-Heinemann, incorporated herein by reference. See also the review article entitled ‘Biomaterials in Tissue Engineering’ by Hubbell, J. A. (1995) Science 13:565-576, which is incorporated herein by reference.

In one embodiment, the medical device, implant, wound care product, or material comprises or consists of a polymer. Suitable polymers may be selected from the group consisting of polyesters (e.g. polylactic acid, polyglycolic acid or poly lactic acid-glycolic acid copolymers of various composition), polyorthoesters, polyacetals, polyureas, polycarbonates, polyurethanes, polyamides) and polysaccharide materials (e.g. cross-linked alginates, hyaluronic acid, carrageenans, gelatins, starch, cellulose derivatives).

Alternatively, or in addition, the medical device, implant, wound care product, or material may comprise or consists of metals (e.g. titanium, stainless steel, gold, titanium), metal oxides (silicon oxide, titanium oxide) and/or ceramics (apatite, hydroxyapatite) or ceramic composites.

In one embodiment, the medical device, implant, wound care product or material for use in the same comprises or consists of titanium. In one embodiment, the medical device, implant, wound care product or material for use in the same comprises or consists of one or more ceramic composites.

By “comprises or consists of titanium” we include materials made solely of pure titanium and also materials that comprise titanium in combination with other elements. For example, by “comprises or consists of titanium” we also include materials that are or comprise titanium alloys (for example alloys of titanium with nickel, vanadium and/or aluminium). We also include medical devices, implants, wound care products and materials for use in the same that have at least one component comprising or consisting of titanium and/or one or more titanium alloys. We also include medical devices, implants, wound care products and materials for use in the same that have a coating comprising or consisting of titanium, for example a coating comprising or consisting of titanium or titanium nitride. It will be clear therefore to the skilled person that mention herein of titanium is also meant to include reference to titanium alloy.

Thus, in one embodiment the titanium is commercially pure titanium (CP Ti). Alternatively, in specific embodiments the titanium is an alloy, for example a Ti₆Al₄V alloy or a nickel-titanium alloy (Nitinol).

By “comprises or consists of one or more ceramic composites” we include materials made solely of a ceramic composite and also materials that comprise a ceramic composite in combination with other elements. We also include medical devices, implants, wound care products and materials for use in the same that have at least one component comprising or consisting of one or more ceramic composites. We also include medical devices, implants, wound care products and materials for use in the same that have a coating comprising or consisting of one or more ceramic composites.

In one embodiment the ceramic composite may be selected from the group consisting of calcium phosphates, hydroxyapatite, calcium sulfate dihydrate, zirconia, aluminium-oxide, Cordierite, Forsterite, silicon nitride, Pyrostat, Steatite and Superpyrostat.

Such materials may be in the form of macroscopic solids/monoliths, as chemically or physicochemically cross-linked gels, as porous materials, or as particles.

Medical devices, implants, wound care products, and materials of the invention may be made using methods well known in the art.

In certain embodiments, the composition of the first aspect of the invention is coated onto a biological scaffold, such as a collagen scaffold, for example a collagen I scaffold. In one embodiment the collagen scaffold may be used directly as a medical device, implant or wound care product or material for use in the same. In other embodiments, the collagen scaffold is a component of a medical device, implant, wound care product or material for use in the same. For example, the collagen scaffold may be coated onto the surface of such a medical device, implant, wound care product or material for use in the same.

In certain embodiments, the scaffold is a collagen scaffold, such as a collagen I scaffold. In certain embodiments, wherein the composition further comprises polylysine, the polylysine forms an intermediate layer between the scaffold and the collagen VI polypeptide.

In certain embodiments the collagen scaffold may be a bovine fibrillar collagen scaffold, comprising collagen I and/or III and/or V and/or VI as the major constituents. For example, 80-85% of the scaffold may be type I collagen fibres and/or 8-11% may be type III collagen fibres and the remainder may be collagen V and/or VI. Preferably, the scaffold is a freeze-dried bovine collagen I/III/V scaffold.

A collagen I/III/V scaffold mixed with the collagen VI peptides of the composition of the first aspect of the invention (such as the product referred to as WOUNDCOM in the examples) offers the wound an interim barrier function and establishes a contained moist wound microenvironment. This microenvironment promotes re-epithelialisation and re-vascularisation and therefore accelerated wound healing [48].

The term “scaffold” and the terms “carrier” or “WOUNDCOM carrier” are used interchangeably herein. Accordingly, all definitions of scaffold also apply to carriers or

WOUNDCOM carriers. Thus in one embodiment, the carrier or WOUNDCOM carrier is a collagen protein, such as a collagen I protein. In certain embodiments the carrier may be a fibrillar collagen carrier, such as a bovine fibrillar collagen carrier, comprising collagen I and/or III and/or V and/or VI as the major constituents. For example, 80-85% of the scaffold may be type I collagen fibres and/or 8-11% may be type III collagen fibres and the remainder may be collagen V and/or VI. Preferably, the carrier is a freeze-dried bovine collagen I/III/V carrier.

The WOUNDCOM product is the combination of a WOUNDCOM carrier and WOUNDCOM effectors—such as a collagen I scaffold impregnated with bioactive peptides (such as GVR28 and SFV33). Thus in one embodiment WOUNDCOM is the collagen I wound matrix, impregnated with a combination of the GVR28 and SFV33 peptides.

Additionally, the highly aligned collagen fibres in collagen based wound care devices wherein a collagen scaffold is present (such as WOUNDCOM) exhibit appropriate porous structures with parallel collagen fibre bundles that mimic the natural structure of native dermis. Hence, a central role of collagen scaffold based wound care devices in the wound bed is to direct and guide cell influx and migration into the wound by providing three-dimensional molecular guiding ridges by native collagen fibrils. Thus, collagen scaffold based wound care devices (such as WOUNDCOM) promote structured wound healing, resulting in new tissue growth by organizing and aligning the deposition of newly formed collagen fibres and other tissue components in the wound bed [43].

Furthermore, the collagen VI-peptides applied in the composition of the first aspect of the invention enhance the body's own wound healing effect. This is achieved by their natural antimicrobial properties, acting on pathogens by physical membrane destabilization, causing cytoplasmic exudation, cell lysis and thereby inhibition of pathogen growth and biofilm formation [49]. The peptides also further accelerate the wound healing process by providing additional structural and functional elements for efficient recruitment, survival and proliferation of skin cells and immune cells beneficial for the wound healing process.

In other embodiments, the composition of the first aspect of the invention is coated directly onto the surface of a medical device, implant, wound care product or material for use in the same, wherein the medical device, implant, wound care product or material for use in the same comprises or consists of titanium or a titanium alloy. In some embodiments the coated titanium surface may be used directly as a medical device, implant or wound care product or material for use in the same. In other embodiments, the coated titanium surface is a component of a medical device, implant, wound care product or material for use in the same.

In certain embodiments, wherein the composition further comprises polylysine, the polylysine forms an intermediate layer between the titanium surface and the collagen VI polypeptide.

In some embodiments the coated titanium surface may be used directly as an implant comprising or consisting of titanium or a titanium alloy, for example as a joint prosthesis or other orthopaedic device, for example as knee joint prostheses or hip joint prostheses. In further embodiments, the coated titanium surface may be used directly as an implant comprising or consisting of titanium or a titanium alloy, for example as bone replacement devices, bone fixation devices, bone plates, stems of artificial hip joints, artificial organs, artificial intervertebral discs, spinal rods, maxillofacial plates, stent grafts, percutaneous devices and pacemakers.

By “titanium surface” we include all medical devices, implants, wound care products or materials for use in the same with surfaces that comprise or consist of titanium or a titanium alloy.

In other embodiments, the composition of the first aspect of the invention is coated directly onto the surface of a medical device, implant, wound care product or material for use in the same, wherein the medical device, implant, wound care product or material for use in the same comprises or consists of one or more ceramic composites. In some embodiments the coated ceramic composite surface may be used directly as a medical device, implant or wound care product or material for use in the same. In other embodiments, the coated ceramic composite surface is a component of a medical device, implant, wound care product or material for use in the same.

In certain embodiments, wherein the composition further comprises polylysine, the polylysine forms an intermediate layer between a ceramic composite surface and the collagen VI polypeptide.

In some embodiments the coated ceramic composite surface may be used directly as an implant comprising or consisting of a ceramic composite, for example as a joint prosthesis or other orthopaedic device, for example as knee joint prostheses or hip joint prostheses. In further embodiments, the coated ceramic composite surface may be used directly as an implant comprising or consisting of a ceramic composite, for example as bone replacement devices, bone fixation devices, bone plates, stems of artificial hip joints, artificial organs, artificial intervertebral discs, spinal rods, maxillofacial plates, stent grafts, percutaneous devices and pacemakers.

By “ceramic composite surface” we include all medical devices, implants, wound care products or materials for use in the same with surfaces that comprise or consist of one or more ceramic composites.

It will be appreciated that any of the medical devices, implants, wound care products, and materials of the invention may be used for any of the medical uses disclosed herein.

In one embodiment the medical device, implant, wound care product or material for use in the same is coated with a composition as defined in the first aspect, such that the composition or pharmaceutical composition is applied to the surface of said material, resulting in the binding of the polypeptides of the composition to the surface of the material.

The skilled person would be aware of techniques available in the art for determining the level of binding of a polypeptide component of a composition to the surface of a material, such as radioactive labelling of peptides. An example of this is labelling with a radioactive isotope of iodine (e.g. iodine 131) by labelling lysine or tyrosine residues, and measuring the level of radioactivity produced by a coated material, which is proportional to the level of binding achieved. The coating efficiencies of the different biomolecules can be assessed by determining the ratio between bound and free 131-iodine radioactivity associated with the material by determining radioactivity as cpm values in a γ-counter, i.e. at a measurement of 100% binding an undetectable amount of radioactive iodine 131 is not bound to the surface of the material.

Other techniques for measuring percentage binding of a peptide or protein to a surface include but are not limited to: spectroscopic assays; radioactivity based binding assays; ellipsometry; single-oscillation quartz crystal thin-film thickness monitoring; fluorescence based binding assays; surface plasmon resonance (SPR) and atomic force microscopy (AFM).

In one embodiment the medical device, wound care product, or material for use in the same may become soft or mouldable when moist. The moisture may be from the wound itself and/or the moisture may be from an external source (e.g. such as saline applied to the medical device, wound care product or material for use in the same). In one embodiment the medical device, wound care product, or material for use in the same may be fixed in place using a secondary dressing and/or compression garment.

A fourth aspect of the invention provides a method of preparing a medical device, implant, wound care product or material for use in the same, comprising the step of coating, impregnating, admixing or otherwise associating the medical device, implant, wound care product, or material for use in the same with the composition or pharmaceutical composition described in the first or second aspects.

The skilled person would understand that the general methods of coating, impregnating and admixing as described above could also be applied to methods of preparing medical devices, implants, wound care products or materials for use in the same according to this aspect of the invention.

A fifth aspect of the invention provides a method of preparing a medical device, implant, wound care product or material for use in the same comprising the following steps:
(i) preparing a composition comprising two or more collagen type VI polypeptides, as defined in the first aspect; and
(ii) coating, impregnating, admixing or otherwise associating the medical device, implant, wound care product, or material for use in the same with the composition prepared in step (i).

Thus, medical devices, implants, wound care products and materials for use herein may be coated, impregnated, admixed or otherwise associated with a composition of the first aspect of the invention by utilising a single composition comprising both the polypeptides of the composition of the first aspect. The medical device, implant, wound care product or material for use in the same may preferably be coated with such a composition.

In one embodiment, the medical device, implant, wound care product or material for use in the same comprises or consists of a titanium surface.

In one embodiment, the medical device, implant, wound care product or material for use in the same comprises or consists of a ceramic composite surface.

A sixth aspect of the invention provides a method of preparing a medical device, implant, wound care product or material for use in the same comprising the following steps:
(i) coating, impregnating, admixing or otherwise associating the medical device, implant, wound care product, or material for use in the same with a first collagen type VI polypeptide; and
(ii) coating, impregnating, admixing or otherwise associating the medical device, implant, wound care product, or material for use in the same produced in step (i) with a second collagen type VI polypeptide;
   optionally comprising the step of coating, impregnating, admixing or otherwise associating the medical device, implant, wound care product, or material for use in the same with polylysine as defined herein.

In one embodiment, the first and second polypeptides are coated simultaneously (for example, as a fusion or as a mixture). Alternatively, the polypeptides may be coated sequentially in any order.

An alteration to the sixth aspect of the invention provides a method of preparing a medical device, implant, wound care product or material for use in the same comprising the following steps:
(i) coating, impregnating, admixing or otherwise associating the medical device, implant, wound care product, or material for use in the same with polylysine as defined herein;
(ii) coating, impregnating, admixing or otherwise associating the medical device, implant, wound care product, or material for use in the same with a first collagen type VI polypeptide; and
(iii) coating, impregnating, admixing or otherwise associating the medical device, implant, wound care product, or material for use in the same with a second collagen type VI polypeptide;
   optionally wherein the first and second polypeptides are coated simultaneously (for example, as a fusion) or sequentially.

Thus, medical devices, implants, wound care products and materials for use herein may be prepared by first coating, impregnating, admixing or otherwise associating the material with a first collagen type VI polypeptide as defined in the first aspect, followed by an additional step in which a second collagen type VI polypeptide as defined in the first aspect is applied subsequently. Alternatively, the polypeptides may be applied at the same time in a single step of coating.

In certain embodiments, medical devices, implants, wound care products and materials for use herein may be prepared by first coating, impregnating, admixing or otherwise associating the material with polylysine, before applying the collagen type VI polypeptides subsequently.

In certain embodiments, the polylysine is coated onto a scaffold, such as a biological scaffold (e.g. a collagen such as collagen I) prior to coating, impregnating, admixing or otherwise associating the material with at least one of the polypeptides as defined in the first aspect, or vice versa. The scaffold may be present on a titanium surface or a ceramic composite surface.

In other embodiments, the polylysine is not coated onto a scaffold prior to coating, impregnating, admixing or otherwise associating the material with a collagen VI or polypeptide as defined in the first aspect. For example, in some embodiments, the polylysine is coated directly onto a surface, for example a surface comprising or consisting of titanium or a titanium alloy or a surface comprising or consisting of one or more ceramic composites, prior to coating, impregnating, admixing or otherwise associating the material with a collagen VI or polypeptide as defined in the first aspect.

In other embodiments, the polylysine can be mixed with a collagen VI or polypeptide as defined in the first aspect prior to coating, impregnating, admixing or otherwise associating the mixture with a collagen scaffold or other surface e.g. a surface comprising or consisting of titanium or a titanium alloy or a surface comprising or consisting of one or more ceramic composites.

In some embodiments, the medical device, implant, wound care product, or material for use in the same comprises or consists of a titanium surface. In some embodiments, the medical device, implant, wound care product, or material for use in the same comprises or consists of one or more ceramic composite surfaces.

In certain embodiments, the method of preparing a medical device, implant, wound care product or material for use in the same, comprises one or more of the following steps:
(i) incubating a scaffold, e.g. a collagen scaffold, with a solution of polylysine;
(ii) washing the scaffold (e.g. in distilled water);
(iii) drying the scaffold (e.g. by air drying);
(iv) coating the scaffold with at least one collagen VI polypeptide as defined in the first aspect of the invention (e.g. by plunging the scaffold into a solution containing the collagen VI polypeptide(s));
(v) incubating the scaffold in the collagen VI solution (e.g. overnight)
(vi) drying the scaffold (e.g. by air drying).

In certain embodiments of the above method, the collagen scaffold is a collagen I scaffold. In one embodiment, the collagen scaffold is disc shaped or a membrane, e.g. consisting of freeze-dried collagen I.

In certain embodiments of the above method, the polylysine of step (i) is poly-L-lysine. In some embodiments, the concentration of the polylysine solution is approximately 0.2 mg/ml. In one embodiment, the polylysine solution of step (i) is an approximately 0.2 mg/ml solution of poly-L-lysine. In some embodiments, the collagen scaffolds are incubated in the solution of polylysine at approximately 60° C. In some embodiment, the collagen scaffolds are incubated in the solution of polylysine for approximately two hours.

In certain embodiments of the above method, the concentration of collagen VI peptide in the collagen VI peptide solution of step (iv) is approximately 150 mM. In some embodiments, the concentration of collagen VI peptides in the collagen VI solution of step (iv) is approximately 2-3 mM. In some preferred embodiments, the concentration of collagen VI peptides in the collagen VI solution of step (iv) is approximately 2-3 μM. In some embodiments, the concentration of collagen VI peptides in the collagen VI solution of step (iv) is 3 μM, and optionally consists of 1.5 μM GVR28 (SEQ ID NO: 1) and 1.5 μM SFV33 (SEQ ID NO: 5).

In certain embodiments, the collagen scaffolds are incubated in the collagen VI solution overnight, i.e. for approximately 16 hours. In some embodiments, the collagen scaffolds are incubated with the collagen VI solution at approximately 4° C.

In other embodiments of step (i) above the polylysine in step (i) is coated onto the surface of the medical device, implant, wound care product, or material for use in the same, prior to coating, impregnating, admixing or otherwise associating the material with a collagen VI or polypeptide as defined in the first aspect. In some embodiments, the medical device, implant, wound care product, or material for use in the same comprises or consists of a titanium surface. In some embodiments, the medical device, implant, wound care product, or material for use in the same comprises or consists of a ceramic composite surface.

In certain embodiments, the method of preparing a medical device, implant, wound care product or material for use in the same, comprises one or more of the following steps:
(i) incubating a medical device, implant, wound care product or material for use in the same, e.g. a titanium surface or alternatively a ceramic composite surface, with a solution of polylysine;
(ii) washing the surface (e.g. in distilled water);
(iii) drying the surface (e.g. by air drying);
(iv) coating the surface with collagen VI or a collagen VI polypeptide as defined in the first aspect of the invention (e.g. by plunging the surface into a solution containing the collagen VI or collagen VI polypeptide);
(v) incubating the surface in the collagen VI solution (e.g. overnight)
(vi) drying the surface (e.g. by air drying).

In certain embodiments of the above method, the polylysine of step (i) is poly-L-lysine. In some embodiments, the concentration of the polylysine solution is approximately 0.2 mg/ml. In one embodiment, the polylysine solution of step (i) is an approximately 0.2 mg/ml solution of poly-L-lysine. In some embodiments, the surface (e.g. a ceramic composite surface and/or titanium surface) is incubated in the solution of polylysine at approximately 60° C. In some embodiments, the surfaces (e.g. the ceramic composite surfaces and/or titanium surfaces) are incubated in the solution of polylysine for approximately two hours.

In certain embodiments of the above method, the concentration of collagen VI in the collagen VI solution of step (iv) is approximately 150 mM. In some embodiments, the concentration of collagen VI peptides in the collagen VI solution of step (iv) is approximately 2-3 mM. In some preferred embodiments, the concentration of collagen VI peptides in the collagen VI solution of step (iv) is approximately 2-3 μM.

In certain embodiments, the method of preparing a medical device, implant, wound care product or material for use in the same, comprises one or more of the following steps:
(i) mixing a scaffold solution (e.g. a collagen scaffold) with at least one collagen VI polypeptide as defined in the first aspect of the invention (e.g. adding a solution containing the collagen VI polypeptide(s) to a solution of the scaffold); and
(ii) drying the scaffold/polypeptide(s) mixture (e.g. by air drying or freeze drying).

A seventh aspect of the invention provides a kit comprising:
(i) a composition according to any the first aspect of the invention and/or a pharmaceutical composition according to the second aspect of the invention or a medical device, implant, wound care product, or material for use in the same according to a third aspect of the invention, and
(ii) instructions for use.

The kit optionally comprises two or more polypeptides as described in the first aspect of the invention in admixture or as separate components requiring mixture prior to use or upon use. For example, each component may be lyophilised separately or in combination with any one or more further polypeptides, and optionally include in the kit a suitable buffer or buffers for reconstituting and/or mixing polypeptides prior to use.

An eighth aspect of the invention provides a kit comprising:
(i) a collagen type VI polypeptide having the primary function of being capable of promoting wound healing according to the first aspect of the invention;
(ii) a collagen type VI polypeptide having the primary function of being capable of exerting an antimicrobial effect according to the first aspect of the invention; and
(iii) instructions for use;
   optionally wherein the kit further comprises polylysine as described herein.

In certain embodiments of the kits of the seventh and eighth aspects, the kit additionally comprises a scaffold material, such as a biological and/or biodegradable scaffold. The scaffold may comprise or consist of collagen, e.g. collagen I.

In other embodiments of the kits of the seventh and eighth aspects, the kit additionally comprises a material comprising or consisting of titanium or a titanium alloy, for example a medical device, implant, wound care produced or material for use in the same comprising or consisting of titanium.

In other embodiments of the kits of the seventh and eighth aspects, the kit additionally comprises a material comprising or consisting of one or more ceramic composites, for example a medical device, implant, wound care produced or material for use in the same comprising or consisting of a ceramic composite.

A ninth aspect of the invention provides a composition according to the first aspect of the invention, or a pharmaceutical composition according to the second aspect of the invention for use in medicine.

A tenth aspect of the invention provides a composition according to the first aspect of the invention, or a pharmaceutical composition according to the second aspect of the invention for use in the curative and/or prophylactic treatment of microbial infections.

The term ‘prophylactic’ is used to encompass the use of a composition or formulation described herein which either prevents or reduces the likelihood of a condition or disease state in a patient or subject.

By “microbial infections” we include infections caused by microorganisms as described above. For example, in one embodiment the microbial infection to be treated is a bacterial infection. The microbial infection to be treated may be an acute or a systemic infection. In one embodiment, the microbial infection is resistant to one or more conventional antibiotic agents (as discussed above).

In one embodiment, the microbial infection to be treated is caused by a microorganism selected from the group consisting of: Pseudomonas aeruginosa, Staphylococcus aureus, Escherichia coli and Streptococcus pyogenes.

In a further embodiment, the microbial infection is caused by a microorganism selected from the group consisting of: multidrug-resistant Staphylococcus aureus (MRSA) (methicillin-resistant Staphylococcus aureus) and multidrug-resistant Pseudomonas aeruginosa (M RPA).

It will be appreciated by persons skilled in the art that the compositions of the invention may be co-administered in combination with one or more known or conventional agents for the treatment of the particular disease or condition. By ‘co-administer’ it is meant that the present compositions are administered to a patient such that the components of the composition as well as the co-administered compound may be found in the patient's body (e.g. in the bloodstream) at the same time, regardless of when the compounds are actually administered, including simultaneously, sequentially and/or subsequently.

Therefore, in one embodiment the composition or pharmaceutical composition is for use in combination with one or more additional antimicrobial agents, such as the conventional antibiotics described above. Alternatively, or in addition, the additional antimicrobial agents may be an antimicrobial polypeptide or protein, such as LL-37 and collagen type VI protein, or for example selected from group consisting of defensins, gramicidin 5, magainin, cecropin, histatin, hyphancin, cinnamycin, burforin 1, parasin 1 and protamines, and fragments, variants and fusion thereof which retain, at least in part, the antimicrobial activity of the parent protein.

An eleventh aspect of the invention provides a composition according to the first aspect of the invention or a pharmaceutical composition according to the second aspect of the invention for use in wound care (i.e. for use in the promotion of wound healing).

By “wound care” we include the treatment of wounds, promoting wound closure (i.e. healing), preventing and/or treating wound infection and/or ulcers, wherein the wound may be extracorporeal or intracorporeal. Use in wound care therefore includes compositions comprising polypeptides which are able to aid (for example, accelerate or improve the efficiency of) the wound healing process, reduce abnormal scar formation, and/or to prevent infection of the wound. For example, the collagen VI or polypeptide of the composition may be used in a wound care product, such as a cream, gel, ointment, dressing or plaster, which is capable of enhancing epithelial regeneration and/or healing of wound epithelia and/or wound stroma. In one embodiment, the at least one of the polypeptides is capable of enhancing the proliferation of epithelial and/or stromal cells through a non-lytic mechanism.

It will be appreciated that the collagen VI and polypeptides having wound healing properties may have a primary or ancillary role in the function of the wound care products of the invention.

In one embodiment, the collagen VI or at least one of the polypeptides or pharmaceutical composition is administered in combination with an additional antimicrobial agent, as described above.

A twelfth aspect of the invention provides the use of a composition according to the first aspect of the invention, or a pharmaceutical composition according to the second aspect of the invention in the manufacture of a medicament for the treatment of microbial infections, as described above.

A thirteenth aspect of the invention provides the use of a composition according to the first aspect of the invention, or a pharmaceutical composition according to the second aspect of the invention in the manufacture of a medicament for the treatment of wounds, as described above.

A fourteenth aspect of the invention provides a method of treating an individual with a microbial infection, the method comprising the step of administering to an individual in need thereof an effective amount of a composition according to the first aspect of the invention, or a pharmaceutical composition according to the second aspect of the invention.

A fifteenth aspect of the invention provides a method of treating a wound in an individual, the method comprising the step of administering to an individual in need thereof an effective amount of a composition according to the first aspect of the invention, or a pharmaceutical composition according to the second aspect of the invention.

The term ‘effective amount’ is used herein to describe concentrations or amounts of compositions or pharmaceutical compositions according to the present invention which may be used to produce a favourable change in a disease or condition treated, whether that change is a remission, a favourable physiological result, a reversal or attenuation of a disease state or condition treated, the prevention or the reduction in the likelihood of a condition or disease state occurring, depending upon the disease or condition treated. Where compositions or pharmaceutical compositions of the invention are used in combination, each of the compositions or pharmaceutical compositions may be used in an effective amount, wherein an effective amount may include a synergistic amount.

It will be appreciated by persons skilled in the art that the compositions and pharmaceutical formulations of the present invention have utility in both the medical and veterinary fields. Thus, the methods of the invention may be used in the treatment of both human and non-human animals (such as horses, dogs and cats). Preferably, however, the patient is human.

For veterinary use, a composition of the invention is administered as a suitably acceptable formulation in accordance with normal veterinary practice and the veterinary surgeon will determine the dosing regimen and route of administration which will be most appropriate for a particular animal.

A sixteenth aspect of the invention provides a method for killing microorganisms in vitro comprising contacting the microorganisms with a composition according to the first aspect of the invention or a pharmaceutical composition according to the second aspect of the invention. For example, the composition or pharmaceutical composition may also be used in the form of a sterilising solution or wash to prevent the growth of microorganisms on a surface or substrate, such as in a clinical environment (e.g. surgical theatre) or a domestic environment (e.g. a kitchen work surface, washing clothes such as bed linen).

In one embodiment the antimicrobial compound may be in solution at a concentration of 1 to 100 μg/ml.

In one embodiment the solution further comprises a surface-active agent or surfactant. Suitable surfactants include anionic surfactants (e.g. an aliphatic sulphonate), amphoteric and/or zwitterionic surfactants (e.g. derivatives of aliphatic quaternary ammonium, phosphonium and sulfonium compounds) and nonionic surfactants (e.g. aliphatic alcohols, acids, amides or alkyl phenols with alkylene oxides)

Conveniently, the surface-active agent is present at a concentration of 0.5 to 5 weight percent.

The sterilising solutions are particularly suited for use in hospital environments. For example, the sterilising solutions may be used to sterilise surgical instruments and surgical theatre surfaces, as well as the hands and gloves of theatre personnel. In addition, the sterilising solutions may be used during surgery, for example to sterilise exposed bones. In all cases, the solution is applied to the surface to be sterilised.

The composition or pharmaceutical composition may also be used to disinfect blood and blood products and in the diagnosis of bacterial contamination or infection.

In both in vitro and in vivo uses, the pharmaceutical composition or at least one of the polypeptides is preferably exposed to the target microorganisms (or surface/area to be treated) for at least five minutes. For example, the exposure time may be at least 10 minutes, 20 minutes, 30 minutes, 40 minutes, 50 minutes, 1 hour, 2 hours, 3, hours, 5 hours, 12 hours and 24 hours.

In one embodiment of the composition or pharmaceutical composition for use according to the ninth, tenth or eleventh aspects of the invention, the use according to the twelfth or thirteenth aspects of the invention, or the method according to the fourteenth, fifteenth or sixteenth aspects of the invention, the composition or pharmaceutical composition is coated or impregnated onto, or admixed or otherwise associated with, a medical device, implant, wound care product, or material for use in the same.

The listing or discussion of an apparently prior-published document in this specification should not necessarily be taken as an acknowledgement that the document is part of the state of the art or is common general knowledge.

Preferred, non-limiting examples which embody certain aspects of the invention will now be described, with reference to the following figures:

### DESCRIPTION OF THE FIGURES

FIG. 1: Quantitative evaluation of the in vivo wound healing efficiency of collagen VI-derived peptides in WOUNDCOM in non-infected wounds. In a murine animal model dermal skin, wounds were surgically inflicted. They were subsequently covered with a collagen I carrier impregnated with different collagen VI peptides. Wound healing during a 10 days period was evaluated and compared to natural wound healing, Puracol (carrier without collagen VI peptides) and Puracol Ag (carrier impregnated with silver ions), respectively. Puracol (produced by Medskin) was used at a concentration that has been optimised for clinical performance. The wound reduction rate is considerably accelerated with WOUNDCOM, where the carrier is impregnated with a 1:1 mixture of 3 μM GVR28 and SFV33 (i.e. 1.5 μM of each polypeptide for a total of 3 μM) (FIG. 1A), or a 1:1 mixture of 1.5 μM GVR28 and SFV33 (0.75 μM of each polypeptide for a total of 1.5 μM) (FIG. 1B), respectively.

FIG. 2: Quantitative evaluation of the in vivo wound healing efficiency of collagen VI-derived peptides in WOUNDCOM in infected wounds. In a murine animal model dermal skin wounds were surgically inflicted and infected with 1×10⁶ cfu Pseudomonas aeruginosa. They were subsequently covered with a collagen I carrier impregnated with different collagen VI peptides. Wound healing during a 10 days period was evaluated and compared to natural wound healing, Puracol (carrier without collagen VI peptides) and Puracol Ag (carrier impregnated with silver ions), respectively. The wound reduction rate is considerably accelerated with WOUNDCOM, where the carrier is impregnated with a 1:1 mixture of 3 μM GVR28 and SFV33 (i.e. 1.5 μM of each polypeptide for a total of 3 μM) (FIG. 2A), or a 1:1 mixture of 1.5 μM GVR28 and SFV33 (i.e. 0.75 μM of each polypeptide for a total of 1.5 μM) (FIG. 2B), respectively.

FIG. 3: Analysis of wound exudation as a measure for wound closure. The amount of wound fluid exudation was measured over time in compliance with the scoring system described in httbs://www.woundsinternational.com/resources/details/wuwhs-consensus-document-wound-exudate-effective-assessment-and-management. The wound scoring system is also outlined in the table below.

| Wound | | | |
|---|---|---|---|
| exudate | Extent of | | |
| score | control | Exudate amount | Dressing requirement |
| 1 | Full | None/minimal/ | No absorptive dressings |
| | | | required. If clinically, |
| | | | feasible dressing could |
| | | | stay on for up to a week |
| 2 | Partial | Moderate amount | Dressing changes required |
| | | | every 2-3 days |
| 3 | Uncontrolled | Very exudative | Absorptive dressing changes |
| | | wound | required at least daily |

In summary, wound bed exudation was scored according to the scoring system above, with further divisions, resulting in: 1.1. no exudation, 1.2 minimal exudation, 2.1 moderate exudation, 2.2 moderate to exudative, 3.1 exudative to very exudative, 3.2 very exudative. Wounds were treated with different articles as shown in the Figure. Results are expressed as the mean±SEM of 6 different wounds on 4 animals. Aside from the “Natural wound healing” condition, all other conditions were in infected wounds.

FIG. 4: Analysis of wound bed depth as a measure for wound healing. The depth of the wound bed was measured over time. In each wound the deepest position was measured. Results are expressed as the mean±SEM of 6 different wounds on 4 animals. Aside from the “Natural wound healing” condition, all other conditions were in infected wounds.

FIG. 5: Analysis of wound bed tissue type as a measure for wound healing. The wound bed tissue type was measured over time in compliance with the scoring system described in https://www.coloolastsci/Documents/Wound/WUWHS_POSITION%20DOCUMENT.pdf. In short, tissue types were scored as: 1.1 very sloughy, 1.2 very sloughy to moderately sloughy, 2.1 moderately sloughy to granulating, 2.2 granulating, 3.1 granulating to epithelializing, 3.2 epithelializing. Wounds were treated with different articles as shown in the Figure. Results are expressed as the mean±SEM of 6 different wounds on 4 animals. Aside from the “Natural wound healing” condition, all other conditions were in infected wounds.

FIG. 6: Assessment of bacterial load in the wound. Full thickness wounds were infected with 5×10⁵ cfu Pseudomonas aeruginosa. CFU were quantitatively analysed by viable count assays. Wounds were treated with different articles as shown in the Figure. Notably, in uninfected control wounds (black lines) a secondary infection was observed between days 4 and 7. Results are expressed as the mean±SEM of 6 different wounds on 4 animals.

FIG. 7: Effect on internal collagen fibril structure of WOUNDCOM produced by Manufacturer A. Densely packed collagen fibrils are visible with cross-striation pattern (arrows). WOUNDCOM patches were assessed after 0 months (a), 2 months (b), 6 months (c) and 12 months (d) of shelf life. No structural differences are observed over time.

FIG. 8: Effect of shelf-life on internal collagen fibril structure of WOUNDCOM produced by Manufacturer B. Densely packed collagen fibrils are visible with cross-striation pattern (arrows). WOUNDCOM patches were assessed after 0 months (a) and 2 months (b) of shelf life. No structural differences are observed over time.

FIG. 9: Effect of shelf-life on collagen sponge structure of WOUNDCOM produced by Manufacturer A. WOUNDCOM patches were assessed after 0 months (a), 2 months (b), 6 months (c) and 12 months (d) of shelf life. No structural differences are observed over time.

FIG. 10: Effect of shelf-life on collagen sponge structure of WOUNDCOM produced by Manufacturer B. WOUNDCOM patches were assessed after 0 months (a), 2 months (b) of shelf life. No structural differences are observed over time.

FIG. 11: Effect of shelf-life on collagen VI peptide GVR28 distribution in WOUNDCOM produced by Manufacturer A. WOUNDCOM patches were assessed by gold-labelled antibodies against GVR28 on thin sections. Time points taken were after 0 months (a), 2 months (b), 6 months (c) and 12 months (d) of shelf life. No structural differences were observed over time.

FIG. 12: Effect of shelf-life on collagen VI peptide GVR28 distribution in WOUNDCOM produced by Manufacturer B. WOUNDCOM patches were assessed by gold-labelled antibodies against GVR28 on thin sections. Time points taken were after 0 months (a) and 2 months (b) of shelf life. No structural differences were observed over time.

FIG. 13: Effect of shelf life on collagen VI peptide SFV33 distribution in WOUNDCOM produced by Manufacturer A (a to d) or Manufacturer B (e and f). WOUNDCOM patches were assessed by gold-labelled antibodies against SFV33 on thin sections. For WOUNDCOM produced by Manufacturer A, time points taken were after 0 months (a), 2 months (b), 6 months (c) and 12 months (d) of shelf life. For WOUNDCOM produced by Manufacturer B, time points taken were after 0 months (e) and 2 months (f) of shelf life. No structural differences were observed over time.

FIG. 14: The in vitro effect of different membranes on wound healing in vitro. The effect of three different membranes (WOUNDCOM, ProHeal and Whatman cellulose filter paper) on wound healing was assessed by measuring fibroblast density over a 96 hour period. Cell density (%) was measured at 0 hours, 24 hours, 48 hours and 96 hours. WOUNDCOM (comprising a collagen scaffold and the bioactive collagen VI peptides GVR28 and SFV33) exhibited superior wound healing effects at all time points.

FIG. 15: The in vitro effect of different membranes on bacterial survival. The effect of three different membranes (WOUNDCOM, ProHeal and Whatman cellulose filter paper) on the survival of different bacteria (Staphylococcus aureus or Pseudomonas aeruginosa) was assessed over time. Survival was measured at 0 minutes, 30 minutes, 60 minutes and 120 minutes. WOUNDCOM (comprising a collagen scaffold and the bioactive collagen VI peptides GVR28 and SFV33) exhibited antimicrobial activity at 30, 60 and 120 minutes.

### EXAMPLES

### Example 1—Improved Wound Healing Properties of Combinations of Bioactive Collagen VI Peptides

Introduction

Biomaterials are placed internally to maintain or replace human body functions. They are constructed of various combinations of metal alloys, ceramics, polymers, or biopolymers due to their excellent mechanical properties, corrosion resistance, and biocompatibility. Wound matrices come in a variety of materials including natural polymers and synthetic polymers manufactured into various forms, such as foams, films, hydrocolloids, hydrogels, sponges, membranes, skin substitutes, electro spun micro- and nanofibers. Bioactive wound matrices deliver substances active in wound healing either by delivery of bioactive compounds or by being constructed from materials having endogenous activity. The healing success rate is highly determined by cellular and physiological processes taking place at the host-biomaterial interface during wound healing. Specifically, adverse host response processes often lead to chronic inflammation and encapsulation that preclude the performance of the biomaterial. Hence, it is important to design appropriate wound treatment strategies with the ability to work actively with wound properties such as tissues and cells to enhance healing. Also, patients often suffer from severe infections at the implant site, precluding the normal wound healing process. Usually, harmless bacteria like Staphylococci, Pseudomonas, or Streptococci can infiltrate the damaged tissue in the fresh wound. Here they may develop a high pathogenic potential and establish persistent infections, severely compromising biomaterial function. Therefore, new strategies including bioactive wound healing promoting biomaterial surfaces with antimicrobial effects can be beneficial for the patient. In particular, combinations of different bioactive biomolecules, which allow loading a given biomaterial surface with a spectrum of synergistic effects, are crucial for premium biomaterial function and biocompatibility.

To achieve this goal, the effect of modifying collagen I matrices was investigated (WOUNDCOM Carrier, i.e. Puracol) with a combination of the bioactive peptides GVR28 and SFV33 derived from the collagen VI sequence (WOUNDCOM Effectors). They were compared to WOUNDCOM Carrier alone (Puracol), or WOUNDCOM Carrier modified with GVR28 and SFV33 alone, or with silver ions (Puracol Ag), respectively. The different WOUNDCOM variants were applied in vivo on different skin wounds in a murine model. The combination of GVR28 and SFV33 exhibited an unexpected, particularly high wound healing and inflammation modulatory efficiency, resulting in significantly accelerated wound closure. Together WOUNDCOM carriers and WOUNDCOM effectors are combined to make the WOUNDCOM product.

In summary, these data show that the tested combinations of bioactive collagen VI peptides for WOUNDCOM modifications exhibit particularly high wound healing efficiencies. They may thus promote early and intermediate cellular events on the host-biomaterial interface at a particular high rate as compared to other biomolecules. Thus, combinations of collagen VI derived peptides, bound to a given collagen I wound matrix, may be considered biologically appropriate for premium biocompatibility and tissue integration of the bioactive host-biomaterial interface. In particular, they may protect the wound bed against local infections and the patient against systemic infections after biomaterial insertion/application and during the initial steps of wound closure and wound healing. This treatment strategy will be beneficial for the wound environment, with the potential to promote improved wound repair and reduce abnormal scar formation.

Therefore, in the present study, the use of combinations of native collagen VI-derived biomolecules to promote superior wound healing properties on the surface of different commercially available collagen scaffolds was explored. Here, it is demonstrated for the first time that the use of combinations of native collagen VI-derived biomolecules greatly enhances the wound healing properties of biological collagen scaffolds in vivo. This effect will result in a pronounced potential to provide a versatile, multifunctional, and appropriate extracellular environment, able to actively contrast the onset of infections and inflammation, while promoting tissue regeneration and scar remodeling, and consequently deliver the desired enhancement in biocompatibility.

Materials and Methods

Materials—MDS Collagen and MDS collagen Ag (i.e. Puracol and Puracol Ag, respectively) were obtained from MedSkin Solutions Dr. Suwelack A G (MDS). ε-poly-L-lysine hydrobromide (PLL, 30 000-150 000 g/mol) was purchased from Sigma-Aldrich, St. Louis, USA. Collagen VI and its derived peptides were prepared as described in [29].

Biomaterial surface coating with PLL—Collagen scaffold discs with a diameter and thickness of 5 mm (thick scaffold) and 1-2 mm (thin scaffold), respectively, were punched out from a larger sheet (approximately 10 cm×10 cm). Prior to collagen coating, 150 μl poly-L-lysine hydrobromide solution (0.2 mg/ml) were applied on the collagen scaffold discs by incubation at 60° C. for 2 h. Afterwards the discs were washed twice in distilled water to remove unbound PLL, air-dried and stored at room temperature.

Biomaterial surface coating with bioactive collagen VI molecules—Collagen scaffold discs, after prior treatment with PLL, were put into 24-well cell culture plates (TPP, Trasadingen, Switzerland). They were coated by incubation with 150 μl collagen type VI peptides GVR28, or SFV33, or mixtures of both peptides (concentrations 3 μM or 1.5 μM, respectively) at 4° C. for 2 h, followed by rinsing with distilled water and air-drying.

Animals and husbandry—Balb/c mice (females, 8-10 weeks) were acquired from Janvier Europe. Mice were housed in the animal facility at Medicon Village, Lund, Sweden, and kept at 12 h light/dark cycles in polystyrene cages (type III cages, 10 mice per cage) containing wood shavings and fed standard rodent chow and water ad libitum. Individual mice were identified by ear markings performed at day 1.

Ethical permits—The local animal ethics committee Malmo/Lund, Sweden, approved the experiments under the license 5490-2017.

Biomaterial disk implantation—Mice were anesthetized with Isofluran and shaved on the back using a razor. If necessary, hair removal solution was applied for removal of any remaining hair. The area of implantation was cleaned with iodine or 70% ethanol and administered locally with Marcaine before an incision was made. The coated biomaterial disk was inserted subcutaneously, and the wound was closed with sutures and/or surgical glue.

Termination and tissue collection—The animals were terminated at different time points after implantation, i.e. 1 hour (Tables 2 and 5; non-infected and infected, respectively), 3 days (Tables 3 and 6; non-infected and infected, respectively) and 10 days (Tables 4 and 7; non-infected and infected, respectively). At termination, the animals were perfusion-fixed with ice cold formalin in physiological saline. The implants were collected with the surrounding tissues, transferred to fixative, and stored at 4° C. They were then subjected to standard embedding and immunohistochemistry procedures [38-41].

Results

The combination of the bioactive peptides GVR28 and SFV33 mediates superior wound healing efficiency of collagen I scaffolds in non-infected and infected skin wounds.

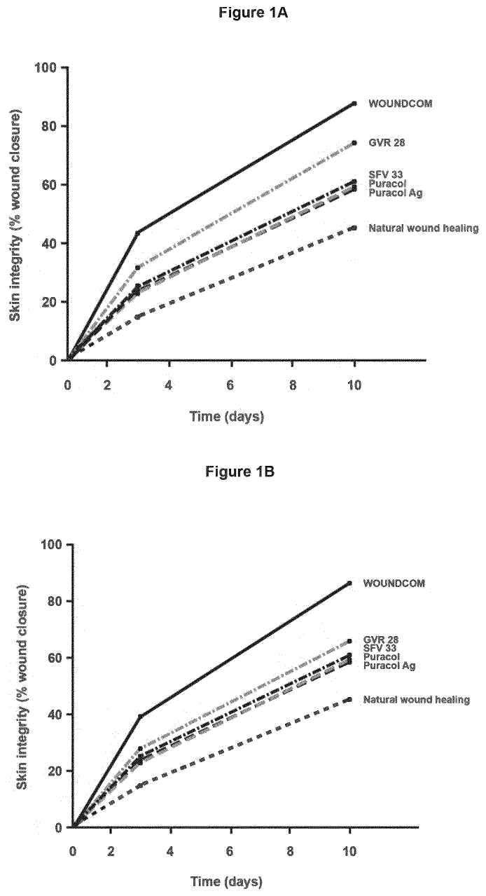

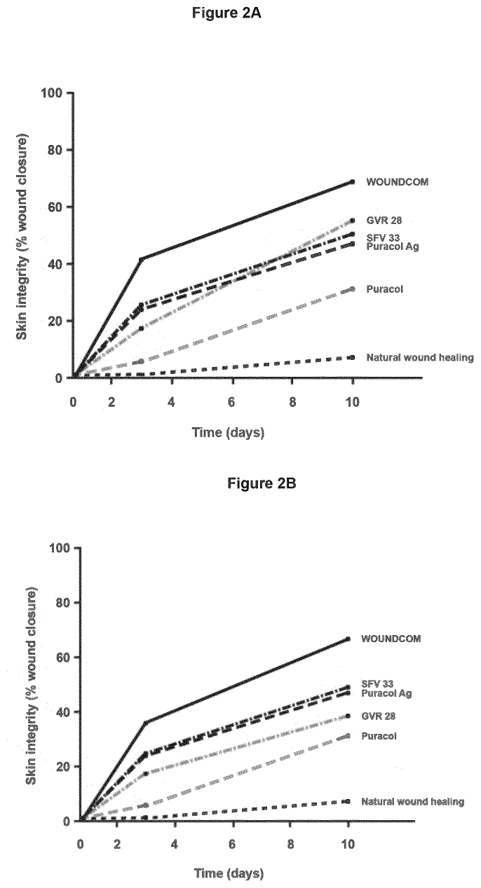

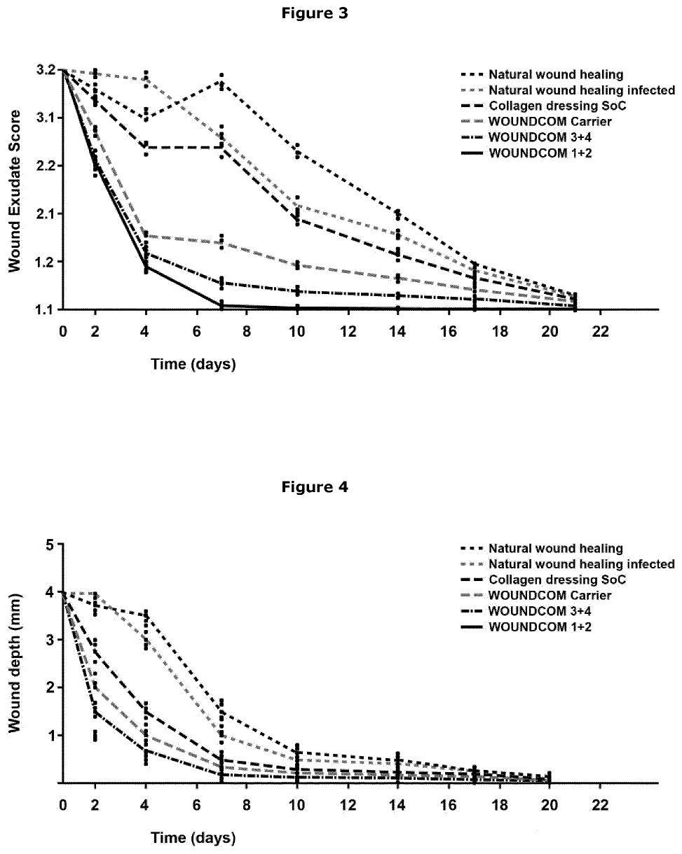

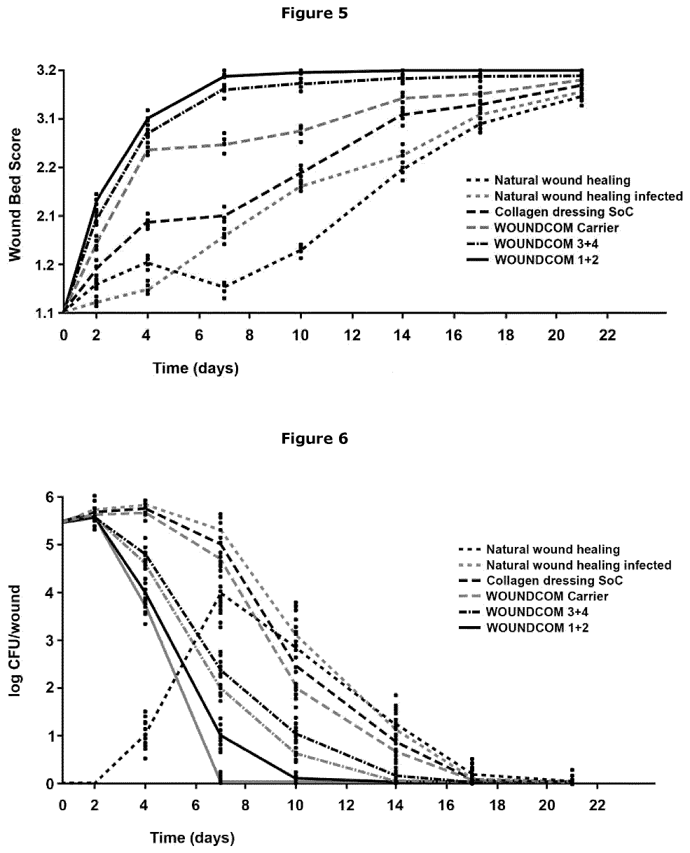

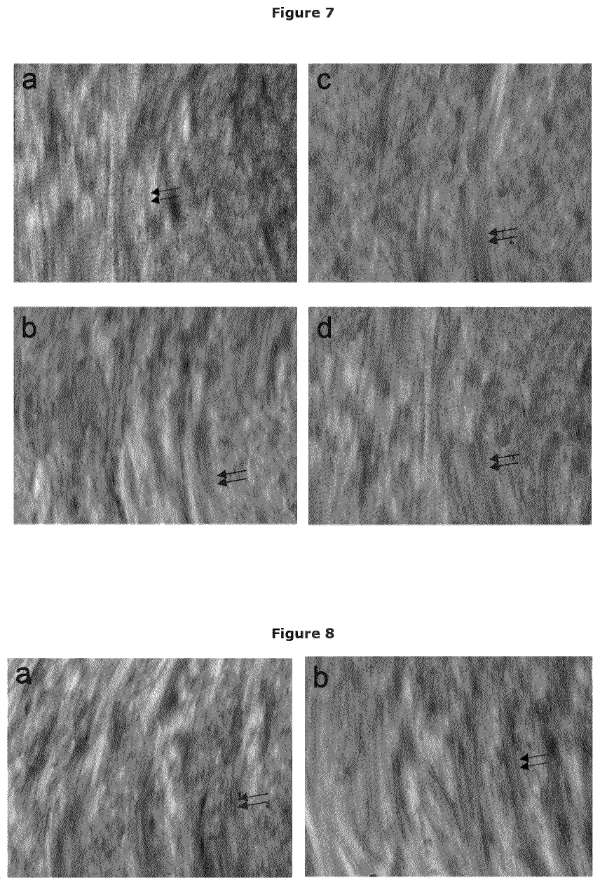

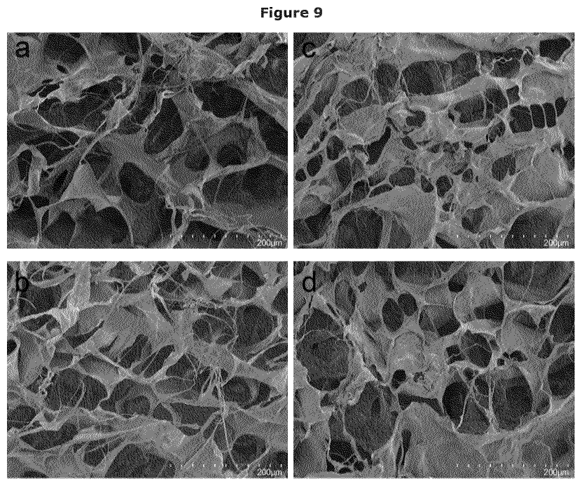

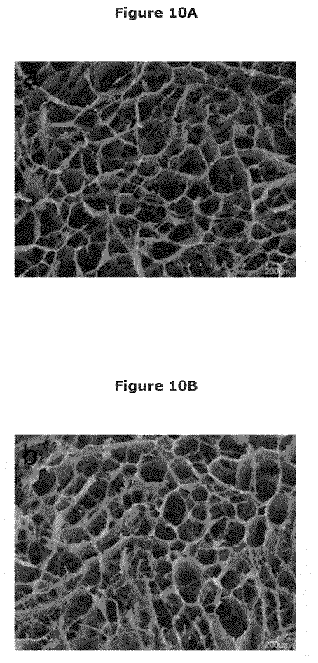

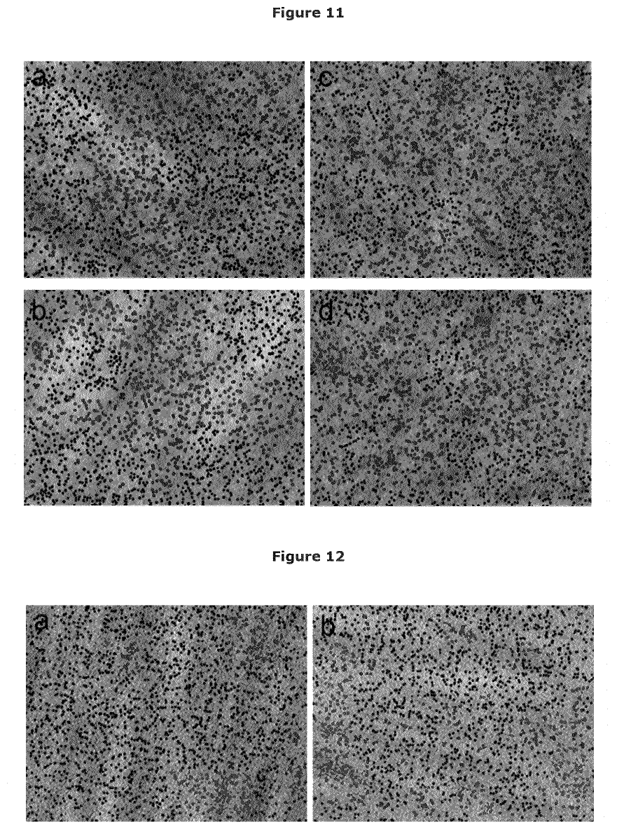

In order to assess possible effects of bioactive collagen VI peptides on wound healing in a murine model, collagen I scaffolds were impregnated with different combinations of the bioactive peptides GVR28 and SFV33. Coating with GVR28 or SFV33 alone, or with silver ions, or collagen scaffolds without prior coating served as controls. The results from histological assessment of murine skin wounds treated with different collagen scaffolds show their different wound healing acceleration properties (FIGS. 1 and 2). Pre-treatment of the collagen I scaffolds with combinations of GVR28 and SFV33 significantly enhanced the wound healing properties in vivo as compared to the controls, both in non-infected wounds and infected wounds (FIGS. 1 and 2, respectively). In addition, the inflammatory responses in the wound bed were modulated in a most beneficial way by collagen I scaffolds with combinations of GVR28 and SFV33 (Tables 2 to 7). Notably, the wound healing efficiency of these collagen I scaffolds was considerably enhanced as compared to the widespread, commercially available gold standard with silver ions.

Taken together, the data presented in FIGS. 1 and 2 and in Tables 2 to 7 show that appropriate combinations of bioactive peptides derived from the collagen VI alpha-3 chain strongly stimulate dermal wound healing in vivo. This superior wound healing effect of collagen I wound matrix, impregnated with GVR28 and SFV33 combinations, was considerably less pronounced for the other tested wound matrices. These effects are expected to result in superior wound healing properties of biomaterials coated with combinations of peptides derived from collagen VI.

Tables 2, 3 and 4: Quantitative assessment of cellular and histological parameters of non-infected wounds treated with WOUNDCOM. In a murine animal model dermal skin wounds were surgically inflicted. They were subsequently covered with a collagen I carrier impregnated with different collagen VI peptides. Wound healing during a 10 days period was evaluated and compared to natural wound healing, Puracol (carrier without collagen VI peptides) and Puracol Ag (carrier impregnated with silver ions), respectively. Table 2: 1 h treatment; table 3: 3 d treatment; table 4: 10 d treatment. The time course of the appearance of different cells in the wound during healing and other wound parameters were quantitatively assessed by histological evaluation. The level of inflammatory response, represented as macrophage, neutrophil and leucocyte counts is significantly reduced in wounds treated with WOUNDCOM, where the carrier is impregnated with a 1:1 mixture of 3 μM GVR28 and SFV33 (i.e. 1.5 μM of each polypeptide for a total of 3 μM). In parallel, the levels of tissue necrosis and fibrin exudation are also significantly reduced in wounds treated with WOUNDCOM, where the carrier is impregnated with a 1:1 mixture of 3 μM GVR28 and SFV33 (i.e. 1.5 μM of each polypeptide for a total of 3 μM).

Tables 5, 6 and 7: Quantitative assessment of cellular and histological parameters of infected wounds treated with WOUNDCOM. In a murine animal model dermal skin wounds were surgically inflicted and infected with 1×10⁶ cfu Pseudomonas aeruginosa. They were subsequently covered with a collagen I carrier impregnated with different collagen VI peptides. Wound healing during a 10 days period was evaluated and compared to natural wound healing, Puracol (carrier without collagen VI peptides) and Puracol Ag (carrier impregnated with silver ions), respectively. Table 5: 1 h treatment; table 6: 3 d treatment; table 7: 10 d treatment. The time course of the appearance of different cells in the wound during healing and other wound parameters were quantitatively assessed by histological evaluation. The level of inflammatory response, represented as macrophage, neutrophil and leucocyte counts is significantly reduced in wounds treated with WOUNDCOM, where the carrier is impregnated with a 1:1 mixture of 3 μM GVR28 and SFV33 (i.e. 1.5 μM of each polypeptide for a total of 3 μM). In parallel, the levels of tissue necrosis and fibrin exudation are also significantly reduced in wounds treated with WOUNDCOM, where the carrier is impregnated with a 1:1 mixture of 3 μM GVR28 and SFV33 (i.e. 1.5 μM of each polypeptide for a total of 3 μM).

TABLE 2
1 hour treatment, non-infected model.

| Treatment 1 h | | | | | | |
|---|---|---|---|---|---|---|
| 3 micromolar | | | | | | |
| Effector | % Effect | | | | | |
| Wound | | | | | Fibrin/scab | Wound |
| dressing | PMN | MΦ | Leucocytes | Necrosis | deposits | closure |
| Control | 3.2 | 0.4 | 5.8 | 0 | 0 | 100 |
| (healthy skin) | | | | | | |
| Control | 36.7 | 8.7 | 62.3 | 0 | 81.3 | 1.2 |
| (wounded skin) | | | | | | |
| Puracol | 25.7 | 6.4 | 45.5 | 0 | 58.2 | 1.2 |
| Puracol Ag | 23.8 | 6.0 | 43.8 | 0 | 57.0 | 1.1 |
| Puracol + GVR28 | 21.9 | 5.4 | 34.3 | 0 | 53.7 | 1.3 |
| Puracol + SFV33 | 21.1 | 5.9 | 38.8 | 0 | 54.7 | 1.4 |
| Puracol + | 19.5 | 4.9 | 33.6 | 0 | 39.6 | 1.2 |
| GVR28 + SFV33 | | | | | | |

TABLE 3
3 day treatment, non-infected model.

| Treatment 3 d | | | | | | |
|---|---|---|---|---|---|---|
| 3 micromolar | | | | | | |
| Effector | % Effect | | | | | |
| Wound | | | | | Fibrin/scab | Wound |
| dressing | PMN | MΦ | Leucocytes | Necrosis | deposits | closure |
| Control | 12.7 | 7.2 | 2.1 | 0 | 0 | 100 |
| (healthy skin) | | | | | | |
| Control | 624.8 | 374.8 | 37.5 | 2 | 55.1 | 13.7 |
| (wounded skin) | | | | | | |
| Puracol | 420.8 | 260.0 | 23.3 | 0.6 | 38.8 | 22.4 |
| Puracol Ag | 429.7 | 271.3 | 25.6 | 0.7 | 38.0 | 22.8 |
| Puracol + GVR28 | 255.6 | 169.0 | 15.1 | 0.3 | 36.4 | 31.9 |
| Puracol + SFV33 | 297.5 | 197.3 | 16.9 | 0.5 | 37.0 | 25.2 |
| Puracol + | 102.3 | 88.7 | 8.8 | 0.1 | 13.1 | 43.1 |
| GVR28 + SFV33 | | | | | | |

TABLE 4
10 day treatment, non-infected model.

| Treatment 10 d | | | | | | |
|---|---|---|---|---|---|---|
| 3 micromolar | | | | | | |
| Effector | % Effect | | | | | |
| Wound | | | | | Fibrin/scab | Wound |
| dressing | PMN | MΦ | Leucocytes | Necrosis | deposits | closure |
| Control | 3.2 | 10.2 | 0.9 | 0 | 0 | 100 |
| (healthy skin) | | | | | | |
| Control | 31.3 | 497.2 | 18.2 | 2 | 27.3 | 44.8 |
| (wounded skin) | | | | | | |
| Puracol | 21.0 | 340.1 | 12.1 | 0.4 | 18.0 | 59.1 |
| Puracol Ag | 20.1 | 283.5 | 12.0 | 0.3 | 18.0 | 57.6 |
| Puracol + GVR28 | 16.8 | 231.1 | 7.2 | 0.3 | 14.1 | 73.9 |
| Puracol + SFV33 | 19.6 | 272.6 | 8.4 | 0.5 | 16.1 | 61.1 |
| Puracol + | 7.8 | 121.0 | 3.1 | 0.1 | 7.6 | 87.7 |
| GVR28 + SFV33 | | | | | | |

TABLE 5
1 hour treatment, infected model.

| Treatment 1 h | | | | | | |
|---|---|---|---|---|---|---|
| 3 micromolar | | | | | | |
| Effector | % Effect | | | | | |
| Wound | | | | | Fibrin/scab | Wound |
| dressing | PMN | MΦ | Leucocytes | Necrosis | deposits | closure |
| Control | 12.4 | 2.9 | 40.1 | 0 | 0 | 100 |
| (healthy skin) | | | | | | |
| Control | 256.9 | 67.7 | 442.9 | 0 | 92.7 | 1.2 |
| (wounded skin) | | | | | | |
| Puracol | 185.0 | 45.9 | 388.5 | 0 | 72.3 | 1.1 |
| Puracol Ag | 179.9 | 41.6 | 338.9 | 0 | 61.2 | 1.2 |
| Puracol + GVR28 | 149.1 | 36.4 | 260.3 | 0 | 56.0 | 1.3 |
| Puracol + SFV33 | 131.7 | 31.1 | 246.8 | 0 | 55.1 | 1.2 |
| Puracol + | 127.5 | 24.9 | 175.2 | 0 | 40.4 | 1.2 |
| GVR28 + SFV33 | | | | | | |

TABLE 6
3 day treatment, infected model.

| Treatment 3 d | | | | | | |
|---|---|---|---|---|---|---|
| 3 micromolar | | | | | | |
| Effector | % Effect | | | | | |
| Wound | | | | | Fibrin/scab | Wound |
| dressing | PMN | MΦ | Leucocytes | Necrosis | deposits | closure |
| Control | 73.9 | 49.2 | 65.8 | 0 | 0 | 100 |
| (healthy skin) | | | | | | |
| Control | 6810.3 | 4085.2 | 408.7 | 38.9 | 68.1 | 1.6 |
| (wounded skin) | | | | | | |
| Puracol | 4544.7 | 2834.9 | 253.9 | 21.6 | 43.6 | 5.1 |
| Puracol Ag | 4183.3 | 2739.3 | 239.5 | 20.5 | 46.0 | 23.8 |
| Puracol + GVR28 | 2786.0 | 1742.7 | 161.9 | 10.2 | 41.4 | 17.9 |
| Puracol + SFV33 | 2242.7 | 1664.1 | 152.1 | 10.8 | 40.7 | 26.2 |
| Puracol + | 1115.3 | 976.8 | 98.9 | 3.1 | 25.0 | 42.1 |
| GVR28 + SFV33 | | | | | | |

TABLE 7
10 day treatment, infected model.

| Treatment 10 d | | | | | | |
|---|---|---|---|---|---|---|
| 3 micromolar | | | | | | |
| Effector | % Effect | | | | | |
| Wound | | | | | Fibrin/scab | Wound |
| dressing | PMN | MΦ | Leucocytes | Necrosis | deposits | closure |
| Control | 23.7 | 71.2 | 9.7 | 0 | 0 | 100 |
| (healthy skin) | | | | | | |
| Control | 544.3 | 5469.2 | 18.2 | 19.2 | 47.4 | 7.1 |
| (wounded skin) | | | | | | |
| Puracol | 431.4 | 3842.0 | 12.1 | 11.4 | 35.8 | 31.4 |
| Puracol Ag | 291.2 | 2629.5 | 12.0 | 10.2 | 30.2 | 46.9 |
| Puracol + GVR28 | 215.6 | 2998.4 | 7.2 | 5.3 | 27.1 | 55.9 |
| Puracol + SFV33 | 185.9 | 2542.3 | 8.4 | 6.4 | 24.3 | 51.3 |
| Puracol + | 79.2 | 1331.6 | 3.1 | 0.7 | 13.4 | 69.3 |
| GVR28 + SFV33 | | | | | | |

### Example 2—Improved Wound Healing Properties of Combinations of Bioactive Collagen VI Peptides in an In Vivo Dig Study

Following the positive findings of the bioactive collagen VI peptides on wound healing in a murine model, the bioactive collagen VI peptides were assessed on wound healing in a porcine model, which more closely correlates with wound healing in human.

The assessments were conducted using the following WOUNDCOM compositions:
WOUNDCOM 1: 3 uM of Effector (1.5 uM GVR28+1.5 uM SFV33), no PLL
WOUNDCOM 2: 30 uM of Effector (15 uM GVR28+15 uM SFV33), no PLL
WOUNDCOM 3: 3 uM of Effector (1.5 uM GVR28+1.5 uM SFV33), containing PLL
WOUNDCOM 4: 30 uM of Effector (15 uM GVR28+15 uM SFV33), containing PLL

Wound Closure Measured by Assessment of Wound Exudation

Wound exudate (also referred to as wound fluid or wound drainage) refers to material composed of serum, fibrin and white blood cells that escape into a superficial lesion or area of inflammation (as defined in Merriam-Webster Dictionary, 2018), and plays an important role in wound healing, but can delay healing when in the wrong amount, place or composition. Wound exudate supports the healing process by providing a moist wound environment, enabling immune mediators and growth factors to diffuse across the wound bed, acting as a medium for migrations of tissue-repairing cells, supplying essential nutrients for cell metabolism, and promoting autolysis.

The scoring system for assessment of wound exudation is described in World Union of Wound Healing Societies (WUWHS) Consensus Document, Wound exudate: effective assessment and management, Wounds International, 2019.

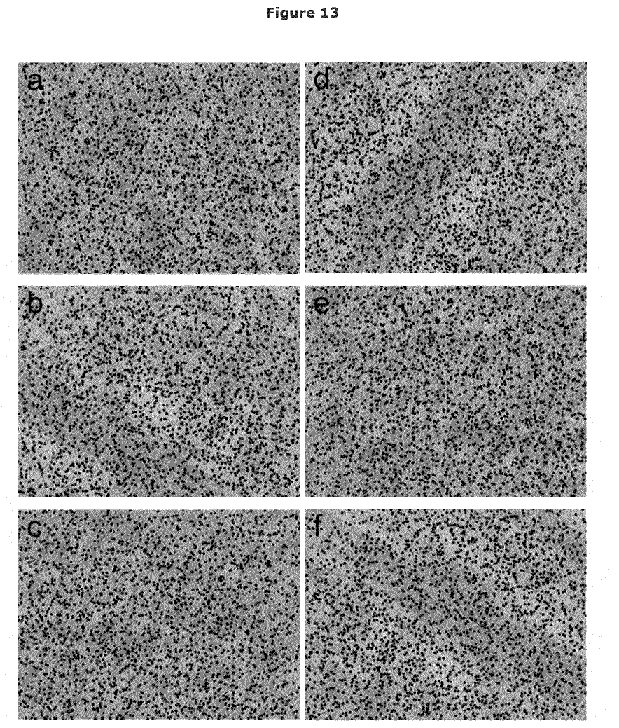

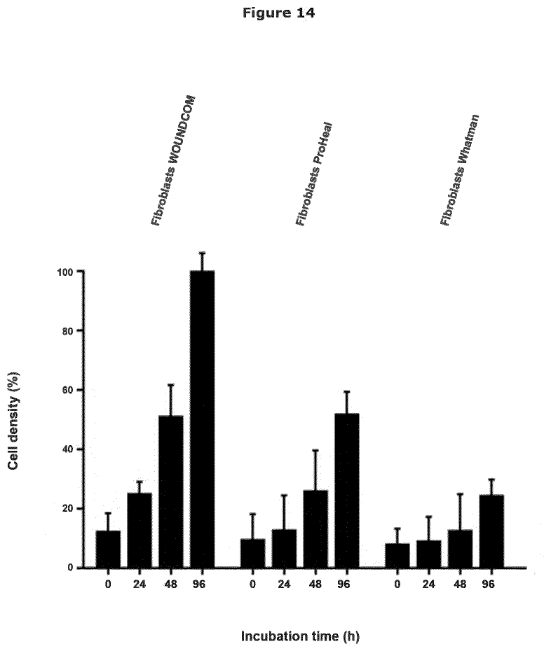

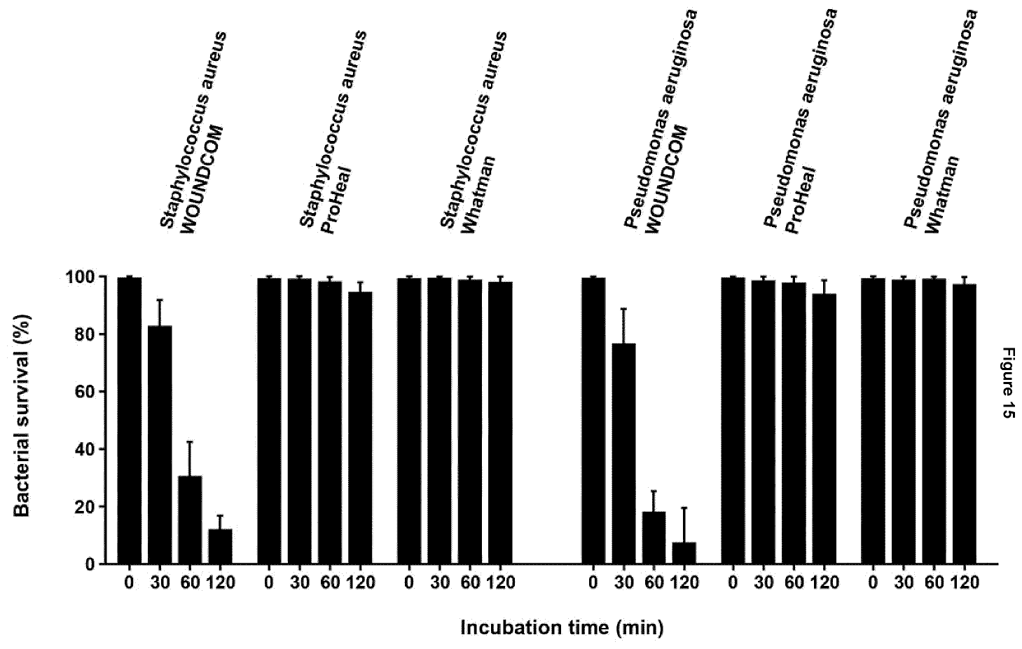

Accordingly, wound healing can be assessed with respect to wound exudate using criteria established in a consensus document by the World Union of Wound Healing Societies, as demonstrated in FIG. 3.

The wound scoring system is also outlined in the table below.

| Wound | | | |
|---|---|---|---|
| exudate | Extent of | | |
| score | control | Exudate amount | Dressing requirement |
| 1 | Full | None/minimal/ | No absorptive dressings |
| | | | required. If clinically, |
| | | | feasible dressing could |
| | | | stay on for up to a week |
| 2 | Partial | Moderate amount | Dressing changes required |
| | | | every 2-3 days |
| 3 | Uncontrolled | Very exudative | Absorptive dressing changes |
| | | wound | required at least daily |

FIG. 3 demonstrates that WOUNDCOM (which is the collagen I wound matrix, impregnated with GVR28 and SFV33 combinations, as described in Example 1) without polylysine (WOUNDCOM 1+2) has an improved refractory period for wound exudate, reducing the score to healthier levels at a faster rate and to a more improved rate compared with all other conditions tested. WOUNDCOM with polylysine (WOUNDCOM 3+4) was also vastly improved, although not quite to the same level as WOUNDCOM 1+2.

Wound Closure Measured by Assessment of Wound Depth

Another assessment of wound closure includes a measurement of wound depth. A wound is inflicted on the pig and its depth is then measured (e.g. in millimetres). Further measurements of the depth are taken at regular time intervals to assess how quickly a wound heals, wherein a reduction in depth correlates with improved healing.

FIG. 4 demonstrates that the WOUNDCOM 1+2 and 3+4 provide the fastest reduction in wound depth (i.e. the fastest wound healing), with complete wound healing being reached by 7 days for WOUNDCOM 1+2. The experiment was terminated at 21 days, by which time natural wound healing, infected wound healing and collagen dressing SoC had still not reached the same level of wound healing, as assessed by wound depth, as compared with WOUNDCOM 1+2 and 3+4.

Wound Closure Measured by Assessment of Wound Bed Tissue Type

A further assessment of wound closure includes a measurement of the wound bed tissue type, in particular the Triangle of Wound Assessment approach, which was developed to improve on the many wound assessment tools previously available. Following a study of 14 wound assessment tools, it was found that while each provided a framework to record certain parameters of wound status, none met all of the criteria for optimal wound assessment and many failed to guide practice in terms of setting goals for healing, planning care and determining critical interventions (Anderson K, Hamm R L. Factors that impair wound healing. 3 Am Coll Clin Wound Specialists 2012; 4(4): 84-91. 11). The Triangle approach is an improved assessment compared with such previous wound assessment criteria.

The scoring system for assessment of wound bed tissue type is described in World Union of Wound Healing Societies (WUWHS), Florence Congress, Advances in wound care: the Triangle of Wound Assessment, Wounds International, 2016 and is outlined in the table above.

FIG. 5 demonstrates once again that WOUNDCOM 1+2 and 3+4 outperform all other conditions for improving the wound bed score more rapidly, and reaching a higher score overall by the end of the experiment.

Assessment of Bacterial Load in the Wound Bed

The antimicrobial properties were also tested for the various WOUNDCOM conditions by infecting full thickness wounds with Pseudomonas aeruginosa, a gram-negative aerobic rod bacterium that is associated with opportunistic infections in the clinic. Antimicrobial activity is demonstrated by a reduction in the number of bacteria, as measured by log colony forming units per wound (log CFU/wound).

FIG. 6 demonstrates that all variations of WOUNDCOM (1, 2, 3 and 4) exhibit improved antimicrobial activity compared with the control conditions, with WOUNDCOM 1 performing best and WOUNDCOM 2, 3 and 4 also exhibiting high antimicrobial properties.

Conclusion

To summarise, it is clear from the data of FIGS. 3-6 that WOUNDCOM provides a safe and effective means to treat infected wounds and WOUNDCOM promotes rapid wound closure and wound healing as compared to other collagen membranes without Effector molecules.

WOUNDCOM 1+2 (without PLL) accelerate wound size reduction more efficiently than WOUNDCOM 3+4 (containing PLL), and no difference in wound size reduction was observed between 3 μM Effector content (WOUNDCOM 1 and 3) and 30 μM Effector content (WOUNDCOM 2 and 4). WOUNDCOM 1 exhibited the highest rate in bacterial load reduction in the wound bed.

Taken together, the data presented in FIGS. 3-6 show that appropriate combinations of bioactive peptides derived from the collagen VI alpha-3 chain strongly stimulate dermal wound healing in vivo in a porcine model, which closely resembles human wound healing. As with the murine studies, the superior wound healing effect of collagen I wound matrix, impregnated with GVR28 and SFV33 combinations (WOUNDCOM), was considerably less pronounced for the other tested wound matrices. These effects are expected to result in superior wound healing properties of biomaterials coated with combinations of peptides derived from collagen VI. This coating strategy will be beneficial for the wound environment, with the potential to promote improved wound repair and reduce abnormal scar formation.

### Example 3—Stability of Collauen VI Peptides (Stand-Alone)

The stability of the collagen VI peptides GVR28 and SFV33 was assessed. By stability we include the purity of the peptides and/or the long-term stability of the peptides at a particular temperature, particularly at −20° C. or 25° C.

i. Initial Stability Tests

Collagen VI-derived peptides GVR28 and SFV33 were synthesized under non-cGMP by a peptide synthesis provider.

The Collagen VI-derived peptides GVR28 and SFV33 were stored for 3 years at either −20° C., or at 25° C. at bench top conditions. The peptides were tested at regular intervals for their wound healing (via in vitro scratch test) and antimicrobial properties (via viable count assays).

Results

The in vitro scratch test has indicated that there are no cytotoxic effects of the peptides on HaCaT cells, and that the wound healing activity of the peptides did not decline during the test period. The viable count assays showed a continuous broad spectrum antimicrobial activity of the peptides, which did not decline throughout the test period.

No significant changes in these parameters were observed during the time period of 3 years at either storage temperature.

ii. Further Stability and Purity Tests

Both GVR28 and SFV33 were synthetized under non-cGMP conditions. The peptides were subject to investigation to evaluate and validate test and analysis methods to assess their purity (chromatography, LC-MS) and assess impurities/related substances generated during peptide synthesis. This assessment is used in material characterization as part of risk management. As background, during synthesis of the peptides GVR28 and SFV33, small amounts of impurities, also mentioned as related substances, are generated as normal part of the process. Related substances are e.g., truncated peptides or peptides including modified amino acids. This fraction is 35 minimized as far as possible. According to the material specification, up to 5% impurities are allowed. During process development, related substances were analysed, and upper limits were defined for all substances at a concentration of ≥0.50%. All impurities ≥0.10% were reported in the Certificate of Analysis.

During the analysis the collagen VI-derived peptides GVR28 and SFV33 were analysed by quantitative chromatography, and impurities were characterized by their relative retention times (RTT), peak areas and calculated molecular weights. The impurities/related substances with suggested molecular structures, based on their calculated molecular weights, were assessed, as well as assessment of potential biocompatibility/toxicity issues.

Both GVR28 and SFV33 peptide preparations are highly pure, with purities of 96% in the Tech batch.

Each peptide preparation contains several impurities/related substances, which could be divided into two groups:

(1) Truncated Peptides with Missing Amino Acids

The truncated peptides are generally considered to be inactive. This is due to the fact that the activity of host defence peptides is highly dependent on their secondary structure, including the correct arrangement of charged and hydrophobic amino acids along the peptide strand [50, 51]. Interestingly, it has also been described that AMPs with increasing length are more destructive to membranes (cytotoxic), which renders truncated peptides even less cytotoxic than the original full-length peptides ([50]).

(2) Peptides with Amino Acid Modifications

All peptide modifications are classified as non-toxic and, thus, do not present any harms to the final product. These findings are based on database searches (ECHA, PubChem). There is, however, one exception, a toxic hypothetical peptide modification, 1,1,3,3-tetramethyl guanidine. Importantly, this substance would be an integral part of the polypeptide chain as an amino acid modification and, thus, not be present in the final product in the free chemical form. Furthermore, it would be present at a very low concentration, far below toxic concentration levels and, hence, not represent any harm to the final product.

Hence, the peptides and impurities checked did not indicate any toxic or biocompatibility issues.

ii. Further Stability Tests

Both GVR28 and SFV33 were synthesized by AmbioPharm Inc. under cGMP conditions. The individual peptides GVR28 and SFV33 (i.e. not in the WOUNDCOM format) are the subject to ongoing stability testing at both −20° C. and +25° C. These will be conducted for up to 24 months.

The stability studies are performed in accordance with AmbioPharm's internal Standard Operating Procedure (SOP); Slab 11.12-005 “Stability Study Management Procedure”, and individual stability protocols; APi3091 (FP.231-3)-001 for GVR28, and APi3092(FP.232-3)-001 for SFV33.

The SOP references the following applicable documents for the planning and conduct of stability studies for APIs:
Guidance for industry Q1A (R2) Stability Testing of New Drug Substances and Products
Guidance for Industry Q1E Evaluation of Stability Data
ICH Q7—Good Manufacturing Practice Guidance for Active Pharmaceutical Ingredients, Subpart 11.5 “Stability monitoring of APIs”
Eudralex Volume 4, Part II: Section 11.5 “Stability Monitoring of APIs”
Chinese Pharmacopoeia (2020 Edition) Volume IV, 9001-Stability testing of active pharmaceutical ingredients and finished pharmaceutical products

Results

Below are experimental results from stability testing at day 0 (0 D) and three months. Tables 8 and 9 show the stability results for storage of GVR28 at −20° C. and 25° C. respectively. Tables 10 and 11 show the stability results for storage of SFV33 at −20° C. and 25° C. respectively. 25° C. was selected to represent accelerated conditions.

This research is believed to provide useful information and indications about long-term peptide stability and potential hazards.

TABLE 8
Stability results for GVR28 (SEQ ID NO: 1) at −20° C., 0 and 3 months
−20 ± 5° C.

| Test | Protocol | | 0 D | | | 3 months | | |
|---|---|---|---|---|---|---|---|---|
| Appearance | White to off-white powder | | White powder | | | White powder | | |
| Identity | Retention Time Conforms | | Retention Time Conforms | | | Retention Time Conforms | | |
| (RP-HPLC) | with Reference Standard | | with Reference Standard | | | with Reference Standard | | |
| Purity | ≥95% | | 98.10% | | | 98.10% | | |
| (RP-HPLC) | | | | | | | | |
| Related | Specified | RRT = 0.82 ≤ 1.50% | RRT = 0.80* | RRT = 0.96* | RRT = 1.04 | RRT = 0.83* | RRT = 0.97 | RRT = 1.04 |
| Impurities | Impurity % | RRT = 0.97 ≤ 0.50% | RRT = 0.81* | 0.38% | 0.56% | 0.04% | 0.38% | 0.54% |
| (by RRT) | | RRT = 1.04 ≤ 0.70% | Total | | | | | |
| | | RRT = 1.05 ≤ 0.50% | 0.07% | | | | | |
| | | RRT = 1.12 ≤ 1.20% | RRT = 1.05 | RRT = 1.13* | N/A | RRT = 1.05 | RRT = 1.12 | N/A |
| | | | ND | 0.22% | | ND | 0.20% | |
| | Unspecified | ≤0.50% | conforms | | | conforms | | |
| | Impurity % | Report ≥0.10% | RRT = 0.94 | RRT = 1.09 | N/A | RRT = 0.95 | RRT = 1.08 | N/A |
| | | | 0.15% | 0.32% | | 0.17% | 0.31% | |

| | | | |
|---|---|---|---|
| Water | ≤15% | 4.2% | 1.8% |
| Content (KF) | | | |

*RRTs are approximate
Product code: FP.231-1

TABLE 9
Stability results for GVR28 (SEQ ID NO: 1) at +25° C., 0, 1 and 3 months
25 ± 2° C./60% RH ± 5% Stability

| Test | Protocol | | 0 D | | | 1 month | | | 3 months | | |
|---|---|---|---|---|---|---|---|---|---|---|---|
| Appearance | White to off-white | | White powder | | | White powder | | | White powder | | |
| | powder | | | | | | | | | | |
| Identity | Retention Time Conforms | | Retention Time Conforms | | | Retention Time Conforms | | | Retention Time Conforms | | |
| (RP-HPLC) | with Reference Standard | | with Reference Standard | | | with Reference Standard | | | with Reference Standard | | |
| Purity | ≥95% | | 98.1% | | | 97.7% | | | 97.3% | | |
| (RP-HPLC) | | | | | | | | | | | |
| Related | Specified | RRT = 0.82 | RRT = | RRT = | RRT = | RRT = | RRT = | RRT = | RRT = | RRT = | RRT = |
| Impurities | Impurity % | ≤1.50% | 0.80* | 0.96* | 1.04 | 0.82* | 0.97 | 1.04 | 0.83* | 0.97 | 1.04 |
| (by RRT) | | RRT = 0.97 | RRT = | 0.38% | 0.56% | 0.07% | 0.40% | 0.57% | 0.12% | 0.41% | 0.56% |
| | | ≤0.50% | 0.81* | | | | | | | | |
| | | RRT = 1.04 | Total | | | | | | | | |
| | | ≤0.70% | 0.07% | | | | | | | | |
| | | RRT = 1.05 | RRT = | RRT = | N/A | RRT = | RRT = | N/A | RRT = | RRT = | N/A |
| | | ≤0.50% | 1.05 | 1.13* | | 1.05 | 1.12 | | 1.05 | 1.12 | |
| | | RRT = 1.12 | ND | 0.22% | | ND | 0.49% | | ND | 0.77% | |
| | | ≤1.20% | | | | | | | | | |
| | Unspecified | ≤0.50% | Conforms | | | Conforms | | | conforms | | |
| | Impurity % | Report | RRT = | RRT = | N/A | RRT = | RRT = | N/A | RRT = | RRT = | N/A |
| | | ≥0.10% | 0.94 | 1.09 | | 0.95 | 1.08 | | 0.95 | 1.08 | |
| | | | 0.15% | 0.32% | | 0.16% | 0.32% | | 0.17% | 0.32% | |

| | | | | |
|---|---|---|---|---|
| Water | ≤15% | 4.2% | 3.9% | 3.9% |
| Content (KF) | | | | |

*RRTs are approximate
Product code FP.231-3

TABLE 10
Stability results for SFV33 (SEQ ID NO: 5) at −20° C., 0 and 3 months
−20 ± 5° C.

| Test | Protocol (rev. 001) | | 0 D | | | 1 month | | |
|---|---|---|---|---|---|---|---|---|
| Appearance | White to off-white powder | | White powder | | | White powder | | |
| Identity | Retention Time Conforms | | Retention Time Conforms | | | Retention Time Conforms | | |
| (RP-HPLC) | with Reference Standard | | with Reference Standard | | | with Reference Standard | | |
| Purity | ≥95% | | 98.1% | | | 98.3% | | |
| (RP-HPLC) | | | | | | | | |
| Related | Specified | RRT = 0.90 ≤ 1.65% | RRT = 0.90 | RRT = 0.95 | RRT = 0.97 | RRT = 0.90 | RRT = 0.95 | RRT = 0.98 |
| Impurities | Impurity % | RRT = 0.95 ≤ 0.55% | 0.26% | 0.14% | 0.47% | 0.23% | 0.10% | 0.44% |
| (by RRT) | | RRT = 0.98 ≤ 2.30% | | | | | | |

| No other individual | conforms | | | conforms | | |
|---|---|---|---|---|---|---|
| impurity ≥0.50% | | | | | | |
| Report all ≥0.10% | RRT = 0.65 | RRT = 1.02 | N/A | RRT = 1.03 | N/A | N/A |
| | 0.17% | 0.23% | | 0.24% | | |

| | | | |
|---|---|---|---|
| Water | ≤15% | 4.7% | 4.9% |
| Content (KF) | | | |

Product code: FP.232-3

TABLE 11
Stability results for SFV33 (SEQ ID NO: 5) at +25°° C., 0, 1 and 3 months
25 ± 2° C./60% RH ± 5% Stability

| Test | Protocol (rev. 001) | | 0 D | | | 1 month | | | 3 months | | |
|---|---|---|---|---|---|---|---|---|---|---|---|
| Appearance | White to off-white powder | | White powder | | | White powder | | | White powder | | |
| Identity | Retention Time Conforms | | Retention Time Conforms | | | Retention Time Conforms | | | Retention Time Conforms | | |
| (RP-HPLC) | with Reference Standard | | with Reference Standard | | | with Reference Standard | | | with Reference Standard | | |
| Purity | ≥95% | | 98.1% | | | 97.8% | | | 96.9% | | |
| (RP-HPLC) | | | | | | | | | | | |
| Related | Specified | RRT = 0.90 ≤ | RRT = | RRT = | RRT = | RRT = | RRT = | RRT = | RRT = | RRT = | RRT = |
| Impurities | Impurity % | 1.65% | 0.90 | 0.95 | 0.97 | 0.90 | 0.95 | 0.97 | 0.90 | 0.95 | 0.97 |
| (by RRT) | | RRT = 0.95 ≤ | 0.26% | 0.14% | 0.47% | 0.24% | 0.13% | 0.47% | 0.20% | 0.09% | 0.42% |
| | | 0.55% | | | | | | | | | |
| | | RRT = 0.98 ≤ | | | | | | | | | |
| | | 2.30% | | | | | | | | | |

| No other individual | conforms | | | conforms | | | Not conforms * | | |
|---|---|---|---|---|---|---|---|---|---|
| impurity ≥0.50% | | | | | | | RRT = 1.05 0.56% | | |
| | | | | | | | RRT = 1.09 0.53% | | |
| Report all ≥0.10% | RRT = | RRT = | N/A | RRT = | RRT = | RRT = | RRT = | RRT = | N/A |
| | 0.65 | 1.02 | | 0.66 | 0.85 | 1.02 | 0.62 | 0.84 | |
| | 0.17% | 0.23% | | 0.13% | 0.11% | 0.31% | 0.15% | 0.28% | |
| | N/A | N/A | N/A | RRT = | RRT = | N/A | RRT = | RRT = | N/A |
| | | | | 1.04 | 1.08 | | 1.05* | 1.09* | |
| | | | | 0.17% | 0.24% | | 0.56% | 0.53% | |

| | | | | |
|---|---|---|---|---|
| Water | ≤15% | 4.7% | 6.8% | 8.4% |
| Content (KF) | | | | |

Product code: FP.232-3

No degradation was observed between Day 0 and the 3 month point for GVR28 or SFV33 stored at −20° C. A slight decrease in purity was observed for GVR28 and SFV33 stored at 25° C. during the initial 3 months. However, at 3 months the purity level was within the acceptance criteria.

For SFV33 stored at 25° C., some new impurities were detected at 3 months, but this is to be expected when purity decreases. Water content increased over the 3 months, but again was still within the acceptance criteria.

The SFV33-impurities seen at room temperature after 3 months will be evaluated but do not raise any concern, and are anticipated to be harmless truncated and inactive sequences.

The ongoing long-term peptide stability tests show no significant change of the stability of the collagen VI peptides (GVR28 and SFV33) in regard to appearance, identity, purity/related impurities, and water content. The stability studies indicate that the collagen VI peptides will be stable in their freeze-dried state for durations that are commercially and clinically relevant.

### Example 4—Stability of Assembled WOUNDCOM Comprising Collagen VI Peptides

WOUNDCOM was produced by two independent companies and different important product stability parameters were evaluated by electron microscopy, including:
(i) the long-term collagen I fibril and collagen I sponge stability and integrity (in particular, gross appearance and collagen sponge pore sizes); and
(ii) the long-term peptide stability and concentration in the WOUNDCOM matrix via quantitative electron microscopy with gold labelled polyclonal antibodies.

WOUNDCOM Manufacturing Process

In order for stability to be assessed, WOUNDCOM units were manufactured as follows.

Bovine Collagen I

Bovine collagen I was harvested by mechanical means. The dermis was separated from underlying tissues (fat, muscles, bone) and from the epidermis (split skin production) with knives. Tendons were separated mechanically from muscle and bone.

To reduce the risk of TSE/BSE, as well as transfer of other microorganisms that may reside in the raw material, the procedures stipulated in the ISO 22442-series of standards were adhered to in order to ensure proper sourcing, traceability, handling, and control. For example, the herds were BSE free, under constant control, and the tissue harvesting method carefully avoided contact with the body parts that could potentially be TSE-contaminated such as central nervous system tissues.

The processing to a collagen suspension is tough to survive for microorganisms and the resulting collagen suspension had a sufficiently reduced load of microorganisms by default. A viral inactivation study in accordance with ISO 22442-3 validated that the processing steps reduced the viral load sufficiently. Also, the processed collagen was devoid of cells, cell debris, lipids, RNA and DNA.

The workflow ensured a stable and reproducible production of bovine collagen I Human collagen VI peptides GVR28 and SFV33 that were exact replicas of sequences of the alpha 3-chain of human collagen VI. The peptides were present in the product at a concentration of around 3 μM each. This peptide amount has been shown to provide the desired effect. The peptides were synthetically manufactured by standard chemical peptide synthesis. The peptide content of the final freeze-dried material is ≥70%. The remaining content is water and acetate. Of the peptide content, the purity of GVR28 or SFV33, respectively, was in the range of >95%. Any single impurity that exceeded 0.50% was characterised by default by the manufacturer. For GVR28 and SFV33 the major impurities have been analysed and evaluated. They consist of truncated or modified peptides, with impaired or no intact function.

This workflow ensures a stable and reproducible production of the synthetic, bioactive human collagen VI peptides.

WOUNDCOM was manufactured as follows:
1. The collagen VI peptides were chemically synthesized and then freeze dried and packed in glass vials and kept in freezer.
2. The bovine tissues were processed until pure, native collagen I suspension was derived.
3. The peptides were mixed into the collagen slurry mechanically.
4. The resulting suspension was cast into a container, frozen and then lyophilized (freeze-dried).
5. Dehydrothermal Crosslinking (DHT) took place in connection with lyophilization (no chemical crosslinking)
6. Cutting/slicing and packaging of sheets into sterile bags
7. Sterilisation

The final manufactured product comprises of a carrier (Collagen I) and Effector molecules (e.g. GRV28 and SFV33)—which together form the WOUNDCOM product.

WOUNDCOM Stability Experiments

WOUNDCOM prototypes were produced by Manufacturer A and Manufacturer B. They were stored at room temperature and under dry conditions. Samples were examined by electron microscopy at delivery, and then at different time points to assess structural stability aspects of the products.

For this purpose, parameters like collagen fibril structure, nativity, and architecture in different WOUNDCOM specimens were investigated by assessing samples by scanning and transmission electron microscopy. 5 different WOUNDCOM patches were divided into 5 different areas. From each area, 5 different samples were punched out (2 mm diameter), in total 125 samples. The samples were subjected to established routine electron microscopy sample preparation.

Thus, the production stability study was designed to apply established routine methodology of scanning electron microscopy and transmission electron microscopy on WOUNDCOM. This technique proved as a valuable tool to directly assess structural details of the WOUNDCOM scaffold. Thus, the density of native collagen fibres/μm2 was evaluated quantitatively in WOUNDCOM articles from Manufacturer A and Manufacturer B as a measure for material quality directly after manufacturing and during shelf-life ageing (FIGS. 7-10).

In another approach, the production stability study was designed to apply established routine methodology of quantitative immune electron microscopy on WOUNDCOM, which allowed for the direct assessment of local GVR28 and SFV33 concentration and distribution within the WOUNDCOM scaffold. The GVR28 and SFV33 concentration variations throughout different WOUNDCOM scaffolds were evaluated after manufacturing and during shelf-life ageing (FIGS. 11-13). The results serve as an indication of the quality of the prototypes studied, and hence can serve as feedback regarding the manufacturing process parameters. The numbers of immunogold particles (black dots)/μm² directly translate to concentrations of GVR28 and SFV33 peptides.

The results serve as an indication of the quality of the prototypes studied, and hence can serve as feedback regarding the manufacturing process parameters. The studies were executed in compliance with standard procedures and published study protocols.

Results

The effect on internal collagen fibril structure of WOUNDCOM produced by two different manufacturers, Manufacturer A (FIG. 7) and Manufacturer B (FIG. 8), was assessed over time using transmission electron microscopy. All examined WOUNDCOM specimens exhibit a homogenous ultrastructure with a collagen fibril density of about 10-12 collagen fibrils/pmt. The collagen fibrils appear native with a cross-striation periodicity of 63-67 nm. All WOUNDCOM samples exhibit a homogenous ultrastructure with a high density of native collagen I fibrils. The ultrastructural properties of the collagen I templates were independently of the manufacturer and did not change during the tested shelf life.

The effect on collagen sponge structure of WOUNDCOM produced by two different manufacturers, Manufacturer A (FIG. 9) and Manufacturer B (FIG. 10), was assessed over time using Scanning Electron microscopy. All examined WOUNDCOM specimens exhibit an open porous ultrastructure, with 80% of the pores have a diameter between 20 and 60 μm. The Manufacturer B collagen templates appear more homogenous in ultrastructural appearance regarding pore size distribution. All the evaluated WOUNDCOM patches did not change in their architecture during the tested shelf life.

The effect on SFV33 and GVR28 distribution in WOUNDCOM produced by Collagen two different manufacturers, Manufacturer A and Manufacturer B, was assessed over time (FIG. 11-13). The concentrations of GVR28 and SFV33 in the different WOUNDCOM samples from 5 different WOUNDCOM patches were evaluated by quantitative immune electron microscopy over time (FIGS. 11-13). The number of immunogold particles/μm² were counted at randomly selected areas. The chosen experimental conditions allowed to directly translate the number of immunogold particles/μm² to actual concentrations of target structures (i.e., local GVR28 and SFV33 concentrations).

Both GVR28 and SFV33 were evenly distributed in the collagen scaffold of all examined WOUNDCOM patches. The calculated variations in local concentrations for both peptides were in the range of about +/−10%. No differences were found between WOUNDCOM manufactured by Manufacturer A and Manufacturer B.

GVR28 and SFV33 clearly exhibit an even and reproducible distribution and concentration in all examined WOUNDCOM patches. WOUNDCOM is thus produced by a stable and reproducible manufacturing process.

It can be assumed, since the production technology of WOUNDCOM by Manufacturer B and Manufacturer A is essentially identical, that the long-terms stability of the WOUNDCOM units from Manufacturer B would demonstrate the same stability after 12 months, as the WOUNDCOM units produced by Manufacturer A.

Conclusions

At the different time points assessed no alterations were observed in the fine structure or peptide concentrations of WOUNDCOM, neither in scanning electron microscopic appearance, nor at transmission electron microscopic level.

Taken together, these results indicate that the WOUNDCOM wound dressing has a robust and reproducible production profile and thus a reliable benefit for the management of chronic infected wounds. Furthermore, this evaluation also shows equivalent production of WOUNDCOM from both Manufacturer A and Manufacturer B.

The data shows that WOUNDCOM has a shelf life of at least a year, which is sufficient for effective utilisation of the product in relevant settings (e.g. provides sufficient time for manufacture, distribution and use in a clinic or hospital).

### Example 5—Comparing the Wound Healing and Antimicrobial Effects of WOUNDCOM that Comprises Bioactive Collagen VI Peptides with Other Membranes

The wound healing effects and antimicrobial activity of WOUNDCOM was compared to that of ProHeal. ProHeal is a 100% collagen product and has the same base material as WOUNDCOM and therefore acts a comparator for assessing the effects of the bioactive collagen VI peptides. The Whatman cellulose was used a further control to provide an insight on the contribution of the collagen base material in the increased fibroblast proliferation.

WOUNDCOM comprises a collagen-based carrier in combination with the bioactive collagen VI polypeptides GVR28 and SFV33.

i. In Vitro Tests Assessing Wound Healing Effects of the Membranes

Materials and Methods

The unsterile samples of WOUNDCOM were produced by Manufacturer B. The sterile ProHeal samples (REF 83030-001, Batch no. 2001099) were produced by MedSkin Solutions. Whatman cellulose filter paper (REF 10 311 611, Batch No. G1504017) was used.

Dermal fibroblasts (Lonza #CC2511), FBM-medium (Lonza #CC3131), reagents, utensils etc. were as accounted for below:

| Reagent | Catalogue # | Lot/Batch/Notes | Source |
|---|---|---|---|
| FBM-medium | CC3131 | | Lonza/BioNordika |
| FGM-2 single Quot kit | CC4126 | | Lonza/BioNordika |
| Trypsin - fibroblast | CC5012 | | Lonza/BioNordika |
| T75 flasks | 156499 | | ThermoFisher |
| 24-well plate | 83.3922.500 | | Sarstedt |
| 12-well plate | 174931 | | ThermoFisher |
| PBS (no Ca or Mg) | 14190250 | | Gibco, |
| Resazurin | R7017 | MKB4293V | Sigma-Aldrich |
| Microtiterplates | 655087 | | Greiner |
| 96-wells (black) | | | |
| Biobsy puncher | 173693 | 20408104 | Kruuse |

Protocol

Preparation of Punches
Circular punches from the 6 different wound pads defined in 6.2 are prepared by a tissue 8 mm biopsy puncher under sterile conditions

Preculturing and Seeding of Dermal Fibroblasts
Thaw cells according to Lonza's recommendations specific to dermal fibroblasts (no spinning)
Grow cells in FBM medium with supplements. Use increasing volume of medium as cells become more confluent (1 ml/5 cm² under 25% confluence; 25-45% 1.5 ml/5 cm²; >45% 2 ml/5 cm²)
Cells should be around 60-80% confluent on the day of seeding
Remove medium and wash cells with PBS
Add Lonza Trypsin
After cells are released add the double volume of medium with FBS
Spin for 5 min at 300×g
Aspirate supernatant and resuspend cells in medium
Determine cell concentration and dilute cells.
Punches are placed in 12-well plates with 50 ul medium in the bottom to hold them in place.
Seed cells on the punches
   a. 10.000 cells/50 ul medium (EM) in duplicates or
   b. 20.000 cells/50 ul medium (proliferation) in triplicates
Let cells adhere to the punches for 1 h
Add 1400 ul medium gently to wells
Incubate cells for 0, 24, 48 and 96 hours
For the 96 h samples medium is replaced after 48 h.

Preparation of Cells for Electron Microscopy
Cells incubated for 0, 24, 48 and 96 hours on the pad punches are washed once in PBS
Samples are then fixed in EM fix (supplied by sponsor) and are delivered to the sponsor

The same amount of cells (10.000) were applied to the three sample types (WOUNDCOM, ProHeal and Whatman cellulose filter paper). The wound healing effect of the three different samples were tested in vitro by assessing cell adherence and proliferation on a given sample. This was assessed using the method outlined above).

Results

The results of the in vitro study are presented in FIG. 14. The maximum number of cells counted (119 cells for WOUNDCOM 96 h) was set to 100% on the y-axis.

The same amount of cells were applied to the three sample types. The different starting number indicates that already from t=0, it may be easier for cells to adhere to a material where the collagen VI peptides (GVR28 and SFV33) are present. Thereafter, WOUNDCOM accelerates the fibroblast cell proliferation. The increase in fibroblast density observed for WOUNDCOM was at least 2 times that observed for ProHeal and at least 4 times that observed for the Whatman cellulose filter paper.

In summary, WOUNDCOM exhibits superior wound healing effects in comparison to other membranes.

ii. In Vitro Tests Assessing Antimicrobial Activity of the Membranes

Material and Methods

The samples of WOUNDOM, ProHeal and Whatman cellulose fibres used in this study are the same as outlined in the wound healing experiments above.

The antimicrobial activity of the different samples was assessed in vitro by determining bacterial killing on a given membrane surface by the following method.

Bacterial Strains

Pseudomonas aeruainosa

TABLE 1
Information regarding Pseudomonas aeruginosa CCUG 56489

| Parameter | Value | Comments |
|---|---|---|
| Generic name | Pseudomonas aeruginosa | Class II pathogen |
| | CCUG 56489 (Migula 1900 | |
| | AL, ATCC 15692) | |
| Storage conditions | −80° C. | Glycerol stock |

Staphylococcus aureus

TABLE 2
Information regarding Staphylococcus aureus CCUG 10778

| Parameter | Value | Comments |
|---|---|---|
| Generic name | Staphylococcus aureus | Class II pathogen |
| | CCUG 10778 (Rosenbach | |
| | 1884 VP, ATCC 15692) | |
| Storage conditions | −80° C. | Glycerol stock |

Material and Reagent list

| Chemical/Material | Vendor | Product # | Comment |
|---|---|---|---|
| Tryptic Soy Agar plates | Klinisk mikrobiologi | | |
| (TSA) | (Lund) | | |
| Tryptic Soy Broth (TSB) | Klinisk mikrobiologi | | |
| | (Lund) | | |
| Erlenmeyer flasks | | | |
| Gosselin plastic spreader | Fisher | 11836191 | |
| BRAND semi-micro cuvette | Sigma | BR759015 | |
| Inoculation loop | ThermoFisher | 254410 | |
| Skin punch biopsy tool | Kruuse | 273693 | |
| 8 mm diameter | | | |
| 1.5 mL microtube | Sarstedt | | |
| Tweezer | AgnTho's | | |
| EM fixer media | | | From Sponsor |
| KH₂PO₄ Dibasic | Sigma | 795496 | |

Instruments

TABLE 9
Information regarding equipment used

| | | | |
|---|---|---|---|
| Chemical/Material | Vendor | Product # | Comments |

| | | |
|---|---|---|
| Heratherm IGS180 Incubator | ThermoFisher | 51028065 |
| Compact Digital Mini Rotator | ThermoFisher | 15278844 |
| Cell Density Meter | ThermoFisher | 11899283 |
| Megastar 1.6 Centrifuge | VWR | 521-1749 |
| Vortex-Genie 2 | Scientific | |
| | Industries | |

Growing of Bacterial Cultures

The following procedures are identical for S. aureus and P. aeruginosa.

Within a week prior to the experiment, a small loopful of bacterial stock was streaked on non-selective agar plates (TSA) and incubated overnight at 37° C.±1° C. The cultures were examined visually for purity prior to use. All bacterial colonies were examined to check they exhibit the same colony morphology and color.

The day prior to the experiment (day −1), a small loopful from the TSA plates was used to inoculate two sterile 50 mL Erlenmeyer flasks containing 20 mL TSB medium. The flasks were incubated at 37° C.±1° C. with shaking (300 rpm).

The day of the experiment (day 0), an aliquot from the overnight cultures was diluted 10× in TSB medium and OD₆₀₀ was measured. From previous measurements, overnight cultures grow to a density of approximately 10⁹ CFUs/mL.

Preparing Samples for Electron Microscopy Analysis

Test item samples were placed in sterile 24-well plates. An aliquot from the overnight culture was serially diluted and plated on TSA plates to determine the cell density. 50 μL aliquots from the overnight cultures were pipetted onto the test item samples and incubated for 0 h (10 s), 0.5 h, 1 h and 2 h at 37±1° C. in a wet chamber. At the end of each incubation time, the test item samples will be placed in 1 mL EM fix in a sterile 24-well plate and handed over to the sponsor for EM analysis to determine activity/survival.

Results

The results of the in vitro antimicrobial activity experiments are presented in FIG. 15. After 30 minutes of exposure a reduction in bacterial survival on WOUNDCOM was observed. Specifically, an 18% reduction in survival of Staphylococcus aureus was observed and a reduction in survival of 25% of Pseudomonas aeruginosa was observed. After 60 minutes the bacterial survival rate on the surface of WOUNDCOM samples was 30% for S. aureus and 20% for P. auruginosa, representing a reduction in bacterial survival of 70% and 80% respectively. At the end of the study (120 minutes) the survival of S. aureus and P. aeruginosa on the surface of WOUNDCOM samples was below 10%. Over the entire study (0 to 120 minutes), the comparators membranes only showed limited (ProHeal) or no detectable (cellulose filter paper) antimicrobial effect.

The results collected from this study demonstrate that WOUNDCOM has antimicrobial efficacy against bacteria such as Staphylococcus aureus and Pseudomonas aeruginosa and the antimicrobial effects are superior to the other membranes tested (ProHeal and the filter paper).

### REFERENCES

1. Gurtner G C, Werner S, Barrandon Y, Longaker M T. Wound repair and regeneration. Nature 2008; 453:314-21.
2. Sen C K, Gordillo G M, Roy S, Kirsner R, Lambert L, Hunt T K, Gottrup F, Gurtner G C, Longaker M T. Human skin wounds: a major and snowballing threat to public health and the economy. Wound Repair Regen 2009; 17:763-71.
3. Moura L I, Dias A M, Carvalho E, de Sousa H C. Recent advances on the development of wound dressings for diabetic foot ulcer treatment—a review. Acta Biomater 2013; 9:7093-114.
4. Guo S, Dipietro L A. Factors affecting wound healing. J Dent Res 2010; 89:219-29.
5. Sen C K, Gordillo G M, Roy S, Kirsner R, Lambert L, Hunt T K, Gottrup F, Gurtner G C, Longaker M T. Human skin wounds: a major and snowballing threat to public health and the economy. Wound Repair Regen 2009; 17:763-71.
6. Shah J M Y, Omar E, Pai D R, Sood S. Cellular events and biomarkers of wound healing. Indian J Plast Surg 2012; 45:220-28.
7. Weller C, Sussman G. Wound dressings update. 3 Pharm Prac Res 2006; 36:318-24.
8. Pereira R F, Barrias C C, Granja P L, Bartolo P3. Advanced biofabrication strategies for skin regeneration and repair. Nanomedicine (Lond) 2013; 8:603-21.
9. Martin, P. Wound healing—aiming for perfect skin regeneration. Science 1997; 276:75-81.
10. Kirby, G. T., Mills, S. J., Cowin, A. J., Smith, L. E. Stem cells for cutaneous wound healing. Biomed Res Int 2015; 2015:285869.
11. Isakson M, de Blacam C, Whelan D, McArdle A, Clover A J Mesenchymal stem cells and cutaneous wound healing: current evidence and future potential. Stem Cells Int 2015; 2015:831095.
12. Sinno H, Prakash S. Complements and the wound healing cascade: an updated review. Plast Surg Int 2013; 2013:146764.
13. Wound healing: an overview. Broughton G 2nd, Janis J E, Attinger C E. Plast Reconstr Surg 2006; 117(7 Suppl): 1e-S-32e-S.
14. Das S, Baker A B. Biomaterials and Nanotherapeutics for Enhancing Skin Wound Healing. Front Bioeng Biotechno 2016; 4:82. eCollection 2016.
15. Hunt T K. The physiology of wound healing. Ann Emerg Med 1988; 17:1265-73
16. Abou Neel E A, Bozec L, Knowles J C, Syed O, Mudera V, Day R, Hyun J K. Collagen—Emerging collagen based therapies hit the patient Adv Drug Deliv Rev 2013; 65:429-56.
17. An B, Lin Y S, Brodsky B. Collagen interactions: Drug design and delivery. Adv Drug Deliv Rev 2016; 97:69-84.
18. Collagen-based biomaterials for tissue engineering applications. Parenteau-Bareil R., Gauvin R., Berthod F. Materials 2010; 3:1863-87.
19. Chattopadhyay S, Raines R T. Review collagen-based biomaterials for wound healing. Biopolymers 2014; 101:821-33.
20. Baum C L, Arpey C J. Normal cutaneous wound healing: clinical correlation with cellular and molecular events. Dermatol Surg 2005; 31:674-86.
21. Ghica M V, Albu Kaya M G, Dinu-Pirvu C E, Lupuleasa D, Udeanu D I. Development, Optimization and In Vitro/In Vivo Characterization of Collagen-Dextran Spongious Wound Dressings Loaded with Flufenamic Acid. Molecules 2017; 22:1552.
22. Guo R, Lan Y, Xue W, Cheng B, Zhang Y, Wang C, Ramakrishna S. Collagen-cellulose nanocrystal scaffolds containing curcumin-loaded microspheres on infected full-thickness burns repair. J Tissue Eng Regen Med 201741:3544-55.
23. Bhowmick S., Thanusha A. V., Kumar A., Scharnweber D., Rother S., Koul V. (2018). Nanofibrous artificial skin substitute composed of mPEG-PCL grafted gelatin/hyaluronan/chondroitin sulfate/sericin for 2nd degree burn care: in vitro and in vivo study. RSC Adv 2018; 8:16420-32.
24. Yoon D, Yoon D, Cha H J, Lee J S, Chun W. Enhancement of wound healing efficiency mediated by artificial dermis functionalized with EGF or NRG1. Biomed Mater 2018; 13: 045007.
25. Powell H M, Supp D M, Boyce S T. Influence of electrospun collagen on wound contraction of engineered skin substitutes. Biomaterials 2008; 29:834-43.
26. Hall Barrientos 13, Paladino E, Szabó P, Brozio S, Hall P J, Oseghale C I, Passarelli M K, Moug S J, Black R A, Wilson C G, Zelkó R, Lamprou D A. Electrospun collagen-based nanofibres: A sustainable material for improved antibiotic utilisation in tissue engineering applications. Int J Pharm 2017; 531:67-79.
27. Golser A. V., Ro M., Bo H. G., Scheibel T. Engineered collagen: a redox switchable framework for tunable assembly and fabrication of biocompatible surfaces. ACS Biomater Sci Eng 2018; 4:2106-14.
28. Velnar T, Bailey T, Smrkolj V. The wound healing process: an overview of the cellular and molecular mechanisms. J Int Med Res 2009; 37:1528-42.
29. Abdillahi S M, Maaß T, Kasetty G, Strömstedt A A, Baumgarten M, Tati R, Nordin S L, Walse B, Wagener R, Schmidtchen A, Morgelin M. Collagen VI Contains Multiple Host Defense Peptides with Potent In Vivo Activity J Immunol 2018; 201:1007-1020.
30. Yount, N. Y., Bayer, A. S., Xiong, Y. Q., and Yeaman, M. R. (2006) Advances in antimicrobial peptide Immunobiology. Biopolymers 84, 435-458
31. Teixeira, V., Feio, M. J., and Bastos, M. (2012) Role of lipids in the interaction of antimicrobial peptides with membranes. Progress in lipid research 51, 149-177
32. Specks, U., Mayer, U., Nischt, R., Spissinger, T., Mann, K., Timpl, R., Engel, J., and Chu, M. L. (1992) Structure of recombinant N-terminal globule of type VI collagen alpha 3 chain and its binding to heparin and hyaluronan. The EMBO journal 11, 4281-4290
33. Chu, M. L., Pan, T. C., Conway, D., Kuo, H. J., Glanville, R. W., Timpl, R., Mann, K., and Deutzmann, R. (1989) Sequence analysis of alpha 1(VI) and alpha 2(VI) chains of human type VI collagen reveals internal triplication of globular domains similar to the A domains of von Willebrand factor and two alpha 2(VI) chain variants that differ in the carboxy terminus. The EMBO journal 8, 1939-1946
34. Chu, M. L., Zhang, R. Z., Pan, T. C., Stokes, D., Conway, D., Kuo, H. J., Glanville, R., Mayer, U., Mann, K., Deutzmann, R., and et al. (1990) Mosaic structure of globular domains in the human type VI collagen alpha 3 chain: similarity to von Willebrand factor, fibronectin, actin, salivary proteins and aprotinin type protease inhibitors. The EMBO journal 9, 385-393
35. Chu, M. L., Pan, T. C., Conway, D., Saitta, B., Stokes, D., Kuo, H. J., Glanville, R. W., Timpl, R., Mann, K., and Deutzmann, R. (1990) The structure of type VI collagen. Annals of the New York Academy of Sciences 580, 55-63
36. Lamande, S. R., Mörgelin, M., Adams, N. E., Selan, C., and Allen, J. M. (2006) The C5 domain of the collagen type VI alpha3(VI) chain is critical for extracellular microfibril formation and is present in the extracellular matrix of cultured cells. The Journal of biological chemistry 281, 16607-16614
37. Cescon, M., Gattazzo, F., Chen, P., and Bonaldo, P. (2015) Collagen VI at a glance. Journal of cell science 128, 3525-3531
38. Isenhath, S. N., Fukano, Y., Usui, M. L., Underwood, R. A., Irvin, C. A., Marshall, A. J., Hauch, K. D., Ratner, B. D., Fleckman, P., and Olerud, J. E. (2007) A mouse model to evaluate the interface between skin and a percutaneous device. J Biomedical and Material Research 83A, 915-922
39. Lin Q., Fang, D., Fang, J., Ren, X., Yang, X., Wen, F., and Su, S. B. (2011) Impaired wound healing with defective expression of chemokines and recruitment of myeloid cells in TLP3-deficient mice. Journal of Immunology 186, 3710-3717
40. Canesso, M. C. C., Vieira, A. T., Castro, T. B. R., Schirmer, B. G. A., Cisalpino, D., Martins, F. S., Rachid, M. A., Nicoli, J. R., Teixeira, M. M., and Barcelos, L. S. (2014) Skin Wound Healing Is Accelerated and Scarless in the Absence of Commensal Microbiota. Journal of Immunology 193, 5171-5180
41. Terhorst, D., Chelbi, R., Wohn, C., Malosse, C., Tamoutounour, S., Jorquera, A., Bajenoff, M., Dalod, M., Malissen, B., and Henri, S. (2015) Dynamics and Transcriptomics of Skin Dendritic Cells and Macrophages in an Imiquimod-Induced, Biphasic Mouse Model of Psoriasis. Journal of Immunology 195, 4953-4961
42. Punjataewakupt, A., Napavichayanun, S., Aramwit, P. (2019) The downside of antimicrobial agents for wound healing. Eur J Clin Microbial Infect Dis., 38(1):39-54
43. Dill V, Morgelin M (2020) Biological Dermal Templates with Native Collagen Scaffolds Provide Guiding Ridges for Invading Cells and may Promote Structured Dermal Wound Healing. Int Wound J 17:618-30.
44. Tati R, Nordin 5, Abdillahi S M, Mörgelin M. (2018) Biological Wound Matrices with Native Dermis-like Collagen Efficiently Modulate Protease Activity. J Wound Care 27:199-209.
45. Tajima S, Ting J P Y, Pinnell S R, Kaufman R E. (1984) Isolation and Characterization of a Human Pro alpha 2(I) Collagen Gene Segment. J Invest Derm 82:265-69.
46. Ricard-Blum S. (2011) The Collagen Family. Cold Spring Harb Perspect Biol 3:a004978.
47. Buckley M (2016) Species Identification of Bovine, Ovine and Porcine Type 1 Collagen; Comparing Peptide Mass Fingerprinting and L C-based Proteomics Methods. Int J Mol Sci 17:445.
48. Junker J P E, Kamel R A, Caterson E J, Eriksson O. (2013) Clinical Impact upon Wound Healing and Inflammation in Moist, Wet and Dry Environments. Adv Wound Care (New Rochelle) 2:348-56.
49. Abdillahi S M, Maaβ T, Kasetty G, Strömstedt A A, Baumgarten M, Tati R, Nordin S L, Walse B, Wagener R, Schmidtchen A, Mörgelin M. (2018) Collagen VI Contains Multiple Host Defense Peptides with Potent In Vivo Activity. J Immunol 201:1007-1020.
50. Jianbo Sun, Yuqiong Xia, Dong Li, Quan Du, Dehai Liang. Relationship between peptide structure and antimicrobial activity as studied by de novo designed peptides. Biochimica et Biophysica Acta (BBA)—Biomembranes Volume 1838, Issue 12, December 2014, Pages 2985-2993. Doi:10.1016/j.bbamem.2014.08.018
51. Hao-Ting Lee, Chen-Che Lee, Je-Ruei Yang, Jim Z. C. Lai, Kuan Y. Chang. A Large-Scale Structural Classification of Antimicrobial Peptides. BioMed Research International 2015:1-6.Doi: 10.1155/2015/475062

1. A composition comprising:
(a) a first collagen type VI polypeptide comprising or consisting of an amino acid sequence derived from collagen type VI, or a fragment, variant, fusion or derivative thereof, or a fusion of said fragment, variant of derivative thereof, wherein the first polypeptide has the primary activity of being capable of promoting wound healing; and
(b) a second collagen type VI polypeptide comprising or consisting of an amino acid sequence derived from collagen type VI, or a fragment, variant, fusion or derivative thereof, or a fusion of said fragment, variant of derivative thereof, wherein the second polypeptide has the primary activity of being capable of exerting an antimicrobial effect.

2. (canceled)

3. (canceled)

4. The composition of claim 1, wherein the first and/or second polypeptide fragment, variant, fusion or derivative:
a) is capable of binding to the membrane of the microorganism; and/or
b) is capable of causing membrane disruption of the microorganisms; and/or
c) is capable of enhancing epithelia (comprising epidermal) regeneration; and/or
d) is capable of enhancing healing of wound epithelia (comprising epidermis); and/or
e) is capable of enhancing healing of wound stroma (comprising dermis); and/or
f) is capable of exhibiting an antimicrobial effect greater than or equal to that of LL-37; and/or
g) is substantially non-toxic to mammalian cells; and/or
h) is capable of exerting an anti-endotoxic effect; and/or
i) is derived from a von Willebrand Factor type A domain; and/or
j) is or is derived from the α1, α2 and/or α3 chain of collagen type VI, preferably wherein the first and/or second polypeptide is or is derived from the α3 chain of collagen type VI; and/or
k) is or is derived from the N2, N3 or Cl domain of the α3 chain of collagen type VI; and/or
l.) has a net positive charge, optionally wherein the charge on the first and/or second polypeptide ranges from between +2 to +9, preferably wherein the charges are different or the same; and/or
m) has at least 30% hydrophobic residues; and/or
n) is a recombinant polypeptide; and/or
o) is capable of killing or attenuating the growth of microorganisms, optionally wherein the microorganisms are selected from the group consisting of bacteria, mycoplasmas, yeasts, fungi and viruses; and/or
p) is capable of promoting wound closure; and/or
q) comprises one or more amino acids that are modified or derivatised, optionally wherein the one or more amino acids are modified or derivatised by PEGylation, amidation, esterification, acylation, acetylation and/or alkylation.

5-8. (canceled)

9. The composition according to claim 1, wherein the antimicrobial effect is against microorganisms which are Gram-positive or Gram-negative bacteria, optionally wherein the microorganisms are selected from the group consisting of: Pseudomonas aeruginosa, Staphylococcus aureus, Escherichia coli, group A streptococcus (e.g. Streptococcus pyogenes), group B streptococcus (e.g. Streptococcus agalactiae), group C streptococcus (e.g. Streptococcus dysgalactiae), group D streptococcus (e.g. Enterococcus faecalis), group F streptococcus (e.g. Streptococcus anginosus), group G streptococcus (e.g. Streptococcus dysgalactiae equisimilis), alpha-hemolytic streptococcus (e.g. Streptococcus viridans, Streptococcus pneumoniae), Streptococcus bovis, Streptococcus mitis, Streptococcus anginosus, Streptococcus sanguinis, Streptococcus suis, Streptococcus mutans, Moraxella catarrhalis, Non-typeable Haemophilus influenzae (NTHi), Haemophilus influenzae b (Hib), Actinomyces naeslundii, Fusobacterium nucleatum, Prevotella intermedia, Klebsiella pneumoniae, Enterococcus cloacae, Enterococcus faecalis, Staphylococcus epidermidis, multidrug-resistant Pseudomonas aeruginosa (MRPA), multidrug-resistant Staphylococcus aureus (MRSA), multidrug-resistant Escherichia coli (MREC), multidrug-resistant Staphylococcus epidermidis (MRSE), multidrug-resistant Klebsiella pneumoniae (MRKP), multidrug-resistant Enterococcus faecium (MREF), multidrug-resistant Acinetobacter baumannii (MRAB) and multidrug-resistant Enterobacter spp. (MRE), optionally wherein the microorganisms are bacteria which are resistant to one or more conventional antibiotic agents, optionally wherein the microorganism is selected from the group consisting of: multidrug-resistant Staphylococcus aureus (MRSA), multidrug-resistant Pseudomonas aeruginosa (MRPA), multidrug-resistant Escherichia coli (MREC), multidrug-resistant Staphylococcus epidermidis (MRSE) and multidrug-resistant Klebsiella pneumoniae (MRKP).

10-21. (canceled)

22. The composition of claim 1, wherein the first and/or second polypeptide comprises or consists of the amino acid sequence selected from the group consisting of SEQ ID NOs: 1 to 23, and fragments, variants, fusions or derivatives thereof, and fusions of said fragments, variants and derivatives thereof, which retain an antimicrobial activity of any one of SEQ ID NOs:1 to 23.

23. The composition of claim 1, wherein the first and/or second polypeptide comprises or consists of the amino acid sequence selected from the group consisting of SEQ ID NOs: 1 to 5:
″GVR28″:
[SEQ ID NO: 1]
GVRPDGFAHIRDFVSRIVRRLNIGPSKV
″FYL25″:
[SEQ ID NO: 2]
FYLKTYRSQAPVLDAIRRLRLRGGS
″FFL25″:
[SEQ ID NO: 3]
FFLKDFSTKRQIIDAINKVVYKGGR
″VTT30″:
[SEQ ID NO: 4]
VTTEIRFADSKRKSVLLDKIKNLQVALTSK
″SFV33″:
[SEQ ID NO: 5]
SFVARNTFKRVRNGFLMRKVAVFFSNTPTRASP
and fragments, variants, fusions or derivatives thereof, and fusions of said fragments, variants and derivatives thereof, which retain an antimicrobial activity of any one of SEQ ID NOs:1 to 5.

24. (canceled)

25. (canceled)

26. The composition of claim 1, wherein the variant of the first and/or second polypeptide has at least 50% identity with the amino acid sequence amino acid sequence of any one of SEQ ID NOs: 1 to 23, for example at least 55%, 60%, 65%, 70%, 75%, 80%, 85%, 90%, 95%, 96%, 97%, 98% or at least 99% identity.

27. The composition of claim 1, wherein the first and/or second polypeptide is:
(a) is between 10 and 200 amino acids in length, for example between 10 and 150, 15 and 100, 15 and 50, 20 and 40, 25 and 35, or 28 and 33 amino acids in length; and/or
(b) is part of a longer amino acid sequence, wherein the first and/or second polypeptide is part of an amino acid sequence that is up to 25, 28, 30, 33, 35, 40, 45, 50, 55, 60, 65, 70, 75, 80, 85, 90, 95 or 100 amino acids in length; and/or
(c) is part of a longer amino acid sequence, wherein the first and/or second polypeptide is part of an amino acid sequence that is between 20 and 200, 28 and 200, 33 and 200, 28 and 150, 33 and 150, 28 and 100, 33 and 100, 28 and 50, 33 and 50, 28 and 40, 33 and 40, or 28 and 33 amino acids in length; and/or
(d) is at least 20 amino acids in length, preferably at least 28 amino acids in length; and/or
(e) are present in the composition at a ratio of at least 0.5:1; for example, 0.6:1, 0.7:1, 0.8:1, 0.9:1 or 1:1; or are present in the composition at a ratio of at least 0.5:1; for example, 0.6:1, 0.7:1, 0.8:1, 0.9:1 or 1:1.

28-32. (canceled)

33. The composition of claim 1, wherein:
(a) the first polypeptide comprises or consists of the amino acid sequence according to SEQ ID NO:1 (i.e. GVR28), or fragments, variants, fusions or derivatives thereof and fusions of said fragments, variants and derivatives thereof which retain the wound healing activity of SEQ ID NO: 1; and/or
(b) the second polypeptide comprises or consists of the amino acid sequence according to SEQ ID NO:5 (i.e. SFV33), or fragments, variants, fusions or derivatives thereof, and fusions of said fragments, variants and derivatives thereof, which retain an antimicrobial activity of SEQ ID NO: 5.

34. (canceled)

35. The composition of claim 1, wherein the composition further comprises a scaffold material, optionally wherein the scaffold is collagen I.

36. (canceled)

37. A pharmaccutical composition of claim 1 further comprising a pharmaceutically acceptable excipient, diluent, carrier, buffer or adjuvant.

38. A medical device, implant, wound care product, or material for use in the same, which is coated, impregnated, admixed or otherwise associated with a composition of claim 1.

39. (canceled)

40. The medical device, implant, wound care product, or material for use in the same, according to claim 38, wherein
(a) the device, implant, wound care product, or material is for use in by-pass surgery, extracorporeal circulation, wound care and/or dialysis; and/or
(b) the composition is coated, painted, sprayed or otherwise applied to a suture, prosthesis, implant, wound dressing, catheter, lens, skin graft, skin substitute, fibrin glue or bandage; and/or
(c) wherein the medical device, implant, wound care product, or material for use in the same further comprises a polymer, metal, metal oxide and/or ceramic.

41-59. (canceled)

60. A method of treating an individual with a microbial infection or a wound, the method comprising the step of administering to an individual in need thereof an effective amount of a composition according to claim 1.

61-67. (canceled)

68. The composition of claim 1 wherein:
(a) the first polypeptide comprises or consists of the amino acid sequence according to SEQ ID NO:1 (i.e. GVR28), or fragments, variants, fusions or derivatives thereof and fusions of said fragments, variants and derivatives thereof which retain the wound healing activity of SEQ ID NO: 1; and
(b) the second polypeptide comprises or consists of the amino acid sequence according to SEQ ID NO:5 (i.e. SFV33), or fragments, variants, fusions or derivatives thereof, and fusions of said fragments, variants and derivatives thereof, which retain an antimicrobial activity of SEQ ID NO: 5.

69. The medical device, implant, wound care product, or material for use in the same, according to claim 38 comprising:
(a) a scaffold material, wherein the scaffold is collagen I.
(b) a first polypeptide comprising or consisting of the sequence of GVR28:
″GVR28″:
[SEQ ID NO: 1]
GVRPDGFAHIRDFVSRIVRRLNIGPSKV;
and
(c) a second polypeptide comprising or consisting of the sequence of SFV33:
″SFV33″:
[SEQ ID NO: 5]
SFVARNTFKRVRNGFLMRKVAVFFSNTPTRASP.

70. The method of treating an individual of claim 60 wherein the first and/or second polypeptide of the composition comprises or consists of the amino acid sequence selected from the group consisting of SEQ ID NOs: 1 to 23, and fragments, variants, fusions or derivatives thereof, and fusions of said fragments, variants and derivatives thereof, which retain an antimicrobial activity of any one of SEQ ID NOs: 1 to 23.

71. The method of treating an individual of claim 60 wherein the first and/or second polypeptide of the composition comprises or consists of the amino acid sequence selected from the group consisting of SEQ ID NOs: 1 to 5:
″GVR28″:
[SEQ ID NO: 1]
GVRPDGFAHIRDFVSRIVRRLNIGPSKV
″FYL25″:
[SEQ ID NO: 2]
FYLKTYRSQAPVLDAIRRLRLRGGS
″FFL25″:
[SEQ ID NO: 3]
FFLKDFSTKRQIIDAINKVVYKGGR
″VTT30″:
[SEQ ID NO: 4]
VTTEIRFADSKRKSVLLDKIKNLQVALTSK
″SFV33″:
[SEQ ID NO: 5]
SFVARNTFKRVRNGFLMRKVAVFFSNTPTRASP
and fragments, variants, fusions or derivatives thereof, and fusions of said fragments, variants and derivatives thereof, which retain an antimicrobial activity of any one of SEQ ID NOs:1 to 5.

72. The method of treating an individual of claim 60 wherein the first and/or second polypeptide of the composition:
(i) is between 10 and 200 amino acids in length, for example between 10 and 150, 15 and 100, 15 and 50, 20 and 40, 25 and 35, or 28 and 33 amino acids in length; and/or
(ii) is part of a longer amino acid sequence, wherein the first and/or second polypeptide is part of an amino acid sequence that is up to 25, 28, 30, 33, 35, 40, 45, 50, 55, 60, 65, 70, 75, 80, 85, 90, 95 or 100 amino acids in length; and/or
(iii) is part of a longer amino acid sequence, wherein the first and/or second polypeptide is part of an amino acid sequence that is between 20 and 200, 28 and 200, 33 and 200, 28 and 150, 33 and 150, 28 and 100, 33 and 100, 28 and 50, 33 and 50, 28 and 40, 33 and 40, or 28 and 33 amino acids in length; and/or
(iv) is at least 20 amino acids in length, preferably at least 28 amino acids in length; and/or
(v) are present in the composition at a ratio of at least 0.5:1; for example, 0.6:1, 0.7:1, 0.8:1, 0.9:1 or 1:1; or are present in the composition at a ratio of at least 0.5:1; for example, 0.6:1, 0.7:1, 0.8:1, 0.9:1 or 1:1; and/or
(vi) has at least 50% identity with the amino acid sequence amino acid sequence of any one of SEQ ID NOs: 1 or SEQ ID NO: 5, for example at least 55%, 60%, 65%, 70%, 75%, 80%, 85%, 90%, 95%, 96%, 97%, 98% or at least 99% identity.

73. The method of treating an individual of claim 60 wherein:
(a) the first polypeptide of the composition comprises or consists of the amino acid sequence according to SEQ ID NO:1 (i.e. GVR28), or fragments, variants, fusions or derivatives thereof and fusions of said fragments, variants and derivatives thereof which retain the wound healing activity of SEQ ID NO: 1; and/or
(b) the second polypeptide of the composition comprises or consists of the amino acid sequence according to SEQ ID NO:5 (i.e. SFV33), or fragments, variants, fusions or derivatives thereof, and fusions of said fragments, variants and derivatives thereof, which retain an antimicrobial activity of SEQ ID NO: 5.

74. The method of treating an individual of claim 60 wherein:
(a) the first polypeptide of the composition comprises or consists of the amino acid sequence according to SEQ ID NO:1 (i.e. GVR28), or fragments, variants, fusions or derivatives thereof and fusions of said fragments, variants and derivatives thereof which retain the wound healing activity of SEQ ID NO: 1; and
(b) the second polypeptide of the composition comprises or consists of the amino acid sequence according to SEQ ID NO:5 (i.e. SFV33), or fragments, variants, fusions or derivatives thereof, and fusions of said fragments, variants and derivatives thereof, which retain an antimicrobial activity of SEQ ID NO: 5.


SEQUENCE LISTING (24 sequences)

SEQ ID NO: 1 - protein, 28 aa, Artificial Sequence
  GVR28
GVRPDGFAHIRDFVSRIVRRLNIGPSKV

SEQ ID NO: 2 - protein, 25 aa, Artificial Sequence
  FYL25
FYLKTYRSQAPVLDAIRRLRLRGGS

SEQ ID NO: 3 - protein, 25 aa, Artificial Sequence
  FFL25
FFLKDFSTKRQIIDAINKVVYKGGR

SEQ ID NO: 4 - protein, 30 aa, Artificial Sequence
  VTT30
VTTEIRFADSKRKSVLLDKIKNLQVALTSK

SEQ ID NO: 5 - protein, 33 aa, Artificial Sequence
  SFV33
SFVARNTFKRVRNGFLMRKVAVFFSNTPTRASP

SEQ ID NO: 6 - protein, 32 aa, Artificial Sequence
  DVN32
DVNVFAIGVEDADEGALKEIASEPLNMHMFNL

SEQ ID NO: 7 - protein, 20 aa, Artificial Sequence
  KPE20
KPEILNLVKRMKIKTGKALN

SEQ ID NO: 8 - protein, 20 aa, Artificial Sequence
  GFA20
GFAHIRDFVSRIVRRLNIGP

SEQ ID NO: 9 - protein, 20 aa, Artificial Sequence
  QAP20
QAPVLDAIRRLRLRGGSPLN

SEQ ID NO: 10 - protein, 20 aa, Artificial Sequence
  KGF20
KGFESKVDAILNRISQMHRV

SEQ ID NO: 11 - protein, 20 aa, Artificial Sequence
  RKV20
RKVAVFFSNTPTRASPQLRE

SEQ ID NO: 12 - protein, 20 aa, Artificial Sequence
  VAA20
VAAKPVATKMAVRPPVAVKP

SEQ ID NO: 13 - protein, 20 aa, Artificial Sequence
  AAK20
AAKPVATKPEVPRPQAAKPA

SEQ ID NO: 14 - protein, 20 aa, Artificial Sequence
  TTK20
TTKPVTTTKPVTTTTKPVTT

SEQ ID NO: 15 - protein, 76 aa, Artificial Sequence
  AAA76
AAAKPAPAKPVAAKPVATKMATVRPPVAVKPATAAKPVAAKPAAVRPPAAAAAKPVATKPEVPRPQAAKPAATKPA

SEQ ID NO: 16 - protein, 36 aa, Artificial Sequence
  TSS36
TSSPTSNPVTTTKPVTTTKPVTTTTKPVTTTTKPVT

SEQ ID NO: 17 - protein, 20 aa, Artificial Sequence
  YDR20
YDRLIKESRRQKTRVFAVVI

SEQ ID NO: 18 - protein, 20 aa, Artificial Sequence
  EQN20
EQNFHKARRFVEQVARRLTL

SEQ ID NO: 19 - protein, 20 aa, Artificial Sequence
  VVH20
VVHAINAIVRSPRGGARRHA

SEQ ID NO: 20 - protein, 20 aa, Artificial Sequence
  LRL20
LRLKPYGALVDKVKSFTKRF

SEQ ID NO: 21 - protein, 20 aa, Artificial Sequence
  FTK20
FTKRFIDNLRDRYYRCDRNL

SEQ ID NO: 22 - protein, 20 aa, Artificial Sequence
  RDA20
RDALKSSVDAVKYFGKGTYT

SEQ ID NO: 23 - protein, 20 aa, Artificial Sequence
  TKR20
TKRFAKRLAERFLTAGRTDP

SEQ ID NO: 24 - protein, 37 aa, Artificial Sequence
  Polypeptide
LLGDFFRKSKEKIGKEFKRIVQRIKDFLRNLVPRTES